SEQUENCE LISTING (53 sequences)

SEQ ID NO: 1 - protein, 13 aa, synthetic construct
VASGFTFSHSSMN

SEQ ID NO: 2 - protein, 10 aa, synthetic construct
YISRATGAVY

SEQ ID NO: 3 - protein, 8 aa, synthetic construct
AREPVFDY

SEQ ID NO: 4 - protein, 11 aa, synthetic construct
RASQDISNYLA

SEQ ID NO: 5 - protein, 8 aa, synthetic construct
YAASSLQS

SEQ ID NO: 6 - protein, 9 aa, synthetic construct
LQWSSYPRT

SEQ ID NO: 7 - protein, 115 aa, synthetic construct
QVQLVESGGGLVQPGGSLRLSCVASGFTFSHSSMNWVRQAPGKGLEWVSYISRATGAVYYADSVKGRFTISRDNAKNSLYLQMNSLRDEDTAVYYCAREPVFDYWGQGTLVTVSS

SEQ ID NO: 8 - protein, 107 aa, synthetic construct
DIQMTQSPSAMSASVGDRVTITCRASQDISNYLAWFQQKPGKVPTRLIYAASSLQSGVPSRFSGSGSGTEFTLTISSLQPEDFATYYCLQWSSYPRTFGQGTKVEIK

SEQ ID NO: 9 - protein, 441 aa, synthetic construct
QVQLVESGGGLVQPGGSLRLSCVASGFTFSHSSMNWVRQAPGKGLEWVSYISRATGAVYYADSVKGRFTISRDNAKNSLYLQMNSLRDEDTAVYYCAREPVFDYWGQGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDICVEWESNGQPENNYKTTPPVLDSDGSFFLYSRLTVDKSRWQQGNVFSCSVMHEALHNHYTQKSLSLSLG

SEQ ID NO: 10 - protein, 214 aa, synthetic construct
DIQMTQSPSAMSASVGDRVTITCRASQDISNYLAWFQQKPGKVPTRLIYAASSLQSGVPSRFSGSGSGTEFTLTISSLQPEDFATYYCLQWSSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1323 bp, synthetic construct
  source 1..1323: mol_type = other DNA
caggtacagctggtggagtctgggggaggcttggtacagcctggggggtccctgagactctcctgtgtcgcctctggattcaccttcagtcattctagcatgaactgggtccgccaggctccagggaaggggctggagtgggtttcatacattagtcgtgctactggtgccgtctactacgcagactctgtaaagggccgattcaccatctccagagataatgccaaaaactcactgtatctgcaaatgaacagcctgagagacgaggacacggctgtgtattactgtgcgagagagccggtttttgactactggggccagggaaccctggtcaccgtctcctcagcctccaccaagggcccatgcgtcttcccgctagcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtcaagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccgagaggagatgaccaagaaccaggtcagcctgacctgcctggtcaaaggcttctaccccagcgacatctgcgtggagtgggaaagcaatgggcagccggagaacaactacaagaccacgcctcccgtgctggactccgacggctccttcttcctctacagcaggctaaccgtggacaagagcaggtggcagcaggggaatgtcttctcatgctccgtgatgcatgaggctctgcacaaccactacacacagaagagcctctccctgtctctgggt

SEQ ID NO: 12 - DNA, 642 bp, synthetic construct
  source 1..642: mol_type = other DNA
gacatccagatgacccagtctccatctgccatgtctgcatctgtgggagacagagtcaccatcacttgtcgggcgagtcaggacattagcaattatttagcctggtttcagcagaaaccagggaaagtccctacgcgcctgatctatgctgcatccagtttgcaaagtggggtcccatcaaggttcagcggcagtggatctgggacagaattcactctcacaatcagcagcctgcagcctgaagattttgcaacttattactgtctacagtggtccagttaccctcggacgttcggccaagggaccaaggtggaaatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 13 - protein, 445 aa, synthetic construct
QVQLVESGGGLVQPGGSLRLSCVASGFTFSHSSMNWVRQAPGKGLEWVSYISRATGAVYYADSVKGRFTISRDNAKNSLYLQMNSLRDEDTAVYYCAREPVFDYWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVKGFYPSDICVEWESNGQPENNYKTTPPVLDSDGSFFLYSKLTVDKSRWQQGNVFSCSVMHEALHNHYTQKSLSLSPGK

SEQ ID NO: 14 - DNA, 1335 bp, synthetic construct
  source 1..1335: mol_type = other DNA
caggtacagctggtggagtctgggggaggcttggtacagcctggggggtccctgagactctcctgtgtcgcctctggattcaccttcagtcattctagcatgaactgggtccgccaggctccagggaaggggctggagtgggtttcatacattagtcgtgctactggtgccgtctactacgcagactctgtaaagggccgattcaccatctccagagataatgccaaaaactcactgtatctgcaaatgaacagcctgagagacgaggacacggctgtgtattactgtgcgagagagccggtttttgactactggggccagggaaccctggtcaccgtctcctcagctagcaccaagggcccatgcgtcttccccctggcaccctcctccaagagcacctctgggggcacagcggccctgggctgcctggtcaaggactacttccccgaaccggtgacggtgtcgtggaactcaggcgcactgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcgccgtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatctgcgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 15 - protein, 207 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 16 - protein, 207 aa, Macaca fascicularis
MKVLQEPTCVSDYMSISTCEWKMGGPTNCSAELRLLYQLVFQSSETHTCVPENNGGVGCVCHLLMDDVVSMDNYTLDLWAGQQLLWKGSFKPSEHVKPRAPGNLTVHTNVSDTVLLTWSNPYPPDNYLYNDLTYAVNIWSENDPAYSRIHNVTYLKPTLRIPASTLKSGISYRARVRAWAQHYNTTWSEWSPSTKWYNSYREPFEQR

SEQ ID NO: 17 - protein, 153 aa, Homo sapiens [sequence omitted]

SEQ ID NO: 18 - protein, 146 aa, Homo sapiens [sequence omitted]

SEQ ID NO: 19 - protein, 13 aa, synthetic construct
AASGFTFSISSMN

SEQ ID NO: 20 - protein, 10 aa, synthetic construct
YISRATGAIY

SEQ ID NO: 21 - skipped

SEQ ID NO: 22 - protein, 11 aa, synthetic construct
RASQGISNYLA

SEQ ID NO: 23 - skipped

SEQ ID NO: 24 - protein, 9 aa, synthetic construct
LQHNSYPRT

SEQ ID NO: 25 - protein, 115 aa, synthetic construct
QVQLVESGGGLVQPGGSLRLSCAASGFTFSISSMNWVRQAPGKGLEWVSYISRATGAIYYADSVKGRFTISRNNAKNSLYLQMNSLRDEDTAVYYCAREPVFDYWGQGTLVTVSS

SEQ ID NO: 26 - protein, 107 aa, synthetic construct
DIQMTQSPSAMSASVGDRVTITCRASQGISNYLAWFQQKPGKVPTRLIYAASSLQSGVPSRFSGSGSGTEFTLTISSLQPEDFATYYCLQHNSYPRTFGQGTKVEIK

SEQ ID NO: 27 - protein, 441 aa, synthetic construct
QVQLVESGGGLVQPGGSLRLSCAASGFTFSISSMNWVRQAPGKGLEWVSYISRATGAIYYADSVKGRFTISRNNAKNSLYLQMNSLRDEDTAVYYCAREPVFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 28 - protein, 214 aa, synthetic construct
DIQMTQSPSAMSASVGDRVTITCRASQGISNYLAWFQQKPGKVPTRLIYAASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 1323 bp, synthetic construct
  source 1..1323: mol_type = other DNA
caggtacagctggtggagtctgggggaggcttggtacagcctggggggtccctgagactctcctgtgcagcctctggattcaccttcagtatctctagcatgaactgggtccgccaggctccagggaaggggctggagtgggtttcatacattagtcgtgctactggtgccatatactacgcagactctgtaaagggccgattcaccatctccagaaacaatgccaaaaactcactgtatctgcaaatgaacagcctgagagacgaggacacggctgtgtattactgtgcgagagagccggtttttgactactggggccagggaaccctggtcaccgtctcctcagcttctaccaagggcccatcggtcttcccgctagcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 30 - DNA, 642 bp, synthetic construct
  source 1..642: mol_type = other DNA
gacatccagatgacccagtctccatctgccatgtctgcatctgtgggagacagagtcaccatcacttgtcgggcgagtcagggcattagcaattatttagcctggtttcagcagaaaccagggaaagtccctacgcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttaccctcggacgttcggccaagggaccaaggtggaaatcaaacgaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 31 - protein, 441 aa, synthetic construct
QVQLVESGGGLVQPGGSLRLSCVASGFTFSHSSMNWVRQAPGKGLEWVSYISRATGAVYYADSVKGRFTISRDNAKNSLYLQMNSLRDEDTAVYYCAREPVFDYWGQGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDICVEWESNGQPENNYKTTPPVLDSDGSFFLYSRLTVDKSRWQEGNVFSCSVMHEALHNHYTQKSLSLSLG

SEQ ID NO: 32 - DNA, 1323 bp, synthetic construct
  source 1..1323: mol_type = other DNA
caggtacagctggtggagtctgggggaggcttggtacagcctggggggtccctgagactctcctgtgtcgcctctggattcaccttcagtcattctagcatgaactgggtccgccaggctccagggaaggggctggagtgggtttcatacattagtcgtgctactggtgccgtctactacgcagactctgtaaagggccgattcaccatctccagagataatgccaaaaactcactgtatctgcaaatgaacagcctgagagacgaggacacggctgtgtattactgtgcgagagagccggtttttgactactggggccagggaaccctggtcaccgtctcctcagcctccaccaagggcccatgcgtcttcccgctagcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatctgcgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 33 - protein, 445 aa, synthetic construct
QVQLVESGGGLVQPGGSLRLSCVASGFTFSHSSMNWVRQAPGKGLEWVSYISRATGAVYYADSVKGRFTISRDNAKNSLYLQMNSLRDEDTAVYYCAREPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 1335 bp, synthetic construct
  source 1..1335: mol_type = other DNA
caggtacagctggtggagtctgggggaggcttggtacagcctggggggtccctgagactctcctgtgtcgcctctggattcaccttcagtcattctagcatgaactgggtccgccaggctccagggaaggggctggagtgggtttcatacattagtcgtgctactggtgccgtctactacgcagactctgtaaagggccgattcaccatctccagagataatgccaaaaactcactgtatctgcaaatgaacagcctgagagacgaggacacggctgtgtattactgtgcgagagagccggtttttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcgccgtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 35 - protein, 441 aa, synthetic construct
QVQLVESGGGLVQPGGSLRLSCVASGFTFSHSSMNWVRQAPGKGLEWVSYISRATGAVYYADSVKGRFTISRDNAKNSLYLQMNSLRDEDTAVYYCAREPVFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 36 - DNA, 1323 bp, synthetic construct
  source 1..1323: mol_type = other DNA
caggtacagctggtggagtctgggggaggcttggtacagcctggggggtccctgagactctcctgtgtcgcctctggattcaccttcagtcattctagcatgaactgggtccgccaggctccagggaaggggctggagtgggtttcatacattagtcgtgctactggtgccgtctactacgcagactctgtaaagggccgattcaccatctccagagataatgccaaaaactcactgtatctgcaaatgaacagcctgagagacgaggacacggctgtgtattactgtgcgagagagccggtttttgactactggggccagggaaccctggtcaccgtctcctcagcctccaccaagggcccatcggtcttcccgctagcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtcaagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccgagaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcagcaggggaatgtcttctcatgctccgtgatgcatgaggctctgcacaaccactacacacagaagagcctctccctgtctctgggt

SEQ ID NO: 37 - protein, 441 aa, synthetic construct
QVQLVESGGGLVQPGGSLRLSCVASGFTFSHSSMNWVRQAPGKGLEWVSYISRATGAVYYADSVKGRFTISRDNAKNSLYLQMNSLRDEDTAVYYCAREPVFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 38 - DNA, 1323 bp, synthetic construct
  source 1..1323: mol_type = other DNA
caggtacagctggtggagtctgggggaggcttggtacagcctggggggtccctgagactctcctgtgtcgcctctggattcaccttcagtcattctagcatgaactgggtccgccaggctccagggaaggggctggagtgggtttcatacattagtcgtgctactggtgccgtctactacgcagactctgtaaagggccgattcaccatctccagagataatgccaaaaactcactgtatctgcaaatgaacagcctgagagacgaggacacggctgtgtattactgtgcgagagagccggtttttgactactggggccagggaaccctggtcaccgtctcctcagcctccaccaagggcccatcggtcttcccgctagcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 39 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 40 - protein, 832 aa, Macaca fascicularis
MGWLCSGLLFPVSCLVLLQVASSGCSCVSPGSMKVLQEPTCVSDYMSISTCEWKMGGPTNCSAELRLLYQLVFQSSETHTCVPENNGGVGCVCHLLMDDVVSMDNYTLDLWAGQQLLWKGSFKPSEHVKPRAPGNLTVHTNVSDTVLLTWSNPYPPDNYLYNDLTYAVNIWSENDPAYSRIHNVTYLKPTLRIPASTLKSGISYRARVRAWAQHYNTTWSEWSPSTKWYNSYREPFEQRLLWGVSAACVFILFFCLSCYFSVTKIKKEWWDQIPNPARSHLVAIIIQDAQESQWEKRSRGQEAAKCPYWKNCLTKLLPCFLEHNMKRDEDPHKAVKDLPFRGSGKSAWCPVEISKTVLWPESISVVRCVELFEAPVECKEEEEVEEEKGSFCTSSESNRDDFQEGREGIVARLTESLFLDLLGGENGGFFQQDMGESCLLPPLGSTSAHVPWDEFPSAGSKEVPPWGKEQPLHQEPSPPASPTQSPDNPTCTEMPLVISSNPAYRSFSNSLSQSPCPRELGPDPLLARHLEEVDPEMPCAPQLSEPTTVAPAEPETWEQILRRNVLQHGAAAAPASAPTSGYREFVHAVQQGGIQASAVAGLGPPGEAGYKAFSSLLASSAVSPGECGFGASSGEEGYKPFQDLTPGCPGDPAPVPVPLFTFGLDREPPHSPQSSHLPSNSPEHLALEPGEKVEDMQKPPLPPEQATDPLGDSLGSGIVYSALTCHLCGHLKQCHGQEDGGQAPVVASPCCGCCCGDRSSPPTTPLRAPDPSLGGVPLEASLCPASLAPSGISEKSKSSLSFHPAPGSAQSSSQTPQIVNFVSVGPTCMRVS

SEQ ID NO: 41 - protein, 321 aa, Homo sapiens
MEEGQYSEIEELPRRRCCRRGTQIVLLGLVTAALWAGLLTLLLLWHWDTTQSLKQLEERAARNVSQVSKNLESHHGDQMAQKSQSTQISQELEELRAEQQRLKSQDLELSWNLNGLQADLSSFKSQELNERNEASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAESMGPDSRPDPDGRLPTPSAPLHS

SEQ ID NO: 42 - protein, 13 aa, synthetic construct
AASGFTFSHSSMN

SEQ ID NO: 43 - skipped

SEQ ID NO: 44 - protein, 115 aa, synthetic construct
QVQLVESGGGLVQPGGSLRLSCAASGFTFSHSSMNWVRQAPGKGLEWVSYISRATGAVYYADSVKGRFTISRDNAKNSLYLQMNSLRDEDTAVYYCAREPVFDYWGQGTLVTVSS

SEQ ID NO: 45 - protein, 107 aa, synthetic construct
DIQMTQSPSAMSASVGDRVTITCRASQGISNYLAWFQQKPGKVPTRLIYAASSLQSGVPSRFSGSGSGTEFTLTISSLQPEDFATYYCLQWSSYPRTFGQGTKVEIK

SEQ ID NO: 46 - protein, 441 aa, synthetic construct
QVQLVESGGGLVQPGGSLRLSCAASGFTFSHSSMNWVRQAPGKGLEWVSYISRATGAVYYADSVKGRFTISRDNAKNSLYLQMNSLRDEDTAVYYCAREPVFDYWGQGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDICVEWESNGQPENNYKTTPPVLDSDGSFFLYSRLTVDKSRWQEGNVFSCSVMHEALHNHYTQKSLSLSLG

SEQ ID NO: 47 - protein, 214 aa, synthetic construct
DIQMTQSPSAMSASVGDRVTITCRASQGISNYLAWFQQKPGKVPTRLIYAASSLQSGVPSRFSGSGSGTEFTLTISSLQPEDFATYYCLQWSSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - DNA, 1323 bp, synthetic construct
  source 1..1323: mol_type = other DNA
caggtacagctggtggagtctgggggaggcttggtacagcctggggggtccctgagactctcctgtgcagcctctggattcaccttcagtcattctagcatgaactgggtccgccaggctccagggaaggggctggagtgggtttcatacattagtcgtgctactggtgccgtctactacgcagactctgtaaagggccgattcaccatctccagagataatgccaaaaactcactgtatctgcaaatgaacagcctgagagacgaggacacggctgtgtattactgtgcgagagagccggtttttgactactggggccagggaaccctggtcaccgtctcctcagcctccaccaagggcccatgcgtcttcccgctagcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatctgcgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 49 - DNA, 642 bp, synthetic construct
  source 1..642: mol_type = other DNA
gacatccagatgacccagtctccatctgccatgtctgcatctgtgggagacagagtcaccatcacttgtcgggcgagtcagggcattagcaattatttagcctggtttcagcagaaaccagggaaagtccctacgcgcctgatctatgctgcatccagtttgcaaagtggggtcccatcaaggttcagcggcagtggatctgggacagaattcactctcacaatcagcagcctgcagcctgaagattttgcaacttattactgtctacagtggtccagttaccctcggacgttcggccaagggaccaaggtggaaatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 50 - protein, 441 aa, synthetic construct
QVQLVESGGGLVQPGGSLRLSCVASGFTFSHSSMNWVRQAPGKGLEWVSYISRATGAVYYADSVKGRFTISRDNAKNSLYLQMNSLRDEDTAVYYCAREPVFDYWGQGTLVTVSSASTKGPCVFPLAPCSRSTSGSTAALGCLVKDYFPEPVTVSWNSGALTSGVHTFPAVLQSSGLYSLSSVVTVPSSSLGTKTYTCNVNHKPSNTKVDKRVESKYGPPCPPCPAPEFLGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDICVEWESNGQPENNYKTTPPVLDSDGSFFLYSRLTVDKSRWQQGNVFSCSVMHEALHNHYTQKSLSLSLG

SEQ ID NO: 51 - DNA, 1323 bp, synthetic construct
  source 1..1323: mol_type = other DNA
caggtacagctggtggagtctgggggaggcttggtacagcctggggggtccctgagactctcctgtgtcgcctctggattcaccttcagtcattctagcatgaactgggtccgccaggctccagggaaggggctggagtgggtttcatacattagtcgtgctactggtgccgtctactacgcagactctgtaaagggccgattcaccatctccagagataatgccaaaaactcactgtatctgcaaatgaacagcctgagagacgaggacacggctgtgtattactgtgcgagagagccggtttttgactactggggccagggaaccctggtcaccgtctcctcagcctccaccaagggcccatgcgtcttcccgctagcgccctgctccaggagcacctccggcagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtaaaccacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtcaagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccgagaggagatgaccaagaaccaggtcagcctgacctgcctggtcaaaggcttctaccccagcgacatctgcgtggagtgggaaagcaatgggcagccggagaacaactacaagaccacgcctcccgtgctggactccgacggctccttcttcctctacagcaggctaaccgtggacaagagcaggtggcagcaggggaatgtcttctcatgctccgtgatgcatgaggctctgcacaaccactacacacagaagagcctctccctgtctctgggt

SEQ ID NO: 52 - protein, 445 aa, synthetic construct
QVQLVESGGGLVQPGGSLRLSCVASGFTFSHSSMNWVRQAPGKGLEWVSYISRATGAVYYADSVKGRFTISRDNAKNSLYLQMNSLRDEDTAVYYCAREPVFDYWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDICVEWESNGQPENNYKTTPPVLDSDGSFFLYSKLTVDKSRWQQGNVFSCSVMHEALHNHYTQKSLSLSPGK

SEQ ID NO: 53 - DNA, 1335 bp, synthetic construct
  source 1..1335: mol_type = other DNA
caggtacagctggtggagtctgggggaggcttggtacagcctggggggtccctgagactctcctgtgtcgcctctggattcaccttcagtcattctagcatgaactgggtccgccaggctccagggaaggggctggagtgggtttcatacattagtcgtgctactggtgccgtctactacgcagactctgtaaagggccgattcaccatctccagagataatgccaaaaactcactgtatctgcaaatgaacagcctgagagacgaggacacggctgtgtattactgtgcgagagagccggtttttgactactggggccagggaaccctggtcaccgtctcctcagctagcaccaagggcccatgcgtcttccccctggcaccctcctccaagagcacctctgggggcacagcggccctgggctgcctggtcaaggactacttccccgaaccggtgacggtgtcgtggaactcaggcgcactgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcgccgcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatctgcgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

(19) United States
(12) Patent Application Publication
(10) Pub. No.: US 2024/0100176 A1
(43) Pub. Date: Mar. 28, 2024

(54) HUMAN INTERLEUKIN-4 RECEPTOR ALPHA ANTIBODY GLUCOCORTICOID CONJUGATES

(71) Applicant: Eli Lilly and Company, Indianapolis, IN (US)

(72) Inventors: Shane Krummen Atwell, Carlsbad, CA (US); Joshua R Clayton, Fishers, IN (US); Yiqing Feng, Carmel, IN (US); Maya Rachel Karta, San Diego, CA (US); Donmienne Doen Mun Leung, Saltburn-by-the-sea (GB); Songqing Na, San Diego, CA (US); Kristin Paige Newburn, Indianapolis, IN (US); Laura Anne Pelletier, San Diego, CA (US); Diana Isabel Ruiz, San Diego, CA (US); David John Stokell, Indianapolis, IN (US); Jacqueline M Wurst, Zionsville, IN (US); Scott Paul Bauer, Bloomington, IN (US)

(21) Appl. No.: 18/326,461

(22) Filed: May 31, 2023

Related U.S. Application Data
(60) Provisional application No. 63/348,197, filed on Jun. 2, 2022.

(51) Int. Cl. A61K 47/68; A61K 47/55

(52) U.S. Cl. CPC A61K 47/6803; A61K 47/55; A61K 47/6849

(57) ABSTRACT

The present disclosure provides human interleukin 4 receptor alpha antibody glucocorticoid receptor agonist conjugates and methods of using the conjugates for the treatment of inflammatory diseases, such as type 2 inflammatory diseases.

### SEQUENCE LISTING FILE

The present application is being filed along with a Sequence Listing in ST.26 XML format. The Sequence Listing is provided as a file titled “30167_US_Sequence_ListingFINAL” created Mar. 30, 2023, and is 67 kilobytes in size. The Sequence Listing information in the ST.26 XML format is incorporated herein by reference in its entirety.

### FIELD OF THE INVENTION

The present disclosure provides human interleukin-4 receptor alpha antibody glucocorticoid receptor agonist conjugates, methods of using the conjugates for the treatment of inflammatory diseases, such as Type 2 inflammatory diseases such as atopic dermatitis, eosinophilic esophagitis, nasal polyposis, asthma, chronic rhinosinusitis, allergic disease, chronic obstructive pulmonary disease, or chronic spontaneous urticaria, processes for preparing the conjugates, and pharmaceutical compositions comprising the conjugates.

### BACKGROUND OF THE INVENTION

Atopic dermatitis is a chronic, pruritic relapsing and remitting inflammatory skin disease that occurs frequently in children, but also affects many adults. Current treatments of atopic dermatitis include light therapy, topical creams containing corticosteroids or calcineurin inhibitors, or a subcutaneous injectable biologic known as dupilumab. In spite of progress made in treating atopic dermatitis, there remains a significant need for new compounds to treat atopic dermatitis and other inflammatory and autoimmune diseases, and which minimize or eliminate disadvantages possessed by currently approved treatments.

WO2017/210471 discloses certain glucocorticoid receptor agonists (GC) and immunoconjugates thereof useful for treating inflammatory diseases. WO2018/089373 discloses novel steroids, protein conjugates thereof, and methods for treating diseases, disorders, and conditions comprising administering the steroids and conjugates. To date, there are no known human IL-4Rα GC conjugates for the treatment of inflammatory diseases in development.

### SUMMARY OF THE INVENTION

The present disclosure provides certain novel human IL-4Rα antibody GC conjugates, wherein the antibody binds to human IL-4Rα. The present disclosure further provides compositions comprising novel anti-human IL-4Rα antibody GC conjugates and methods of using such anti-human IL-4Rα antibody GC conjugates and compositions thereof. The present disclosure further provides certain novel anti-human IL-4Rα GC conjugates useful in the treatment of inflammatory diseases such as Type 2 inflammatory diseases such as atopic dermatitis, eosinophilic esophagitis, nasal polyposis, asthma, chronic rhinosinusitis, allergic disease, chronic obstructive pulmonary disease, or chronic spontaneous urticaria.

Certain anti-human IL-4Rα antibody GC conjugates provided herein have one or more of the following properties: 1) bind a novel epitope spanning the n-terminal fibronectin type-III domains 1 and 2 of the human and/or cynomolgus monkey IL-4Rα, 2) bind human IL-4Rα with desirable binding affinities, 3) bind cynomolgus monkey IL-4Rα with desirable binding affinities, 4) bind human IL-4Rα on the cell surface and internalize into the cell, 5) bind human IL-4Rα and inhibit IL-4 and IL-13 binding to IL-4Rα, 6) bind IL-4Rα and inhibit IL-4R mediated responses (pSTAT6 in B and T cells, B cell proliferation, CD23 expression, cytokine production (e.g., MDC, GM-CSF)) in vitro, 7) bind a unique functional epitope on the human and/or cynomolgus monkey IL-4Rα, 8) modulate glucocorticoid receptor agonist mediated responses (induce CD163 expression, inhibit B cell proliferation, inhibit cytokine production (e.g., IL-5, GM-CSF)) in vitro, 9) modulate glucocorticoid receptor agonist mediated gene expression (upregulate: Tsc22d3, Fkbp5, Zbtb16; downregulate: IL-5), 10) do not significantly induce ADCC or CDC activity, 11) retain Fcγ receptor binding to B cells and/or myeloid cells, 12) inhibit inflammatory responses in vivo, or 13) have a good developability profile e.g., acceptable viscosity, solubility and aggregation stability to facilitate development, manufacturing, and/or formulation.

Accordingly, in one embodiment, the disclosure provides a conjugate of Formula I:

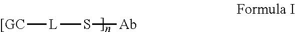

wherein Ab is an antibody that binds human interleukin-4 receptor alpha (“anti-human IL-4Rα antibody”), and wherein

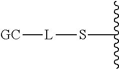

is:

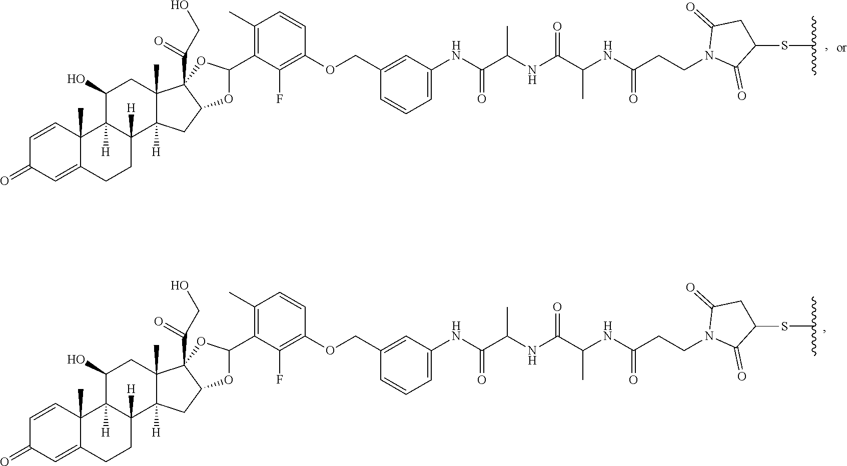

and n is 1-5.

In some embodiments, the Ab in conjugate of Formula I binds to an epitope of human IL-4Rα, wherein the epitope comprises one or more amino acid residues selected from D12, M14, S15, I16, Y37, L39, F41, L42, L43, E45, H47, T48, C49, 150, E52, H62, L64, M65, D66, D67, V68, V69, D72, R99, P121, P123, P124, D125, P192 (the amino acid residue positions correspond to SEQ ID NO: 15). In some embodiments, the Ab in conjugate of Formula I binds to an epitope of human IL-4Rα, wherein the epitope comprises at least one, at least two, at least three, at least four, or at least five or more amino acid residues selected from D12, M14, S15, I16, Y37, L39, F41, L42, L43, E45, H47, T48, C49, I50, E52, H62, L64, M65, D66, D67, V68, V69, D72, R99, P121, P123, P124, D125, P192 (the amino acid residue positions correspond to SEQ ID NO: 15). In some embodiments, the Ab in conjugate of Formula I binds to an epitope of human IL-4Rα, wherein the epitope comprises one or more amino acid residues selected from D12, M14, S15, I16, L39, F41, L42, T48, C49, I50, E52, H62, L64, M65, D66, D67, V68, V69, D72, R99, P121, P123, P124, D125, P192 (the amino acid residue positions correspond to SEQ ID NO: 15). In some embodiments, the Ab in conjugate of Formula I binds to an epitope of human IL-4Rα, wherein the epitope comprises at least one, at least two, at least three, at least four, at least five or more amino acid residues selected from D12, M14, S15, I16, L39, F41, L42, T48, C49, 150, E52, H62, L64, M65, D66, D67, V68, V69, D72, R99, P121, P123, P124, D125, P192 (the amino acid residue positions correspond to SEQ ID NO: 15). In some embodiments, the Ab in conjugate of Formula I binds to an epitope of human IL-4Rα, wherein the epitope comprises one or more amino acid residues selected from D12, M14, S15, I16, Y37, L39, F41, L43, E45, H47, T48, C49, I50, H62, L64, M65, D66, D67, V69, D72, R99, P121, P123, P124, D125 (the amino acid residue positions correspond to SEQ ID NO: 15). In some embodiments, the Ab in conjugate of Formula I binds to an epitope of human IL-4Rα, wherein the epitope comprises at least one, at least two, at least three, at least four, at least five or more amino acid residues selected from D12, M14, S15, I16, Y37, L39, F41, L43, E45, H47, T48, C49, I50, H62, L64, M65, D66, D67, V69, D72, R99, P121, P123, P124, D125 (the amino acid residue positions correspond to SEQ ID NO: 15). In some embodiments, the Ab in conjugate of Formula I binds to an epitope of human IL-4Rα, wherein the epitope comprises one or more amino acid residues selected from D12, M14, S15, I16, Y37, L39, T48, C49, I50, E52, H62, M65, R99, P121, P123, P124, D125, P192 (the amino acid residue positions correspond to SEQ ID NO: 15). In some embodiments, the Ab in conjugate of Formula I binds to an epitope of human IL-4Rα, wherein the epitope comprises at least one, at least two, at least three, at least four, at least five or more amino acid residues selected from D12, M14, S15, I16, Y37, L39, T48, C49, I50, E52, H62, M65, R99, P121, P123, P124, D125, P192 (the amino acid residue positions correspond to SEQ ID NO: 15). In some embodiments, the Ab in conjugate of Formula I binds to an epitope of human IL-4Rα, wherein the epitope comprises one or more amino acid residue selected from R99, P121, P123, P124, D125, P192 (the amino acid residue positions correspond to SEQ ID NO: 15), wherein these residues are located in domain 2 of the N-terminal fibronectin type-III domains of the IL-4Rα. In some embodiments, the Ab in conjugate of Formula I binds to an epitope of human IL-4Rα, wherein the epitope comprises one or more amino acid residue selected from D66, D67, and D125 (the amino acid residue positions correspond to SEQ ID NO: 15). In some embodiments, the Ab in conjugate of Formula I binds to an epitope of human IL-4Rα, wherein the epitope comprises at least one of amino acid residues selected from D66 and D67 (the amino acid residue positions correspond to SEQ ID NO: 15). In some embodiments, the Ab in conjugate of Formula I binds to an epitope of human IL-4Rα, wherein the epitope comprises at least one of amino acid residues selected from D66 and D125 (the amino acid residue positions correspond to SEQ ID NO: 15). In some embodiments, the Ab in conjugate of Formula I binds to an epitope of human IL-4Rα, wherein the epitope comprises amino acid residue D66 (the amino acid residue positions correspond to SEQ ID NO: 15). In yet other embodiments, the Ab in conjugate of Formula I binds a novel structural and/or functional epitope of the human IL-4Rα, wherein the epitope spans domain 1 and domain 2 of the n-terminal fibronectin type-III domains of the IL-4Rα. In particular embodiments, the Ab in conjugate of Formula I, binds a novel structural and/or functional epitope of the human IL-4Rα, wherein the epitope overlaps with the IL-4 binding site to IL-4Rα. In such embodiments, the Ab in conjugate of Formula I blocks binding of IL-4 to the human IL-4Rα. In particular embodiments, the Ab in conjugate of Formula I, binds a novel structural and/or functional epitope of the human IL-4Rα, wherein the epitope overlaps with the IL-13 binding site to IL-4Rα. In such embodiments, the Ab in conjugate of Formula I blocks binding of IL-13 to the human IL-4Rα. In particular embodiments, the Ab in conjugate of Formula I, binds a novel structural and/or functional epitope of the human IL-4Rα, wherein the epitope overlaps with both the IL-4 and the IL-13 binding sites to IL-4Rα. In such embodiments, the Ab in conjugate of Formula I blocks binding of IL-4 and IL-13 to the human IL-4Rα. In some embodiments, the conjugate of Formula I, wherein the IL-4Rα epitope is determined by X-ray crystallography, alanine scanning mutagenesis, steric hindrance mutagenesis, and/or HDX-MS. In yet other embodiments, the IL-4Rα epitope is determined by site-directed mutagenesis.

In a further embodiment, the disclosure provides a conjugate of Formula I:

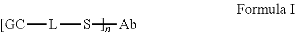

wherein Ab is an antibody that binds human interleukin-4 receptor alpha (“anti-human IL-4Rα antibody”), wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1, 19, or 42;
the HCDR2 comprises SEQ ID NO: 2 or 20;
the HCDR3 comprises SEQ ID NO: 3;
the LCDR1 comprises SEQ ID NO: 4 or 22;
the LCDR2 comprises SEQ ID NO: 5; and
the LCDR3 comprises SEQ ID NO: 6 or 24;
   and wherein

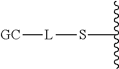

is:

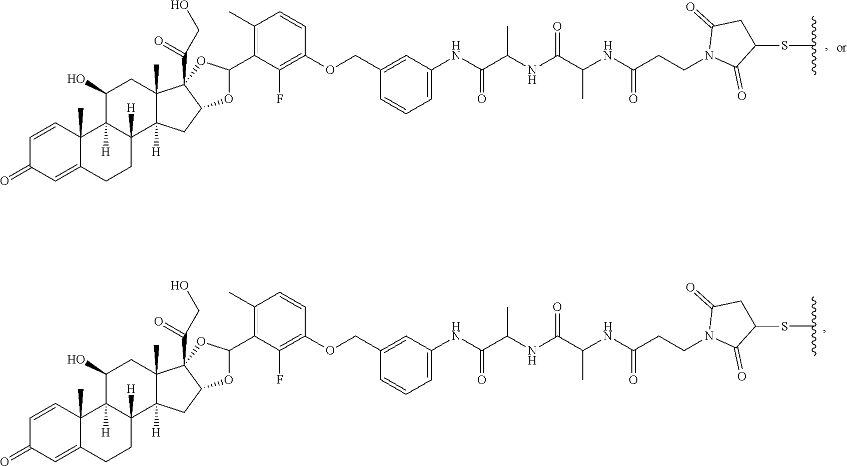

and n is 1-5.

In a further embodiment, the disclosure provides a conjugate of Formula Ia:

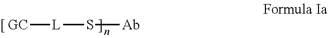

wherein Ab is an antibody that binds human IL-4Rα, wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1, 19, or 42;
the HCDR2 comprises SEQ ID NO: 2 or 20;
the HCDR3 comprises SEQ ID NO: 3;
the LCDR1 comprises SEQ ID NO: 4 or 22;
the LCDR2 comprises SEQ ID NO: 5; and
the LCDR3 comprises SEQ ID NO: 6 or 24;
   and wherein

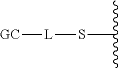

is:

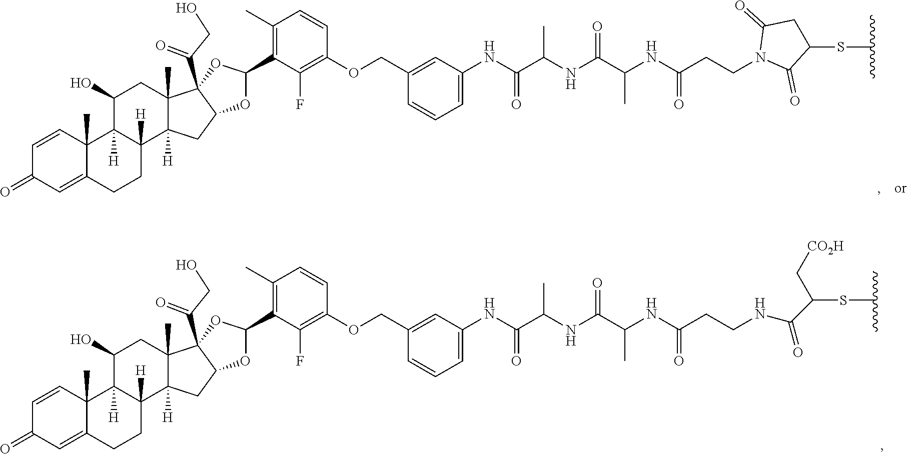

and n is 1-5.

In a further embodiment, the disclosure provides a conjugate of Formula Ib:

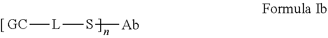

wherein Ab is an antibody that binds human IL-4Rα, wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1, 19, or 42;
the HCDR2 comprises SEQ ID NO: 2 or 20;
the HCDR3 comprises SEQ ID NO: 3;
the LCDR1 comprises SEQ ID NO: 4 or 22;
the LCDR2 comprises SEQ ID NO: 5; and
the LCDR3 comprises SEQ ID NO: 6 or 24;
   and wherein

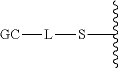

is:

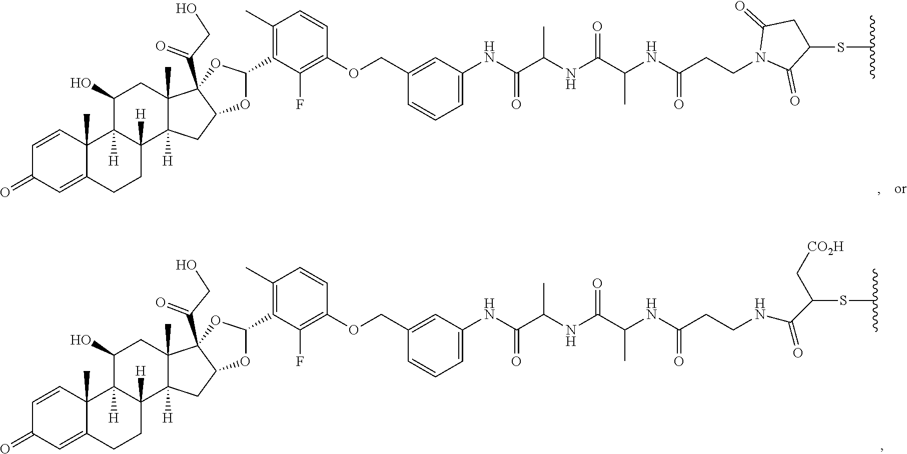

and n is 1-5.

In a further embodiment, the disclosure provides a conjugate of Formula Ic:

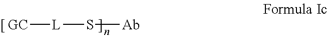

wherein Ab is an antibody that binds human IL-4Rα, wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1, 19, or 42;
the HCDR2 comprises SEQ ID NO: 2 or 20;
the HCDR3 comprises SEQ ID NO: 3;
the LCDR1 comprises SEQ ID NO: 4 or 22;
the LCDR2 comprises SEQ ID NO: 5; and
the LCDR3 comprises SEQ ID NO: 6 or 24;
   and wherein

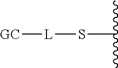

is:

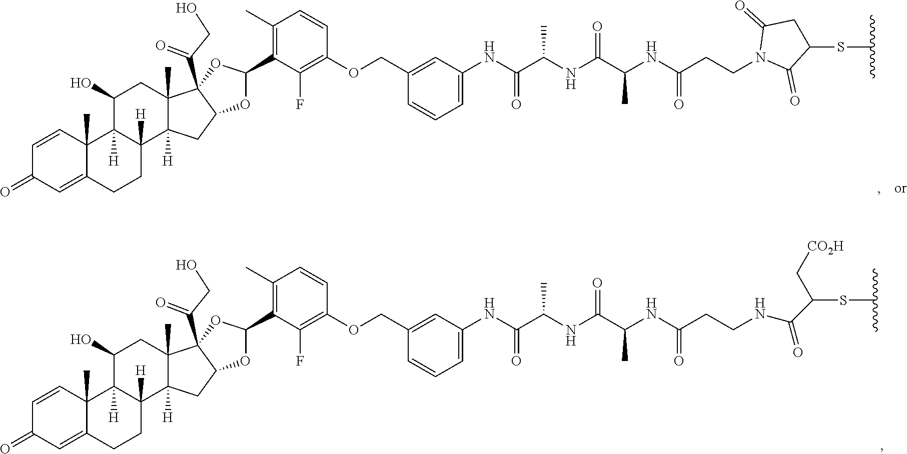

and n is 1-5.

In a further embodiment, the disclosure provides a conjugate of Formula Id:

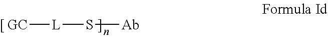

wherein Ab is an antibody that binds human IL-4Rα, wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1, 19, or 42;
the HCDR2 comprises SEQ ID NO: 2 or 20;
the HCDR3 comprises SEQ ID NO: 3;
the LCDR1 comprises SEQ ID NO: 4 or 22;
the LCDR2 comprises SEQ ID NO: 5; and
the LCDR3 comprises SEQ ID NO: 6 or 24;
and wherein

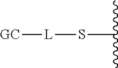

is:

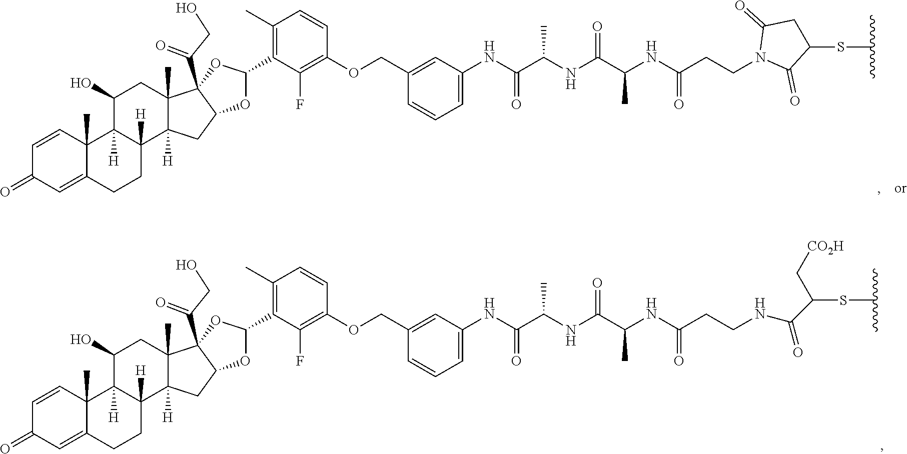

and n is 1-5.

In a further embodiment, the disclosure provides a conjugate of Formula Ie:

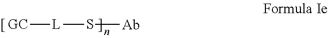

wherein Ab is an antibody that binds human IL-4Rα, wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1, 19, or 42;
the HCDR2 comprises SEQ ID NO: 2 or 20;
the HCDR3 comprises SEQ ID NO: 3;
the LCDR1 comprises SEQ ID NO: 4 or 22;
the LCDR2 comprises SEQ ID NO: 5; and
the LCDR3 comprises SEQ ID NO: 6 or 24;
and wherein

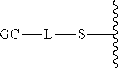

is:

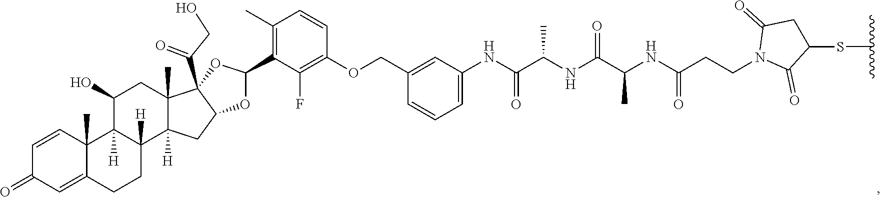

and n is 1-5.

In a further embodiment, the disclosure provides a conjugate of Formula If:

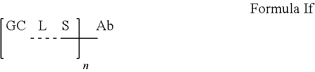

wherein Ab is an antibody that binds human IL-4Rα, wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1, 19, or 42;
the HCDR2 comprises SEQ ID NO: 2 or 20;
the HCDR3 comprises SEQ ID NO: 3;
the LCDR1 comprises SEQ ID NO: 4 or 22;
the LCDR2 comprises SEQ ID NO: 5; and
the LCDR3 comprises SEQ ID NO: 6 or 24;
   and wherein

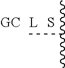

is:

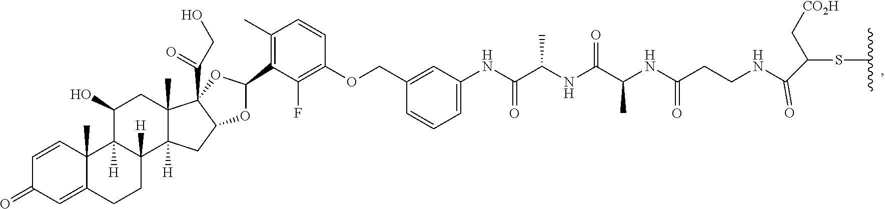

and n is 1-5.

In an embodiment, n is 2-5.

In an embodiment, n is 3-5.

In an embodiment, n is 3-4.

In an embodiment, n is about 4.

In an embodiment, n is about 3.

In an embodiment, n is about 2.

In some embodiments, the Ab comprises a VH and a VL, wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 1, the HCDR2 comprises SEQ ID NO: 2, the HCDR3 comprises SEQ ID NO: 3, the LCDR1 comprises SEQ ID NO: 4, the LCDR2 comprises SEQ ID NO: 5, and the LCDR3 comprises SEQ ID NO: 6. In some embodiments, the Ab comprises a VH comprising SEQ ID NO: 7 and a VL comprising SEQ ID NO: 8. In some embodiments, the Ab is Ab1, wherein Ab1 comprises a HC comprising SEQ ID NO: 9 and a LC comprising SEQ ID NO: 10. In some embodiments, the Ab is Ab2, wherein Ab2 comprises a HC comprising SEQ ID NO: 50 and a LC comprising SEQ ID NO: 10. In some embodiments, the Ab is Ab3, wherein Ab3 comprises a HC comprising SEQ ID NO: 37 and a LC comprising SEQ ID NO: 10. In some embodiments, the Ab is Ab4, wherein Ab4 comprises a HC comprising SEQ ID NO: 31 and a LC comprising SEQ ID NO: 10. In some embodiments, the Ab is Ab5, wherein Ab5 comprises a HC comprising SEQ ID NO: 35 and a LC comprising SEQ ID NO: 10. In some embodiments, the Ab is Ab6, wherein Ab6 comprises a HC comprising SEQ ID NO: 33 and a LC comprising SEQ ID NO: 10. In some embodiments, the Ab is Ab7, wherein Ab7 comprises a HC comprising SEQ ID NO: 13 and a LC comprising SEQ ID NO: 10. In some embodiments, the Ab is Ab8, wherein Ab8 comprises a HC comprising SEQ ID NO: 52 and a LC comprising SEQ ID NO: 10.

In some embodiments, the Ab comprises a VH and a VL, wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 42, the HCDR2 comprises SEQ ID NO: 2, the HCDR3 comprises SEQ ID NO: 3, the LCDR1 comprises SEQ ID NO: 22, the LCDR2 comprises SEQ ID NO: 5, and the LCDR3 comprises SEQ ID NO: 6. In some embodiments, the Ab comprises a VH comprising SEQ ID NO: 44 and a VL comprising SEQ ID NO: 45. In some embodiments, the Ab is Ab9, wherein Ab9 comprises a HC comprising SEQ ID NO: 46 and a LC comprising SEQ ID NO: 47.

In some embodiments, the Ab comprises a VH and a VL, wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 19, the HCDR2 comprises SEQ ID NO: 20, the HCDR3 comprises SEQ ID NO: 3, the LCDR1 comprises SEQ ID NO: 22, the LCDR2 comprises SEQ ID NO: 5, and the LCDR3 comprises SEQ ID NO: 24. In some embodiments, the Ab comprises a VH comprising SEQ ID NO: 25 and a VL comprising SEQ ID NO: 26. In some embodiments, the Ab is Ab10, wherein Ab10 comprises a HC comprising SEQ ID NO: 27 and a LC comprising SEQ ID NO: 28.

In some embodiments, the Ab in conjugate of Formula I binds human IL-4Rα and inhibits binding of human IL-4 and human IL-13 to human IL-4Rα. In some embodiments, the Ab in conjugate of Formula I binds human IL-4Rα and inhibits IL-4R receptor mediated responses (e.g., inhibits pSTAT6 in B and T cells, inhibits B cell proliferation, inhibits CD23 expression, inhibits cytokine secretion (e.g., MDC, GM-CSF). In some embodiments, the Ab in conjugate of Formula I binds human IL-4Rα and does not significantly induce ADCC or CDC activity. In some embodiments, the Ab in conjugate of Formula I binds human IL-4Rα on a cell surface and internalizes the conjugate of Formula I into the cell.

In some embodiments of the present disclosure, the Ab in conjugate of Formula I is a fully human antibody. In some embodiments of the present disclosure, the Ab in conjugate of Formula I has a human IgG4 or a human IgG1 isotype.

In some embodiments of the present disclosure, the Ab in conjugate of Formula I has a modified human IgG4 hinge region comprising a S228P substitution (EU Numbering), also referred to as IgG4P, which reduces the IgG4 Fab-arm exchange in vivo (see Labrijn, et al., Nat. Biotechnol. 2009, 27(8):767). In some embodiments, the Ab having a human IgG4P backbone has improved binding to B cells and/or myeloid cells, when compared to a human IgG4 and/or human IgG4P IL-4Rα antibody having an effector null backbone.

In some embodiments of the present disclosure, the Ab in conjugate of Formula I has a modified human IgG1 Fc region comprising an alanine at amino acid residue 322 (K322A substitution) (EU numbering) (also referred to as IgG1A). In such embodiments, the Ab has reduced or eliminated complement activity. In some embodiments, the Ab has a modified human IgG1 Fc region comprising a L234A, an L235A and/or a P329A (also referred to as IgG1AA or IgGIAAA respectively), which have reduced or eliminated binding to the Fcγ and C1q receptors (all residues numbered according to EU numbering). In some embodiments, the Ab having a human IgG1A backbone shows improved binding to B cells and/or myeloid cells, when compared to the anti-human IL-4Rα antibody having a human IgG1AAA effector null backbone.

In some embodiments of the present disclosure, the Ab in conjugate of Formula I has a modified human IgG4 HC constant region which reduces viscosity of the antibody compared to the same antibody with a wild-type human IgG4 HC constant region. In such embodiments, the Ab has a modified human IgG4 HC constant region comprising an amino acid substitution at any one or more of the following amino acid residues: E137G, D203N, Q274K, Q355R, E419Q (all positions numbered according to EU numbering). In some embodiments, the Ab has a modified human IgG4 HC constant region comprising amino acid substitutions at the following amino acid residues: E137G, D203N, Q274K, Q355R, E419Q (all positions numbered according to EU numbering). In some embodiments, the Ab has a modified human IgG4 HC constant region comprising an amino acid substitution at any one or more of the following amino acid residues: Q274K, Q355R, E419Q (all positions numbered according to EU numbering). In some embodiments, the Ab has a modified human IgG4 HC constant region comprising an amino acid substitution at the following amino acid residues: Q274K, Q355R, E419Q (all positions numbered according to EU numbering).

In further embodiments, the Ab in conjugate of Formula I has a modified human IgG1 or human IgG4 constant domain comprising one or more engineered cysteine residues (see WO 2018/232088 A1). In such embodiments, the Ab comprises a cysteine at amino acid residue 124 (EU numbering) in heavy chain constant domain 1 (CH1), or a cysteine at amino acid residue 378 (EU numbering) in heavy chain constant domain 2 (CH2). In other embodiments, the Ab comprises a cysteine at amino acid residue 124 (EU numbering) in the CH1 domain and a cysteine at amino acid residue 378 (EU numbering) in the CH2 domain.

In some embodiment, the Ab in conjugate of Formula I is selected from Ab1, Ab2, Ab3, Ab4, Ab5, Ab6, Ab7, Ab8, Ab9 and Ab10. In some embodiments Ab1, Ab2, Ab3, Ab4, Ab5, Ab6, Ab7, and Ab8 have the same HCDR and LCDR amino acid sequences. As such, as shown below, Ab1, Ab2, Ab3, Ab4, Ab5, Ab6, Ab7, and Ab8, bind to an epitope comprising amino acid residues selected from D12, M14, S15, I16, L39, F41, L42, T48, C49, I50, E52, H62, L64, M65, D66, D67, V68, V69, D72, R99, P121, P123, P124, D125, P192 (the amino acid residue positions correspond to SEQ ID NO: 15). In some embodiments as shown below, Ab9 and Ab10 bind to an epitope of human IL-4Rα, wherein the epitope comprises one or more amino acid residues selected from D12, M14, S15, I16, Y37, L39, F41, L43, E45, H47, T48, C49, I50, H62, L64, M65, D66, D67, V69, D72, R99, P121, P123, P124, D125 (the amino acid residue positions correspond to SEQ ID NO: 15). In some embodiments, as shown below Ab1, Ab2, Ab3, Ab4, Ab5, Ab6, Ab7, Ab8, Ab9 and Ab10 bind to an epitope of human IL-4Rα, wherein the epitope comprises one or more amino acid residues selected from D12, M14, S15, I16, Y37, L39, T48, C49, I50, E52, H62, M65, R99, P121, P123, P124, D125, P192 (the amino acid residue positions correspond to SEQ ID NO: 15). In some embodiments, as shown below, Ab1, Ab2, Ab3, Ab4, Ab5, Ab6, Ab7, Ab8, Ab9 and Ab10 bind to an epitope of human IL-4Rα, wherein the epitope comprises one or more amino acid residue selected from R99, P121, P123, P124, D125, P192 (the amino acid residue positions correspond to SEQ ID NO: 15), wherein these residues are located in domain 2 of the N-terminal fibronectin type-III domains of the IL-4Rα. In some embodiments as shown below, Ab1, Ab2, Ab3, Ab4, Ab5, Ab6, Ab7, Ab8, Ab9 and Ab10 binds to an epitope of human IL-4Rα, wherein the epitope comprises one or more amino acid residue selected from D66, D67, and D125 (the amino acid residue positions correspond to SEQ ID NO: 15). In further embodiments as shown below, Ab1, Ab2, Ab3, Ab4, Ab5, Ab6, Ab7, Ab8, Ab9 and Ab10 binds to an epitope of human IL-4Rα, wherein the epitope comprises at least one of amino acid residues selected from D66 and D67 (the amino acid residue positions correspond to SEQ ID NO: 15). In yet further embodiments as shown below, Ab1, Ab2, Ab3, Ab4, Ab5, Ab6, Ab7, Ab8, Ab9 and Ab10 binds to an epitope of human IL-4Rα, wherein the epitope comprises at least one of amino acid residues selected from D66 and D125 (the amino acid residue positions correspond to SEQ ID NO: 15). In yet further embodiments as shown below, Ab1, Ab2, Ab3, Ab4, Ab5, Ab6, Ab7, Ab8, Ab9 and Ab10 binds to an epitope of human IL-4Rα, wherein the epitope comprises amino acid residue D66 (the amino acid residue positions correspond to SEQ ID NO: 15). In such embodiments, Ab1, Ab2, Ab3, Ab4, Ab5, Ab6, Ab7, Ab8, Ab9 and Ab10 bind a novel structural and/or functional epitope of the human IL-4Rα, wherein the epitope spans domain 1 and domain 2 of the n-terminal fibronectin type-III domains of the IL-4Rα. In particular embodiments, Ab1, Ab2, Ab3, Ab4, Ab5, Ab6, Ab7, Ab8, Ab9 and Ab10 bind a novel structural and/or functional epitope of the human IL-4Rα, wherein the epitope overlaps with the IL-4 binding site to IL-4Rα. In such embodiments, Ab1, Ab2, Ab3, Ab4, Ab5, Ab6, Ab7, Ab8, Ab9 and Ab10 inhibits binding of IL-4 to the human IL-4Rα. In particular embodiments, Ab1, Ab2, Ab3, Ab4, Ab5, Ab6, Ab7, Ab8, Ab9 and Ab10 bind a novel structural and/or functional epitope of the human IL-4Rα, wherein the epitope overlaps with the IL-13 binding sites to IL-4Rα. In such embodiments, Ab1, Ab2, Ab3, Ab4, Ab5, Ab6, Ab7, Ab8, Ab9 and Ab10 inhibits binding of IL-13 to the human IL-4Rα. In some embodiments, Ab1, Ab2, Ab3, Ab4, Ab5, Ab6, Ab7, Ab8, Ab9 and Ab10 bind a novel structural and/or functional epitope of the human IL-4Rα, wherein the epitope overlaps with the IL-4 and the IL-13 binding sites to IL-4Rα. In such embodiments, Ab1, Ab2, Ab3, Ab4, Ab5, Ab6, Ab7, Ab8, Ab9 and Ab10 inhibits binding of IL-4 and IL-13 to the human IL-4Rα. In some embodiments, the IL-4Rα epitope is determined by X-ray crystallography, alanine scanning mutagenesis, steric hindrance mutagenesis, and/or HDX-MS. In yet other embodiments, the IL-4Rα epitope is determined by site-directed mutagenesis.

In some embodiments, the present disclosure provides nucleic acids encoding a HC or LC, or a VH or VL, of an antibody that binds human IL-4Rα, or vectors comprising such nucleic acids.

In some embodiments, the present disclosure provides a nucleic acid comprising a sequence of SEQ ID NO: 11, 12, 14, 29, 30, 32, 34, 36, 38, 48, 49, 51, or 53.

In some embodiments, nucleic acids encoding a heavy chain or light chain of the antibodies that bind human IL-4Rα are provided. In some embodiments nucleic acids comprising a sequence encoding SEQ ID NO: 9, 10, 13, 27, 28, 31, 33, 35, 37, 46, 47, 50, or 52 are provided. In some embodiments, nucleic acids comprising a sequence encoding an antibody heavy chain that comprises SEQ ID NO: 9, 13, 27, 31, 33, 35, 37, 46, 50, or 52 are provided. For example, the nucleic acid can comprise a sequence of SEQ ID NO: 11, 14, 29, 32, 34, 36, 38, 48, 51, or 53. In some embodiments, nucleic acids comprising a sequence encoding an antibody light chain that comprises SEQ ID NO: 10, 28, or 47 are provided. For example, the nucleic acid can comprise a sequence of SEQ ID NO: 12, 30, or 49.

In some embodiments of the present disclosure, nucleic acids encoding a VH or VL of an antibody specifically binding human IL-4Rα are provided. In some embodiments, nucleic acids comprising a sequence encoding SEQ ID NO: 7, 8, 25, 26, 44, or 45 are provided. In some embodiments, nucleic acids comprising a sequence encoding an antibody VH that comprises SEQ ID NO: 7, 25, or 44 are provided. In some embodiments, nucleic acids comprising a sequence encoding an antibody VL that comprises SEQ ID NO: 8, 26, or 45 are provided.

Some embodiments of the present disclosure provide vectors comprising a nucleic acid sequence encoding an antibody heavy chain or light chain. For example, such vectors can comprise a nucleic acid sequence encoding SEQ ID NO: 9, 13, 27, 31, 33, 35, 37, 46, 50, or 52. In some embodiments, the vector comprises a nucleic acid sequence encoding SEQ ID NO: 10, 28, or 47.

Provided herein are also vectors comprising a nucleic acid sequence encoding an antibody VH or VL. For example, such vectors can comprise a nucleic acid sequence encoding SEQ ID NO: 7, 25, or 44. In some embodiments, the vector comprises a nucleic acid sequence encoding SEQ ID NO: 8, 26, or 45.

Provided herein are also vectors comprising a first nucleic acid sequence encoding an antibody heavy chain and a second nucleic acid sequence encoding an antibody light chain. In some embodiments, the vector comprises a first nucleic acid sequence encoding SEQ ID NO: 9, 13, 27, 31, 33, 35, 37, 46, 50, or 52 and a second nucleic acid sequence encoding SEQ ID NO: 10, 28, or 47. In some embodiments, the vector comprises a first nucleic acid sequence encoding SEQ ID NO: 9 and a second nucleic acid sequence encoding SEQ ID NO: 10. In some embodiments, the vector comprises a first nucleic acid sequence encoding SEQ ID NO: 13 and a second nucleic acid sequence encoding SEQ ID NO: 10. In some embodiments, the vector comprises a first nucleic acid sequence encoding SEQ ID NO: 27 and a second nucleic acid sequence encoding SEQ ID NO: 28. In some embodiments, the vector comprises a first nucleic acid sequence encoding SEQ ID NO: 31 and a second nucleic acid sequence encoding SEQ ID NO: 10. In some embodiments, the vector comprises a first nucleic acid sequence encoding SEQ ID NO: 33 and a second nucleic acid sequence encoding SEQ ID NO: 10. In some embodiments, the vector comprises a first nucleic acid sequence encoding SEQ ID NO: 35 and a second nucleic acid sequence encoding SEQ ID NO: 10. In some embodiments, the vector comprises a first nucleic acid sequence encoding SEQ ID NO: 37 and a second nucleic acid sequence encoding SEQ ID NO: 10. In some embodiments, the vector comprises a first nucleic acid sequence encoding SEQ ID NO: 50 and a second nucleic acid sequence encoding SEQ ID NO: 10. In some embodiments, the vector comprises a first nucleic acid sequence encoding SEQ ID NO: 52 and a second nucleic acid sequence encoding SEQ ID NO: 10. In some embodiments, the vector comprises a first nucleic acid sequence encoding SEQ ID NO: 46 and a second nucleic acid sequence encoding SEQ ID NO: 47.

Also provided herein are compositions comprising a first vector comprising a nucleic acid sequence encoding an antibody heavy chain, and a second vector comprising a nucleic acid sequence encoding an antibody light chain. In some embodiments, the composition comprises a first vector comprising a nucleic acid sequence encoding SEQ ID NO: 9, 13, 27, 31, 33, 35, 37, 46, 50, or 52 and a second vector comprising a nucleic acid sequence encoding SEQ ID NO: 10, 28, or 47. In some embodiments, the composition comprises a first vector comprising a nucleic acid sequence encoding SEQ ID NO: 9 and a second vector comprising a nucleic acid sequence encoding SEQ ID NO: 10. In some embodiments, the composition comprises a first vector comprising a nucleic acid sequence encoding SEQ ID NO: 13 and a second vector comprising a nucleic acid sequence encoding SEQ ID NO: 10. In some embodiments, the composition comprises a first vector comprising a nucleic acid sequence encoding SEQ ID NO: 27 and a second vector comprising a nucleic acid sequence encoding SEQ ID NO: 28. In some embodiments, the composition comprises a first vector comprising a nucleic acid sequence encoding SEQ ID NO: 31 and a second vector comprising a nucleic acid sequence encoding SEQ ID NO: 10. In some embodiments, the composition comprises a first vector comprising a nucleic acid sequence encoding SEQ ID NO: 33 and a second vector comprising a nucleic acid sequence encoding SEQ ID NO: 10. In some embodiments, the composition comprises a first vector comprising a nucleic acid sequence encoding SEQ ID NO: 35 and a second vector comprising a nucleic acid sequence encoding SEQ ID NO: 10. In some embodiments, the composition comprises a first vector comprising a nucleic acid sequence encoding SEQ ID NO: 37 and a second vector comprising a nucleic acid sequence encoding SEQ ID NO: 10. In some embodiments, the composition comprises a first vector comprising a nucleic acid sequence encoding SEQ ID NO: 50 and a second vector comprising a nucleic acid sequence encoding SEQ ID NO: 10. In some embodiments, the composition comprises a first vector comprising a nucleic acid sequence encoding SEQ ID NO: 52 and a second vector comprising a nucleic acid sequence encoding SEQ ID NO: 10. In some embodiments, the composition comprises a first vector comprising a nucleic acid sequence encoding SEQ ID NO: 46 and a second vector comprising a nucleic acid sequence encoding SEQ ID NO: 47.

Also provided herein are compositions comprising a vector comprising a nucleic acid sequence encoding an antibody heavy chain, and a nucleic acid sequence encoding an antibody light chain. In some embodiments, the composition comprises a vector comprising a nucleic acid sequence encoding SEQ ID NO: 9, 13, 27, 31, 33, 35, 37, 46, 50, or 52 and a second nucleic acid sequence encoding SEQ ID NO: 10, 28, or 47.

As used herein, “GC” in the Formula:

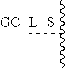

refers to a suitable glucocorticoid receptor agonist payload and includes the following Formulas IIa, IIb, or IIc:

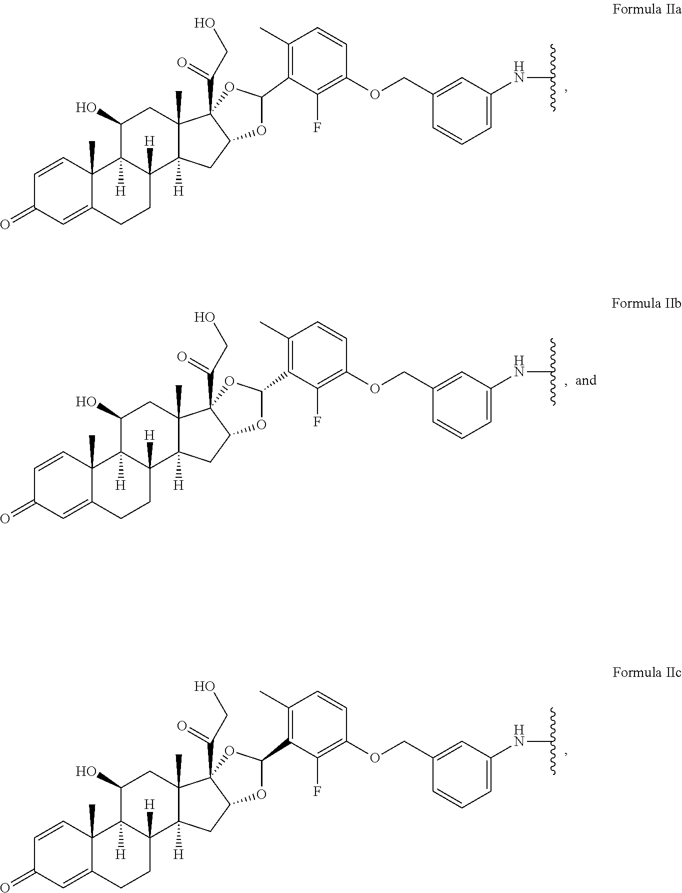

As used herein, “L” in the Formula

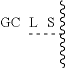

refers to a suitable linker group which connects Ab to the GC. Suitable linkers known to those of ordinary skill in the art include, for example, cleavable and noncleavable linkers. More specifically, suitable linkers “L” include the following of Formulas IIIa through IIIf:

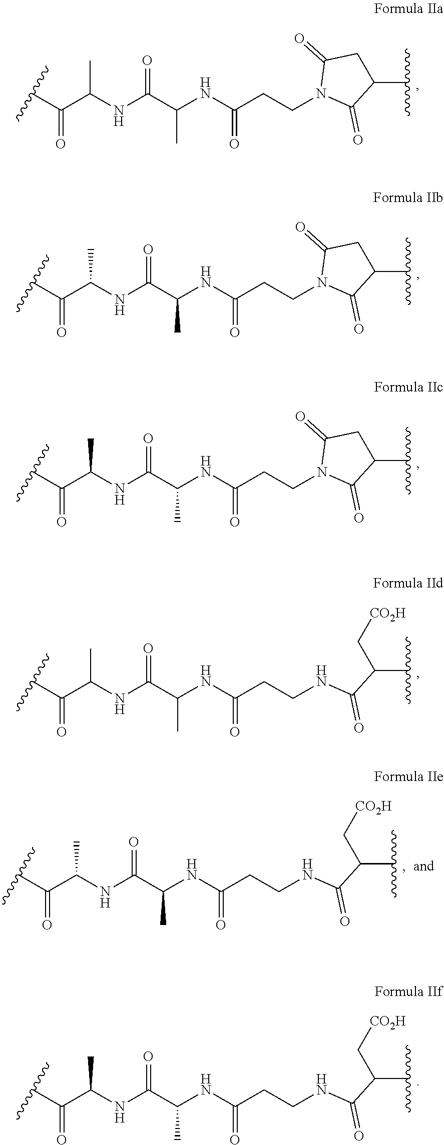 [SMILES: *C(=O)C(C)NC(=O)C(C)NC(=O)CCN1C(=O)CC(*)C1=O ; *C(=O)[C@H](C)NC(=O)[C@H](C)NC(=O)CCN1C(=O)CC(*)C1=O ; *C(=O)[C@@H](C)NC(=O)[C@@H](C)NC(=O)CCN1C(=O)CC(*)C1=O ; *C(=O)C(C)NC(=O)C(C)NC(=O)CCNC(=O)C(*)CC(=O)O ; *C(=O)[C@H](C)NC(=O)[C@H](C)NC(=O)CCNC(=O)C(*)CC(=O)O ; *C(=O)[C@@H](C)NC(=O)[C@@H](C)NC(=O)CCNC(=O)C(*)CC(=O)O]

In an embodiment, the disclosure provides a glucocorticoid receptor agonist payload-linker of Formula IV:

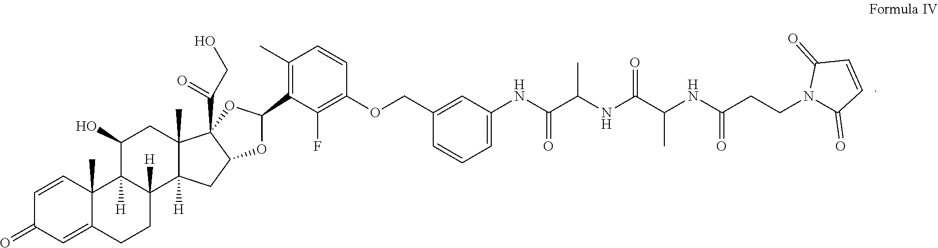

In an embodiment, the disclosure provides a glucocorticoid receptor agonist payload-linker of Formula IVa:

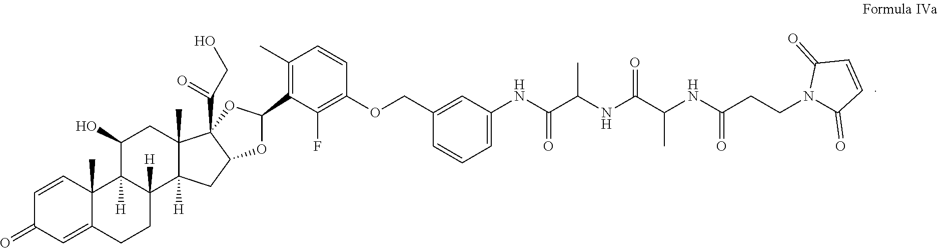

In an embodiment, the disclosure provides a glucocorticoid receptor agonist payload-linker of Formula IVb:

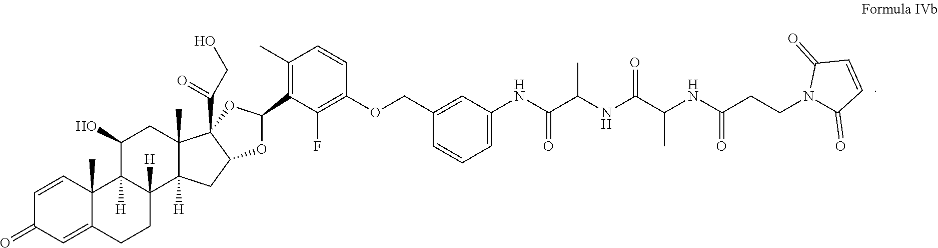

In an embodiment, the disclosure provides a glucocorticoid receptor agonist payload-linker of Formula IVc:

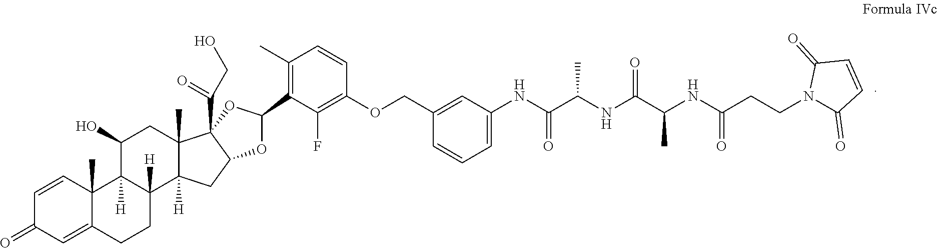

In an embodiment, the disclosure provides a glucocorticoid receptor agonist payload-linker of Formula IVd:

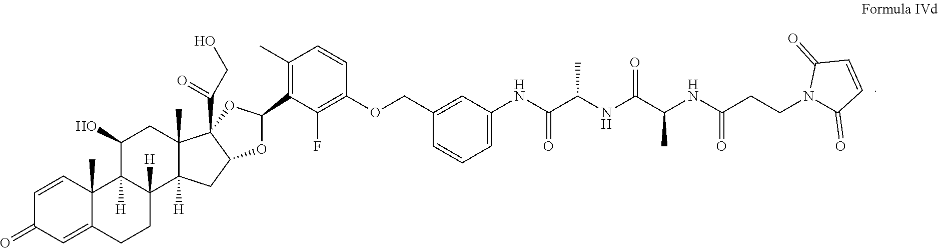

In an embodiment, the disclosure provides a compound of Formula V:

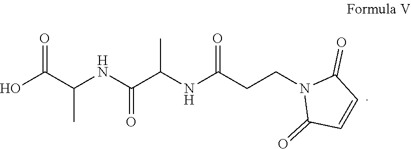 [SMILES: CC(NC(=O)C(C)NC(=O)CCN1C(=O)C=CC1=O)C(=O)O]

In a further embodiment, the disclosure provides a compound of Formula Va:

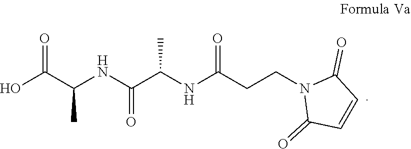 [SMILES: C[C@H](NC(=O)[C@H](C)NC(=O)CCN1C(=O)C=CC1=O)C(=O)O]

In an embodiment, the present disclosure also provides a method of treating an inflammatory disease in a subject in need thereof, comprising administering to the subject an effective amount of a conjugate of Formula I, or a pharmaceutically acceptable salt thereof. In an embodiment, the present disclosure also provides a method of treating an inflammatory disease, wherein the inflammatory disease is a Type 2 inflammatory disease in a subject in need thereof, comprising administering to the subject an effective amount of a conjugate of Formula I, or a pharmaceutical or salt thereof. In certain embodiments, the Type 2 inflammatory disease is for example, atopic dermatitis, eosinophilic esophagitis, nasal polyposis, asthma, chronic rhinosinusitis, allergic disease, chronic obstructive pulmonary disease, or chronic spontaneous urticaria. In an embodiment, the present disclosure further provides a method of treating atopic dermatitis in a subject in need thereof, comprising administering to the subject an effective amount of a conjugate of Formula I, or a pharmaceutically acceptable salt thereof. In an embodiment, the present disclosure further provides a method of treating eosinophilic esophagitis in a subject in need thereof, comprising administering to the subject an effective amount of a conjugate of Formula I, or a pharmaceutically acceptable salt thereof. In an embodiment, the present disclosure further provides a method of treating nasal polyposis in a subject in need thereof, comprising administering to the subject an effective amount of a conjugate of Formula I, or a pharmaceutically acceptable salt thereof. In an embodiment, the present disclosure further provides a method of treating asthma in a subject in need thereof, comprising administering to the subject an effective amount of a conjugate of Formula I, or a pharmaceutically acceptable salt thereof. In an embodiment, the present disclosure further provides a method of treating chronic rhinosinusitis in a subject in need thereof, comprising administering to the subject an effective amount of a conjugate of Formula I, or a pharmaceutically acceptable salt thereof. In an embodiment, the present disclosure further provides a method of treating allergic disease in a subject in need thereof, comprising administering to the subject an effective amount of a conjugate of Formula I, or a pharmaceutically acceptable salt thereof. In an embodiment, the present disclosure further provides a method of treating chronic obstructive pulmonary disease in a subject in need thereof, comprising administering to the subject an effective amount of a conjugate of Formula I, or a pharmaceutically acceptable salt thereof. In an embodiment, the present disclosure further provides a method of treating chronic spontaneous urticaria in a subject in need thereof, comprising administering to the subject an effective amount of a conjugate of Formula I, or a pharmaceutically acceptable salt thereof.

In an embodiment, the present disclosure further provides a conjugate of Formula I, or a pharmaceutically acceptable salt thereof for use in therapy. In an embodiment, the present disclosure provides a conjugate of Formula I, or a pharmaceutically acceptable salt thereof for use in the treatment of an inflammatory disease. In certain embodiments, the inflammatory disease is a Type 2 inflammatory disease. In certain embodiments, the Type 2 inflammatory disease is for example, atopic dermatitis, eosinophilic esophagitis, nasal polyposis, asthma, chronic rhinosinusitis, allergic disease, chronic obstructive pulmonary disease, or chronic spontaneous urticaria. In an embodiment, the present disclosure provides a conjugate of Formula I, or a pharmaceutically acceptable salt thereof, for use in the treatment of atopic dermatitis. In an embodiment, the present disclosure provides a conjugate of Formula I, or a pharmaceutically acceptable salt thereof, for use in the treatment of eosinophilic esophagitis. In an embodiment, the present disclosure provides a conjugate of Formula I, or a pharmaceutically acceptable salt thereof, for use in the treatment of nasal polyposis. In an embodiment, the present disclosure provides a conjugate of Formula I, or a pharmaceutically acceptable salt thereof, for use in the treatment of asthma. In an embodiment, the present disclosure provides a conjugate of Formula I, or a pharmaceutically acceptable salt thereof, for use in the treatment of chronic rhinosinusitis. In an embodiment, the present disclosure provides a conjugate of Formula I, or a pharmaceutically acceptable salt thereof, for use in the treatment of allergic disease. In an embodiment, the present disclosure provides a conjugate of Formula I, or a pharmaceutically acceptable salt thereof, for use in the treatment of chronic obstructive pulmonary disease. In an embodiment, the present disclosure provides a conjugate of Formula I, or a pharmaceutically acceptable salt thereof, for use in the treatment of chronic spontaneous urticaria.

In an embodiment, the present disclosure also provides the use of a conjugate of Formula I, or a pharmaceutically acceptable salt thereof, in the manufacture of a medicament for the treatment of an inflammatory disease. In certain embodiments, the inflammatory disease is a Type 2 inflammatory disease. In certain embodiments, the Type 2 inflammatory disease is for example, atopic dermatitis, eosinophilic esophagitis, nasal polyposis, asthma, chronic rhinosinusitis, allergic disease, chronic obstructive pulmonary disease, or chronic spontaneous urticaria. In an embodiment, the present disclosure provides the use of a conjugate of Formula I, or a pharmaceutically acceptable salt thereof, in the manufacture of a medicament for the treatment of atopic dermatitis. In an embodiment, the present disclosure provides the use of a conjugate of Formula I, or a pharmaceutically acceptable salt thereof, in the manufacture of a medicament for the treatment of eosinophilic esophagitis. In an embodiment, the present disclosure provides the use of a conjugate of Formula I, or a pharmaceutically acceptable salt thereof, in the manufacture of a medicament for the treatment of nasal polyposis. In an embodiment, the present disclosure provides the use of a conjugate of Formula I, or a pharmaceutically acceptable salt thereof, in the manufacture of a medicament for the treatment of asthma. In an embodiment, the present disclosure provides the use of a conjugate of Formula I, or a pharmaceutically acceptable salt thereof, in the manufacture of a medicament for the treatment of chronic rhinosinusitis. In an embodiment, the present disclosure provides the use of a conjugate of Formula I, or a pharmaceutically acceptable salt thereof, in the manufacture of a medicament for the treatment of allergic disease. In an embodiment, the present disclosure provides the use of a conjugate of Formula I, or a pharmaceutically acceptable salt thereof, in the manufacture of a medicament for the treatment of chronic obstructive pulmonary disease. In an embodiment, the present disclosure provides the use of a conjugate of Formula I, or a pharmaceutically acceptable salt thereof, in the manufacture of a medicament for the treatment of chronic spontaneous urticaria.

Nucleic acids of the present disclosure may be expressed in a host cell, for example, after the nucleic acids have been operably linked to an expression control sequence. Expression control sequences capable of expression of nucleic acids to which they are operably linked are well known in the art. An expression vector may include a sequence that encodes one or more signal peptides that facilitate secretion of the polypeptide(s) from a host cell. Expression vectors containing a nucleic acid of interest (e.g., a nucleic acid encoding a heavy chain or light chain of an antibody) may be transferred into a host cell by well-known methods, e.g., stable or transient transfection, transformation, transduction or infection. Additionally, expression vectors may contain one or more selection markers, e.g., tetracycline, neomycin, and dihydrofolate reductase, to aide in detection of host cells transformed with the desired nucleic acid sequences.

In another aspect, provided herein are cells, e.g., host cells, comprising the nucleic acids, vectors, or nucleic acid compositions described herein. A host cell may be a cell stably or transiently transfected, transformed, transduced or infected with one or more expression vectors expressing all or a portion of an antibody described herein. In some embodiments, a host cell may be stably or transiently transfected, transformed, transduced or infected with an expression vector expressing HC and LC polypeptides of an antibody of the present disclosure. In some embodiments, a host cell may be stably or transiently transfected, transformed, transduced, or infected with a first vector expressing HC polypeptides and a second vector expressing LC polypeptides of an antibody described herein. Such host cells, e.g., mammalian host cells, can express the antibodies that bind human IL-4Rα as described herein. Mammalian host cells known to be capable of expressing antibodies include CHO cells, HEK293 cells, COS cells, and NS0 cells.

In some embodiments, the cell, e.g., host cell, comprises a vector comprising a first nucleic acid sequence encoding SEQ ID NO: 9, 13, 27, 31, 33, 35, 37, 46, 50, or 52 and a second nucleic acid sequence encoding SEQ ID NO: 10, 28, or 47.

In some embodiments, the cell, e.g., host cell, comprises a first vector comprising a nucleic acid sequence encoding SEQ ID NO: 9, 13, 27, 31, 33, 35, 37, 46, 50, or 52 and a second vector comprising a nucleic acid sequence encoding SEQ ID NO: 10, 28, or 47.

The present disclosure further provides a process for producing an antibody that binds human IL-4Rα described herein by culturing the host cell described above, e.g., a mammalian host cell, under conditions such that the antibody is expressed and recovering the expressed antibody from the culture medium. The culture medium, into which an antibody has been secreted, may be purified by conventional techniques. Various methods of protein purification may be employed, and such methods are known in the art and described, for example, in Deutscher, Methods in Enzymology 182: 83-89 (1990) and Scopes, Protein Purification: Principles and Practice, 3^{rd} Edition, Springer, NY (1994).

The present disclosure provides a method of producing a conjugate, the method comprising conjugating a compound of the present disclosure with an anti-human IL-4Rα antibody.

The present disclosure provides a method of producing a conjugate, the method comprising conjugating the compound of Formula IV with an anti-human IL-4Rα antibody. The present disclosure provides a method of producing a conjugate, the method comprising conjugating the compound of Formula IVa with an anti-human IL-4Rα antibody. The present disclosure provides a method of producing a conjugate, the method comprising conjugating the compound Formula IVb with an anti-human IL-4Rα antibody. The present disclosure provides a method of producing a conjugate, the method comprising conjugating the compound Formula IVc with an anti-human IL-4Rα antibody. The present disclosure provides a method of producing a conjugate, the method comprising conjugating the compound Formula IVd with an anti-human IL-4Rα antibody.

In some embodiments, the conjugate being produced is the conjugate of Formula I.

The present disclosure provides a method of producing a conjugate, the method comprising the steps of:
(a) reducing an anti-human IL-4Rα antibody with a reducing agent to produce a reduced anti-human IL-4R antibody, wherein the anti-human IL-4Rα antibody comprises one or more engineered cysteine residues;
(b) oxidizing the reduced anti-human IL-4Rα antibody with an oxidizing agent to produce an oxidized anti-human IL-4R antibody; and
(c) contacting the oxidized anti-human IL-4Rα antibody with a compound of the present disclosure to produce the conjugate.

The present disclosure provides a method of producing a conjugate, the method comprising the steps of:
(a) reducing an anti-human IL-4Rα antibody with a reducing agent, wherein the anti-human IL-4Rα antibody comprises one or more engineered cysteine residue;
(b) oxidizing the anti-human IL-4Rα antibody with an oxidizing agent to produce an oxidized anti-human IL-4R antibody; and
(c) contacting the oxidized anti-human IL-4Rα antibody with a compound of the formula

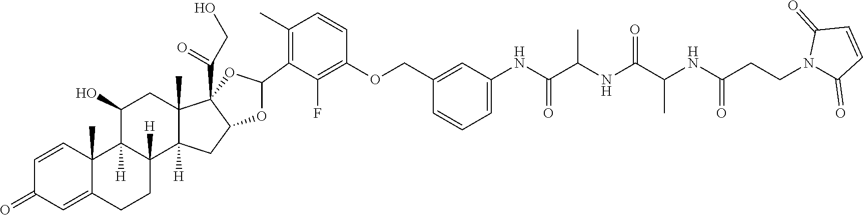

to produce the conjugate.

In some embodiments, the reducing agent is dithiothreitol. In some embodiments, the oxidizing agent is dehydroascorbic acid. In some embodiments, the reducing agent is dithiothreitol and the oxidizing agent is dehydroascorbic acid.

The present disclosure further provides antibodies or antigen binding fragments thereof produced by any of the processes described herein.

In an embodiment, the present disclosure further provides a pharmaceutical composition, comprising a conjugate of Formula I, or a pharmaceutically acceptable salt thereof, or an antibody, nucleic acid, or vector described herein with one or more pharmaceutically acceptable carriers, diluents, or excipients. In an embodiment, the present disclosure further provides a pharmaceutical composition, comprising a conjugate of Formula I, or a pharmaceutically acceptable salt thereof, with one or more pharmaceutically acceptable carriers, diluents, or excipients. In an embodiment, the present disclosure further provides a pharmaceutical composition, comprising a conjugate of Formula I with one or more pharmaceutically acceptable carriers, diluents, or excipients. In an embodiment, the present disclosure further provides a process for preparing a pharmaceutical composition, comprising admixing a conjugate of Formula I, or a pharmaceutically acceptable salt thereof, with one or more pharmaceutically acceptable carriers, diluents, or excipients. In an embodiment, the present disclosure also encompasses novel intermediates and processes for the synthesis of conjugates of Formula I.

### DETAILED DESCRIPTION OF THE INVENTION

The term “IL-4Rα” as used herein, unless stated otherwise, refers to any native, mature IL-4Rα that results from processing of an IL-4Rα precursor protein in a cell. The term includes IL-4Rα from any vertebrate source, including mammals such as primates (e.g., humans and cynomolgus or rhesus monkeys), unless otherwise indicated. The term also includes naturally occurring variants of IL-4Rα, e.g., splice variants or allelic variants. The amino acid sequence of an example of human IL-4Rα is known in the art, e.g., UniProt reference sequence P24394 (SEQ ID NO: 39). The amino acid sequence of an example of cynomolgus monkey IL-4Rα is also known in the art, e.g., NCBI reference sequence XP_005591572.2 (SEQ ID NO: 40). The term “human IL-4Rα” is used herein to refer collectively to all known human IL-4Rα isoforms and polymorphic forms. Sequence numbering used herein is based on the mature protein without the signal peptide.

The term “IL-4R” as used herein, unless stated otherwise, refers to a complex of the IL-4Rα subunit with a common γ chain (Type I receptor) and/or a complex of the IL-4Rα subunit with an IL-13Rα1 (Type II receptor).

The term “IL-4” as used herein, unless stated otherwise, refers to any native, mature IL-4 that results from processing of an IL-4 precursor protein in a cell. The term includes IL-4 from any vertebrate source, including mammals such as primates (e.g., humans and cynomolgus or rhesus monkeys), unless otherwise indicated. The term also includes naturally occurring variants of IL-4, e.g., splice variants or allelic variants. The amino acid sequence of an example of human IL-4 is known in the art, e.g., UniProt reference sequence P05112 (SEQ ID NO: 17). The term “human IL-4” is used herein to refer collectively to all known human IL-4 isoforms and polymorphic forms.

The term “IL-13” as used herein, unless stated otherwise, refers to any native, mature IL-13 that results from processing of an IL-13 precursor protein in a cell. The term includes IL-13 from any vertebrate source, including mammals such as primates (e.g., humans and cynomolgus or rhesus monkeys), unless otherwise indicated. The term also includes naturally occurring variants of IL-13, e.g., splice variants or allelic variants. The amino acid sequence of an example of human IL-13 is known in the art, e.g., UniProt reference sequence P35225 (SEQ ID NO: 18). The term “human IL-13” is used herein to refer collectively to all known human IL-13 isoforms and polymorphic forms.

The term “CD23” as used herein, unless stated otherwise, refers to any native, mature CD23 that results from processing of a CD23 precursor protein in a cell. The term includes CD23 from any vertebrate source, including mammals such as primates (e.g., humans and cynomolgus or rhesus monkeys), unless otherwise indicated. The term also includes naturally occurring variants of CD23, e.g., splice variants or allelic variants. The amino acid sequence of an example of human CD23 is known in the art, e.g., UniProt reference sequence P06734 (SEQ ID NO: 41). The term “human CD23” is used herein to refer collectively to all known human CD23 isoforms and polymorphic forms.

The term “IL-4R associated disorder” as used herein refers to a disorder associated with IL-4R mediated signaling, such as for example disorders associated with IL-4R Type 1 and IL-4R Type II signaling. Such an IL-4R associated disorder may for example include immune inflammatory disorders. Such immune inflammatory disorders may include Type 2 inflammatory disorders, as disclosed herein. IL-4R associated disorder may further include cancer.

The term, “modulate” or “modulates” and the like, as used herein, refers to altering or changing a measurable value and includes both altering or changing such a measurable value upwards (i.e., upmodulate or upmodulating) or downwards (i.e., downmodulate or downmodulating).

The term “antibody” as used herein, refers to an immunoglobulin molecule that binds an antigen. Embodiments of an antibody include a monoclonal antibody, polyclonal antibody, human antibody, humanized antibody, chimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4). Embodiments of the present disclosure also include antibody fragments or antigen binding fragments, the term “antibody fragments or antigen binding fragments” comprise at least a portion of an antibody retaining the ability to interact with an antigen such as for example, Fab, Fab′, F(ab′)2, Fv fragments, scFv, scFab, disulfide-linked Fvs (sdFv), a Fd fragment or linear antibodies, which may be for example, fused to an Fc region or an IgG heavy chain constant region.

An exemplary antibody is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. The heavy chain constant region refers to a region of an antibody, which comprises the Fc region and CH1 domain of the antibody heavy chain. Each light chain is comprised of a light chain variable region (VL) and a light chain constant region. The IgG isotype may be further divided into subclasses (e.g., IgG1, IgG2, IgG3, and IgG4). The numbering of the amino acid residues in the constant region is based on the EU index as in Kabat. Kabat et al, Sequences of Proteins of Immunological Interest, 5^{th} edition, Bethesda, MD: U.S. Dept. of Health and Human Services, Public Health Service, National Institutes of Health (1991). The term EU Index numbering or EU numbering is used interchangeably herein.

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212). A combination of IMGT and North CDR definitions were used for the exemplified anti-human IL-4Rα antibodies as described herein.

The term “Fc region” as used herein, refers to a region of an antibody, which comprises the CH2 and CH3 domains of the antibody heavy chain. Optionally, the Fc region may include a portion of the hinge region or the entire hinge region of the antibody heavy chain. Biological activities such as effector function are attributable to the Fc region, which vary with the antibody isotype. Examples of antibody effector functions include, Fc receptor binding, antibody-dependent cell mediated cytotoxicity (ADCC), antibody-dependent cell mediated phagocytosis (ADCP), C1q binding, complement dependent cytotoxicity (CDC), phagocytosis, down regulation of cell surface receptors (e.g. B cell receptor) and B cell activation.

The term “epitope” as used herein, refers to the amino acid residues of an antigen, that are bound by an antibody. An epitope can be a linear epitope, a conformational epitope, or a hybrid epitope. The term “epitope” may be used in reference to a structural epitope. A structural epitope, according to some embodiments, may be used to describe the region of an antigen which is covered by an antibody (e.g., an antibody's footprint when bound to the antigen). In some embodiments, a structural epitope may describe the amino acid residues of the antigen that are within a specified proximity (e.g., within a specified number of Angstroms) of an amino acid residue of the antibody. The term “epitope” may also be used in reference to a functional epitope. A functional epitope, according to some embodiments, may be used to describe amino acid residues of the antigen that interact with amino acid residues of the antibody in a manner contributing to the binding energy between the antigen and the antibody. An epitope can be determined according to different experimental techniques, also called “epitope mapping techniques.” It is understood that the determination of an epitope may vary based on the different epitope mapping techniques used and may also vary with the different experimental conditions used, e.g., due to the conformational changes or cleavages of the antigen induced by specific experimental conditions. Epitope mapping techniques are known in the art (e.g., Rockberg and Nilvebrant, Epitope Mapping Protocols: Methods in Molecular Biology, Humana Press, 3^{rd} ed. 2018; Holst et al., Molecular Pharmacology 1998, 53(1): 166-175), including but not limited to, X-ray crystallography, nuclear magnetic resonance (NMR) spectroscopy, site-directed mutagenesis, species swap mutagenesis, alanine-scanning mutagenesis, steric hindrance mutagenesis, hydrogen-deuterium exchange (HDX), and cross-blocking assays.

The terms “bind” and “binds” as used herein, are intended to mean, unless indicated otherwise, the ability of a protein or molecule to form a chemical bond or attractive interaction with another protein or molecule, which results in proximity of the two proteins or molecules as determined by common methods known in the art.

The term “nucleic acid” as used herein, refer to polymers of nucleotides, including single-stranded and/or double-stranded nucleotide-containing molecules, such as DNA, cDNA and RNA molecules, incorporating native, modified, and/or analogs of, nucleotides. Polynucleotides of the present disclosure may also include substrates incorporated therein, for example, by DNA or RNA polymerase or a synthetic reaction.

Embodiments of the present disclosure include conjugates where a polypeptide (e.g., anti-human interleukin-4 receptor alpha antibody) is conjugated to one or more drug moieties, such as 2 drug moieties, 3 drug moieties, 4 drug moieties, 5 drug moieties, or more drug moieties. The drug moieties may be conjugated to the polypeptide at one or more sites in the polypeptide, as described herein. In certain embodiments, the conjugates have an average drug-to-antibody ratio (DAR) (molar ratio) in the range of from 2 to 5, or from 3 to 5, or from 3 to 4. In certain embodiments, the conjugates have an average DAR from 3 to 4. In certain embodiments, the conjugates have an average DAR of about 3. In certain embodiments, the conjugates have an average DAR of about 4.

As used herein, it is understood that the conjugate of Formula I encompasses conjugates of Formulas Ia, Ib, Ic, Id, Ie, and If, and all references to the conjugate of Formula I herein should be read as including conjugates of Formulas Ia, Ib, Ic, Id, Ie, and If. It is further understood by one of skill in the art that the conjugate of Formula I including conjugates of Formulas Ia, Ib, Ic, Id, Ie, and If can also be referred to as anti-human IL-4Rα antibody glucocorticoid conjugates (“anti-human IL-4Rα Ab GC conjugates”).

The anti-human IL-4Rα antibody GC conjugates of the present disclosure can be formulated as pharmaceutical compositions administered by any route which makes the conjugate bioavailable including, for example, intravenous or subcutaneous administration. Such pharmaceutical compositions can be prepared using techniques and methods known in the art (See, e.g., Remington: The Science and Practice of Pharmacy, A. Adejare, Editor, 23n d Edition, published 2020, Elsevier Science).

As used herein, the terms “treating”, “treatment”, or “to treat” includes restraining, slowing, stopping, controlling, delaying, or reversing the progression or severity of an existing symptom or disorder, or ameliorating the existing symptom or disorder, but does not necessarily indicate a total elimination of the existing symptom or disorder. Treatment includes administration of a protein or nucleic acid or vector or composition for treatment of a symptom or disorder in a patient, particularly in a human.

The term “inhibits” or “inhibiting” as used herein, refers to for example, a reduction, lowering, slowing, decreasing, stopping, disrupting, abrogating, antagonizing, or blocking of a biological response or activity, but does not necessarily indicate a total elimination of a biological response or activity.

As used herein, the term “subject” refers to a mammal, including, but are not limited to, a human, chimpanzee, ape, monkey, cattle, horse, sheep, goat, swine, rabbit, dog, cat, rat, mouse, guinea pig, and the like. Preferably the subject is a human.

As used herein, the term “effective amount” refers to the amount or dose of conjugate of the disclosure, or a pharmaceutically acceptable salt thereof which, upon single or multiple dose administration to the subject, provides the desired effect in the subject under diagnosis or treatment. The term “effective amount”, as used herein, further refers to an amount or dose of conjugates of the disclosure, or a pharmaceutically acceptable salt thereof, that will elicit the desired biological or medical response of a subject, for example, reduction or inhibition of a protein activity, or ameliorate symptoms, alleviate conditions, slow or delay disease progression, or prevent a disease, etc. In a non-limiting embodiment, the term “effective amount” refers to the amount necessary (at dosages and for periods of time and for the means of administration) of or dose of conjugate of the disclosure, or a pharmaceutically acceptable salt thereof, when administered to a subject, is effective to at least partially alleviate, inhibit, prevent and/or ameliorate a condition, or a disorder or a disease to achieve the desired therapeutic result. An effective amount is also one in which any toxic or detrimental effects of or dose of conjugate of the disclosure, or a pharmaceutically acceptable salt thereof of the present disclosure are outweighed by the beneficial effects.

An effective amount can be determined by one skilled in the art by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular conjugate administered; the mode of administration; the bioavailability characteristics of the preparation administered; the dose regimen selected; the use of concomitant medication; and other relevant circumstances.

Included within the scope of the present invention is a pharmaceutically acceptable salt of the conjugate of Formula I. A pharmaceutically acceptable salt of a conjugate of the invention, such as a conjugate of Formula I can be formed under standard conditions known in the art. See, for example, Berge, S. M., et al., “Pharmaceutical Salts,” Journal of Pharmaceutical Sciences, 66: 1-19, (1977).

TABLE 1
Abbreviations and definitions

| Term | Definition |
|---|---|
| ACN | acetonitrile |
| aq | aqueous |
| C18 | octadecylsilane |
| cP | centipoise |
| DCM | dichloromethane |
| DIPEA | N,N-diisopropylethylamine |
| DMF | N,N-dimethylformamide |
| DMSO | dimethyl sulfoxide |
| dppf | 1,1′-bis(diphenylphosphino)ferrocene |
| ES/MS | electrospray mass spectrometry |
| EtOAc | ethyl acetate |
| HATU | hexafluorophosphate azabenzotriazole tetramethyl uronium |
| HIC | hydrophobic interaction chromatography |
| HPLC | high performance liquid chromatography |
| LDA | lithium diisopropylamide |
| MeOH | methanol |
| MS | mass spectrometry |
| MTBE | methyl tert-butyl ether |
| m/z | mass-to-charge ratio |
| NMR | nuclear magnetic resonance |
| Pet ether | petroleum ether |
| RP-HPLC | reverse-phase HPLC |
| rt | room temperature |
| satd | saturated |
| THF | tetrahydrofuran |

The conjugates of the present disclosure, or salts thereof, may be readily prepared by a variety of procedures known to one of ordinary skill in the art, some of which are illustrated in the preparations and examples below. One of ordinary skill in the art recognizes that the specific synthetic steps for each of the routes described may be combined in different ways, or in conjunction with steps from different schemes, to prepare conjugates of the disclosure, or salts thereof. The product of each step can be recovered by conventional methods well known in the art, including extraction, evaporation, precipitation, chromatography, filtration, trituration, and crystallization. All substituents unless otherwise indicated, are as previously defined. The reagents and starting materials are readily available to one of ordinary skill in the art. The following preparations, examples, and assays further illustrate the invention, but should not be construed to limit the scope of the invention in any way.

### BRIEF DESCRIPTION OF THE DRAWINGS

FIG. 1 shows X-ray crystal structure overlay of a Fab portion of Ab9 bound to IL-4Rα ECD with the crystal structure of a dupilumab Fab portion with Crystal Kappa design complexed with human IL-4Rα (pdb accession code 6WGL).

FIG. 2 shows the functional epitope amino acid residue locations (human IL-4Rα residues Asp66 and Asp125) in the crystal structure of Ab10 Fab portion with Crystal Kappa design complexed with human IL-4Rα ECD.

FIG. 3 shows X-ray crystal structure overlay of a Fab portion of Ab1 bound to IL-4Rα ECD with the crystal structure of a dupilumab Fab portion with Crystal Kappa design complexed with human IL-4Rα (pdb accession code 6WGL).

FIG. 4 shows the human IL-4Rα amino acid residue locations Asp66, Asp67, and Asp125 (all identified in the structural epitope; additionally, Asp66 identified in functional epitope) in the crystal structure of Ab1 Fab portion with Crystal Kappa design complexed with human IL-4Rα ECD.

FIG. 5 shows the inhibition of anti-CD40 induced B cell proliferation by the Ab1 GC conjugate of Example 1b.

FIGS. 6A-6B show the inhibition of IL-4 induced CD23 expression (6A) and GC-induced CD163 expression (6B) in myeloid cells by the Ab1 GC conjugate of Example 1b.

FIGS. 7A-7C show that the Ab1 GC conjugate of Example 1b significantly inhibited IL-4Rα mediated MDC (7A), GM-CSF (7B), and glucocorticoid receptor mediated IL-5 (7C) cytokine secretion.

FIG. 8 shows that the Ab1 GC conjugate of Example 1b does not significantly induce ADCC activity.

FIG. 9 shows that the Ab1 GC conjugate of Example 1b does not induce CDC activity in Daudi cells.

FIGS. 10A-10C show the differential scanning calorimetry (DSC) thermograms for the exemplified Ab1 GC conjugate of Example 1b in PBS, pH7.2 (10A); Acetate, pH5 (10B); and Histidine, pH6 (10C).

### PREPARATION 1

### 6-Bromo-2-fluoro-3-methoxybenzaldehyde

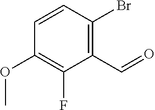

Two reactions were carried out in parallel. To a solution of 4-bromo-2-fluoro-1-methoxybenzene (250 g, 1.2 mol) in THF (1500 mL) was added LDA (2 M, 730 mL) slowly at −78° C., over 30 min. After an additional 30 min, DMF (140 mL, 1.8 mol) was added at −78° C. slowly over 30 min. After 1 h, the two reactions were combined and the mixture was diluted with aq citric acid (2000 mL) and extracted with EtOAc (1500 mL×2). The combined organic layers were washed with satd aq NaCl (1000 mL) and concentrated under reduced pressure to give a residue. The residue was triturated with petroleum ether (1000 mL) at rt over 12 h to give the title compound (382 g, 67% yield). ES/MS m/z 233.9 (M+H).

### PREPARATION 2

### 2-Fluoro-3-methoxy-6-methylbenzaldehyde

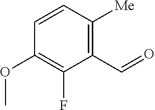

Three reactions were carried out in parallel. 6-Bromo-2-fluoro-3-methoxybenzaldehyde (120 g, 5.3 mol), methylboronic acid (47 g, 7.9 mol), Pd(dppf)Cl₂ (12 g, 0.02 mol), and Cs₂CO₃ (340 g, 1.1 mol) were added to a mixture of 1,4-dioxane (600 mL) and water (120 mL). The mixture was stirred at 120° C. After 12 h, the three reactions were combined and the mixture was diluted with satd aq NH₄Cl (1000 mL) and extracted with MTBE (1500 mL×2). The combined organic layers were washed with satd aq NaCl (1000 mL) and concentrated under reduced pressure to give a residue. The residue was purified by silica gel chromatography, eluting with 40:1 Pet ether:EtOAc to give the title compound (180 g, 59%). ES/MS m/z 169.3 (M+H).

### PREPARATION 3

### 2-Fluoro-3-hydroxy-6-methylbenzaldehyde

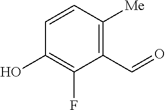 [SMILES: Cc1ccc(O)c(F)c1C=O]

2-Fluoro-3-methoxy-6-methylbenzaldehyde (175 g, 1.0 mol) was added into DCM (1050 mL). BBr₃ (200 mL, 2.1 mol) was added slowly into the solution at 0° C. The reaction was stirred at rt. After 1 h, the mixture was diluted with satd aq NaHCO₃ (1000 mL) until pH=7-8 and then extracted with MTBE (1500 mL×2). The combined organic layers were washed with satd aq NaCl (1000 mL) and concentrated under reduced pressure to give the title compound (110 g, 68%). ES/MS m/z 154.9 (M+H).

### PREPARATION 4

### tert-Butyl N-[3-[(2-fluoro-3-formyl-4-methyl-phenoxy)methyl]phenyl]carbamate

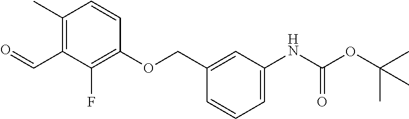 [SMILES: Cc1ccc(OCc2cccc(NC(=O)OC(C)(C)C)c2)c(F)c1C=O]

2-Fluoro-3-hydroxy-6-methylbenzaldehyde (130 g, 0.84 mol), tert-butyl (3-(bromomethyl)phenyl)carbamate (200 g, 0.70 mol), and potassium carbonate (350 g, 2.5 mol) were added in acetonitrile (780 mL) at rt and then heated to 50° C. After 5 h, the reaction was diluted with water (600 mL) and extracted with EtOAc (800 mL×2). The combined organic layers were washed with satd aq NaCl (800 mL) and concentrated under reduced pressure to give a residue. The residue was purified by silica gel chromatography, eluting with 50:1 Pet ether:EtOAc to give the crude product. The crude product was triturated with MTBE (500 mL) at rt for 30 min to give the title compound (103 g, 32%). ES/MS m/z 382.1 (M+Na).

### PREPARATION 5

### (6aR,6b S,7S,8aS,8b S,10R,11aR,12aS,12b S)-10-(3-((3-Aminobenzyl)oxy)-2-fluoro-6-methylphenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

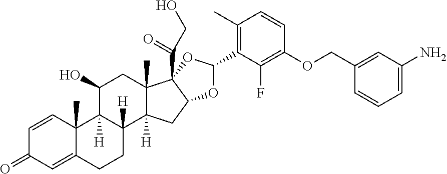

Perchloric acid (70% in water, 4.8 mL) was added to a suspension of (8S,9S,10R,11S,13S,14S,16R,17S)-11,16,17-trihydroxy-17-(2-hydroxyacetyl)-10,13-dimethyl-7,8,9,11,12,14,15,16-octahydro-6H-cyclopenta[a]phenanthren-3-one (4.4 g, 12 mmol, also referred to as “16alpha-hydroxyprednisolone”) and tert-butyl N-[3-[(2-fluoro-3-formyl-4-methyl-phenoxy)methyl]phenyl]carbamate (4.0 g, 11 mmol, preparation 4) in acetonitrile (110 mL) at −10° C. and was warmed to rt. After 1 h, DMF (10 mL) was added to the suspension at rt. After 18 h, the reaction was quenched with satd aq NaHCO₃ and extracted with 9:1 DCM:isopropanol. The organic layers were combined, dried over MgSO₄, filtered, and concentrated under reduced pressure to give a residue. The residue was purified by reverse phase chromatography, eluting with 1:1 aq NH₄HCO₃ (10 mM+5% MeOH):ACN to give the title compound, peak 1 (1.72 g, 25%). ES/MS m/z 618.6 (M+H). ¹H NMR (400.13 MHz, d₆-DMSO) δ 0.93-0.87 (m, 6H), 1.40 (s, 3H), 1.71-1.60 (m, 1H), 1.89-1.76 (m, 4H), 2.18-2.12 (m, 2H), 2.29 (s, 4H), 4.23-4.17 (m, 1H), 4.32-4.30 (m, 1H), 4.50-4.43 (m, 1H), 4.81 (d, J=3.2 Hz, 1H), 4.98-4.95 (m, 3H), 5.16-5.10 (m, 3H), 5.61 (s, 1H), 5.95 (s, 1H), 6.18-6.15 (m, 1H), 6.53-6.48 (m, 2H), 6.58 (s, 1H), 6.90-6.86 (m, 1H), 6.99 (t, J=7.7 Hz, 1H), 7.12 (t, J=8.5 Hz, 1H), 7.33-7.30 (m, 1H).

### PREPARATION 6

### (6aR,6b S,7S,8aS,8b S,10S,11aR,12aS,12b S)-10-(3-((3-Aminobenzyl)oxy)-2-fluoro-6-methylphenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (Herein Also Referred to as GC1)

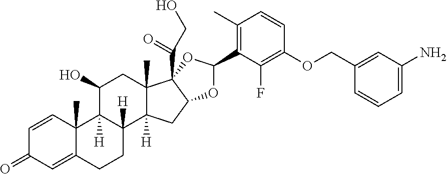

From Preparation 5, the residue was purified by reverse phase chromatography, eluting with 1:1 aq NH₄HCO₃ (10 mM+5% MeOH):ACN to give the title compound, peak 2 (1.24 g, 18%). ES/MS m/z 618.6 (M+H). 1H NMR (400.13 MHz, d₆-DMSO) d ¹H NMR (400.13 MHz, DMSO): 0.88 (s, 3H), 1.24-1.12 (m, 2H), 1.40 (s, 3H), 1.69-1.56 (m, 1H), 1.91-1.76 (m, 4H), 2.08-2.01 (m, 2H), 2.22 (s, 3H), 2.39-2.29 (m, 1H), 3.18 (d, J=5.2 Hz, 1H), 4.12-4.00 (m, 1H), 4.37-4.30 (m, 2H), 4.79 (d, J=3.1 Hz, 1H), 5.00-4.93 (m, 2H), 5.10-5.06 (m, 3H), 5.31 (d, J=6.7 Hz, 1H), 5.95 (s, 1H), 6.18 (dd, J=1.8, 10.1 Hz, 1H), 6.34 (s, 1H), 6.53-6.48 (m, 2H), 6.58 (s, 1H), 6.87 (d, J=8.5 Hz, 1H), 6.99 (t, J=7.7 Hz, 1H), 7.09 (t, J=8.5 Hz, 1H), 7.33 (d, J=10.1 Hz, 1H).

### PREPARATION 7

### (3-(2,5-Dioxo-2,5-dihydro-1H-pyrrol-1-yl)propanoyl)-L-alanyl-L-alanine

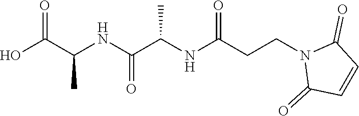 [SMILES: C[C@H](NC(=O)[C@H](C)NC(=O)CCN1C(=O)C=CC1=O)C(=O)O]

To a solution of N-succinimidyl 3-maleimidopropionate (5.0 g, 19 mmol) and L-alanyl-L-alanine (3.4 g, 21 mmol) in DMF (25 mL) was added DIPEA (3.1 mL, 18 mmol) and the mixture was stirred at rt overnight. The reaction mixture was concentrated under reduced pressure to give a residue, which was purified by silica gel chromatography eluting with 2% acetic acid in EtOAc to give the title compound (4.0 g, 69%). ES/MS m/z 312.3 (M+H).

### PREPARATION 8

### 3-(2,5-Dioxo-2,5-dihydro-1H-pyrrol-1-yl)-N—((S)-1-(((S)-1-((3-((2-fluoro-3-((6aR,6b S,7S,8aS,8b S,10S,11aR,12aS,12b S)-7-hydroxy-8b-(2-hydroxyacetyl)-6a, 8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)-4-methylphenoxy)methyl)phenyl)amino)-1-oxopropan-2-yl)amino)-1-oxopropan-2-yl)propanamide (Herein Also Referred to as “GC-L”)

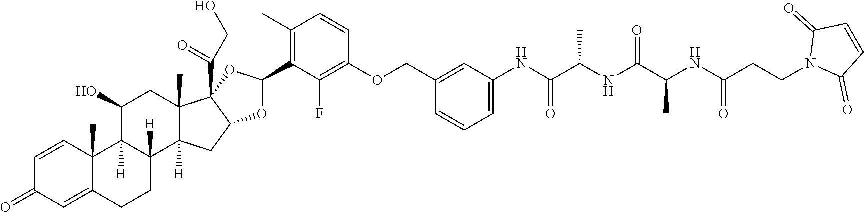

To a solution of (6aR,6bS,7S,8aS,8bS,10S,11aR,12aS,12bS)-10-(3-((3-aminobenzyl)oxy)-2-fluoro-6-methylphenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (GC1, 24 g, 39 mmol, see Preparation 6) and 3-(2,5-dioxo-2,5-dihydro-1H-pyrrol-1-yl)propanoyl)-L-alanyl-L-alanine (15 g, 47 mmol, see Preparation 7) in DMF (250 mL), cooled to 0-5° C., was added 2,6-lutidine (11 mL, 97 mmol) followed by HATU (17 g, 43 mmol). The mixture was stirred at 0-5° C. for 5 min, then the cooling bath was removed, and the mixture was stirred for 2 h. The mixture was diluted with EtOAc. The organic solution was washed with three portions water, one portion satd aq NaCl, dried over Na₂SO₄ (MeOH added to aid solubility), filtered and evaporated to give the crude product. The crude product was purified by silica gel chromatography using a gradient of 1-10% MeOH in DCM to give the title compound (24 g, 68%). ES/MS m/z 911.4 (M+H). 1H NMR (400.13 MHz, DMSO): δ 9.88 (s, 1H), 8.20 (d, J=7.1 Hz, 1H), 8.11 (d, J=7.2 Hz, 1H), 7.68 (s, 1H), 7.60-7.58 (m, 1H), 7.34-7.29 (m, 2H), 7.14-7.09 (m, 2H), 7.00 (s, 2H), 6.89 (d, J=8.4 Hz, 1H), 6.34 (s, 1H), 6.18 (dd, J=1.8, 10.0 Hz, 1H), 5.95 (s, 1H), 5.76 (s, 1H), 5.31 (d, J=6.8 Hz, 1H), 5.13-5.04 (m, 3H), 4.78 (d, J=3.1 Hz, 1H), 4.41-4.30 (m, 4H), 4.10-4.00 (m, 1H), 3.61 (t, J=7.3 Hz, 2H), 2.42-2.31 (m, 3H), 2.22 (s, 3H), 2.11-2.01 (m, 2H), 1.91-1.78 (m, 5H), 1.40 (s, 3H), 1.31 (d, J=7.2 Hz, 3H), 1.19-1.11 (m, 5H), 0.88 (s, 3H).

### PREPARATION 9

### 3-(2,5-dioxo-2,5-dihydro-1H-pyrrol-1-yl)-N—((S)-1-((S)-1-((3-((2-fluoro-3-((6aR,6b S,7S,8aS,8b S,10R,11aR,12aS,12b S)-7-hydroxy-8b-(2-hydroxyacetyl)-6a, 8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)-4-methylphenoxy)methyl)phenyl)amino)-1-oxopropan-2-yl)amino)-1-oxopropan-2-yl)propanamide

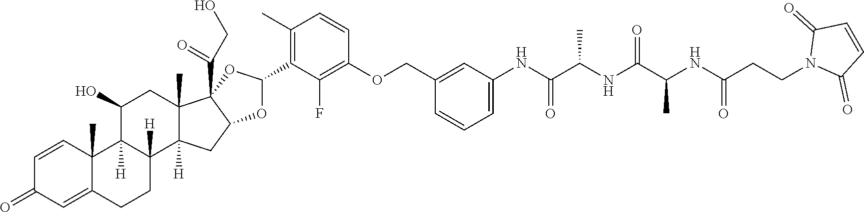

In a manner analogous to the procedure described in Preparation 8, the compound of Preparation 9 was prepared from (6aR,6bS,7S,8aS,8bS,10R,11aR,12aS,12bS)-10-(3-((3-aminobenzyl)oxy)-2-fluoro-6-methylphenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (see Preparation 5) and 3-(2,5-dioxo-2,5-dihydro-1H-pyrrol-1-yl)propanoyl)-L-alanyl-L-alanine (see Preparation 7). ES/MS m/z 911.4 (M+H). 1H NMR (500.11 MHz, DMSO): δ 9.88 (s, 1H), 8.23-8.20 (m, 1H), 8.11 (d, J=7.2 Hz, 1H), 7.69 (s, 1H), 7.59 (d, J=8.0 Hz, 1H), 7.33-7.28 (m, 2H), 7.15-7.08 (m, 2H), 7.00 (s, 2H), 6.91-6.89 (m, 1H), 6.17 (dd, J=1.7, 10.1 Hz, 1H), 5.94 (s, 1H), 5.61 (s, 1H), 5.16-5.12 (m, 3H), 4.98-4.96 (m, 1H), 4.81 (d, J=3.1 Hz, 1H), 4.49-4.36 (m, 6H), 3.61 (t, J=7.3 Hz, 2H), 2.41 (t, J=7.3 Hz, 2H), 2.30-2.29 (m, 4H), 2.17-2.15 (m, 2H), 1.88-1.77 (m, 4H), 1.69-1.61 (m, 1H), 1.40 (s, 3H), 1.31 (d, J=7.2 Hz, 3H), 1.18 (d, J=7.2 Hz, 3H), 0.93-0.87 (m, 6H).

### EXAMPLES

### Example 1. Generation of the Anti-Human IL-4Rα Antibody GC Conjugates

### Example 1a. Generation and Engineering of Anti-Human IL-4Rα Antibodies

Antibody generation: To develop antibodies specific to human IL-4Rα, transgenic mice with human immunoglobulin variable regions were immunized with Fc-tagged extracellular domain (ECD) of human IL-4Rα and boosted, alternately, with human and cynomolgus monkey Fc-tagged IL-4Rα ECD proteins. Screening was done with histidine-tagged human and cynomolgus monkey IL-4Rα ECD to identify cross reactivity and in the absence or presence of excess soluble IL-4 to identify IL-4 blocking antibodies. Cross reactive antibodies were cloned as Fabs, expressed, and purified by standard procedures, and tested in a reporter cell line, Human Embryonic Kidney (HEK)-Blue IL-4/IL-13 (InvivoGen) for blocking activity to IL-4 and IL-13. Antibodies were selected and engineered in their CDRs, variable domain framework regions, and IgG isotype to improve characteristics such as, affinity, stability, solubility, viscosity, hydrophobicity, as well as reduced aggregation.

The amino acid sequence of human IL-4Rα ECD is provided by SEQ ID NO: 15, the amino acid sequence of cynomolgus monkey IL-4Rα ECD is provided by SEQ ID NO: 16, the amino acid sequence of human IL-4 is provided by SEQ ID NO: 17, and the amino acid sequence of human IL-13 is provided by SEQ ID NO: 18.

The antibodies of the invention can be synthesized and purified by well-known methods. An appropriate host cell, such as Chinese hamster ovarian cells (CHO), can be either transiently or stably transfected with an expression system for secreting antibodies using a predetermined HC:LC vector ratio if two vectors are used, or a single vector system encoding both heavy chain and light chain. Clarified media, into which the antibody has been secreted, can be purified using the commonly used techniques.

Antibody engineering for affinity and biophysical properties: IL-4Rα antibody Ab10 was engineered as a Fab in mammalian cell expression vectors using a high-throughput, site-specific, saturation mutagenesis protocol to find mutations that improve affinity and/or biophysical properties (such as, stability, solubility, viscosity, hydrophobicity, aggregation, serum protein binding, thermal, or chemical stability).

Briefly, CDR mutagenesis and atypical germline residues in the framework regions of the Ab10 were assessed. CDR analysis identified key CDR substitutions: LCDR3H91W, N92S, which significantly improved affinity of the resulting antibody from the 10⁻⁹ M range to the 10⁻¹¹ M range. Further analysis and experimentation identified key residue modifications in the VH as follows: A23V, N92S, I31H; VL: G28D which improved thermal stability in the thermal challenge ELISA while maintaining affinity. Additionally, amino acid residue substitutions in the VH: A23V, I58V; VL: G28D were found to reduce self-association and hydrophobicity while maintaining affinity. Amino acid residue substitution: VH: I31H was found to reduce serum protein binding. Certain antibodies were further engineered to eliminate deaminidation by substituting the asparagine in the HC Framework 3 (N72) to Aspartic acid (N72D). In summary, 7 key amino acid residues were identified and engineered into Ab1, Ab2, Ab3, Ab4, Ab5, Ab6, Ab7 and Ab8 as follows in the VH: A23V, I31H, I58V, N72D and in the VL: G28D, H91W, N92S which significantly improved affinity and biophysical properties (as shown below) such as, thermal stability, reduced self-association, hydrophobicity, and/or serum protein binding of the anti-human IL-4Rα antibodies, without negatively impacting other functional or biophysical properties of the antibodies. Ab9 was engineered with amino acid residue substitutions as follows in the VH: I31H, I58V, N72D and in the VL: H91W, N92S. Table 2 shows the CDR amino acid sequences of the exemplified antibodies. The exemplified antibodies were generated with different IgG backbones including those as provided in Table 3.

Antibody hinge and Fc backbone selection: The anti-human IL-4Rα antibodies Ab1, Ab2, Ab3, Ab4, Ab5, Ab9 and Ab10, were engineered to include the S228P mutation, which stabilizes the hinge and prevents arm exchange. A wild type IgG4 domain along with a human kappa constant domain was used to complete the construct. The antibodies were synthesized, expressed, and purified essentially as described above.

Human IgG1A and/or human IgG4P backbone were selected for the exemplified antibodies because they provided an unexpected advantage of binding to B cells and myeloid cells. As shown below, the exemplified antibodies were found to have greater binding potency to B cells, when compared to the effector null antibody, thus indicating that the Fc portion of the exemplified antibody that is not engineered to be effector null positively impacted B cell binding of the anti-IL-4Rα antibodies.

Antibody constant region engineering to improve viscosity: The anti-human IL-4Rα antibody heavy chain constant region was further engineered through charge balancing to improve viscosity and mitigate potential electrostatic interaction between the Fab and constant domains of the antibody. 5 key amino acid residues in the CH1, CH2, and CH3 domains in the IgG4 were identified as impacting the viscosity of the anti-human IL-4Rα antibodies: 1) E137 (CH1 domain), 2) D203 (CH1 domain), 3) Q274 (CH2 domain), 4) Q355 (CH3 domain), and 5) E419 (CH3 domain). Antibodies with the various combinations of these amino acid substitutions were generated, including the combination: Q274K (CH2 domain), Q355R (CH3 domain), and E419Q (CH3 domain) for Ab1, to significantly improve viscosity of the antibody. The analogous positions in a hIgG1 constant region for the 5 amino acids are different and were found to impact the overall pI of each domain.

TABLE 2
CDR amino acid sequences of exemplified
anti-human IL-4Rα antibodies

| IL-4Rα | CDR Sequence | | | | | |
|---|---|---|---|---|---|---|
| Antibody | HCDR1 | HCDR2 | HCDR3 | LCDR1 | LCDR2 | LCDR3 |
| Ab1, Ab2, | SEQ ID | SEQ ID | SEQ ID | SEQ ID | SEQ ID | SEQ ID |
| Ab3, Ab4, | NO: 1 | NO: 2 | NO: 3 | NO: 4 | NO: 5 | NO: 6 |
| Ab5, Ab6, | | | | | | |
| Ab7, Ab8 | | | | | | |
| Ab9 | SEQ ID | SEQ ID | SEQ ID | SEQ ID | SEQ ID | SEQ ID |
| | NO: 42 | NO: 2 | NO: 3 | NO: 22 | NO: 5 | NO: 6 |
| Ab10 | SEQ ID | SEQ ID | SEQ ID | SEQ ID | SEQ ID | SEQ ID |
| | NO: 19 | NO: 20 | NO: 3 | NO: 22 | NO: 5 | NO: 24 |

TABLE 3
Amino Acid sequences of exemplified
anti-human IL-4Rα antibodies

| IL-4Rα | | | | |
|---|---|---|---|---|
| Antibody | HC | LC | VH | VL |
| Ab1 | SEQ ID NO: | SEQ ID NO: | SEQ ID NO: | SEQ ID NO: |
| | 9 | 10 | 7 | 8 |
| Ab2 | SEQ ID NO: | SEQ ID NO: | SEQ ID NO: | SEQ ID NO: |
| | 50 | 10 | 7 | 8 |
| Ab3 | SEQ ID NO: | SEQ ID NO: | SEQ ID NO: | SEQ ID NO: |
| | 37 | 10 | 7 | 8 |
| Ab4 | SEQ ID NO: | SEQ ID NO: | SEQ ID NO: | SEQ ID NO: |
| | 31 | 10 | 7 | 8 |
| Ab5 | SEQ ID NO: | SEQ ID NO: | SEQ ID NO: | SEQ ID NO: |
| | 35 | 10 | 7 | 8 |
| Ab6 | SEQ ID NO: | SEQ ID NO: | SEQ ID NO: | SEQ ID NO: |
| | 33 | 10 | 7 | 8 |
| Ab7 | SEQ ID NO: | SEQ ID NO: | SEQ ID NO: | SEQ ID NO: |
| | 13 | 10 | 7 | 8 |
| Ab8 | SEQ ID NO: | SEQ ID NO: | SEQ ID NO: | SEQ ID NO: |
| | 52 | 10 | 7 | 8 |
| Ab9 | SEQ ID NO: | SEQ ID NO: | SEQ ID NO: | SEQ ID NO: |
| | 46 | 47 | 44 | 45 |
| Ab10 | SEQ ID NO: | SEQ ID NO: | SEQ ID NO: | SEQ ID NO: |
| | 27 | 28 | 25 | 26 |

### Example 1b. Generation of Anti-Human IL-4Rα Ab1 GC Conjugate (n=4)

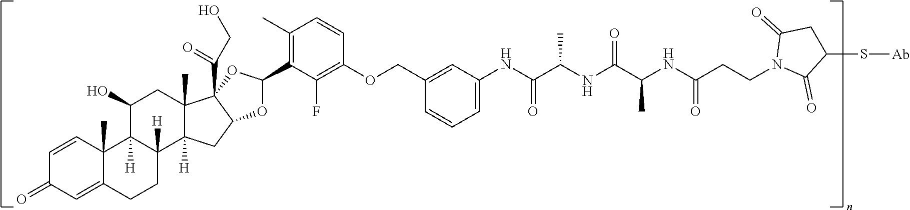

wherein n is 4; and

### Ab is Ab1.

The exemplified anti-human IL-4Rα Ab1 (see Table 2 and Table 3) was first reduced in the presence of 40-fold molar excess of dithiothreitol (DTT) for 2 hours at 37° C. or >16 hours at 21° C. This initial reduction step was used to remove the various capping groups, including cysteine and glutathione which are bond to the engineered cysteine at the 124 and 378 position of the heavy chain during expression. Following the reduction step, the sample was purified through a desalting resin to remove the cysteine caps as well as the reducing agent. A subsequent 2-hour oxidation step was carried out at room temperature (˜21° C.) in the presence 10-fold molar excess of dehydroascorbic acid (DHAA) to reform the native interchain disulfides between the light chain and heavy chain as well as the pair of hinge disulfides. After the 2 hour oxidation step, 4-8 molar equivalents of the glucocorticoid receptor agonist payload-linker (“GC-L”), 3-(2,5-dioxo-2,5-dihydro-1H-pyrrol-1-yl)-N—((S)-1-(((S)-1-((3-((2-fluoro-3-((6aR,6b S,7S,8aS,8b S,10S,11aR,12a5,12b S)-7-hydroxy-8b-(2-hydroxyacetyl)-6a, 8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)-4-methylphenoxy)methyl)phenyl)amino)-1-oxopropan-2-yl)amino)-1-oxopropan-2-yl)propanamide prepared in Preparation 8, was added using a 10 mM stock solution solubilized in DMSO. The sample was then incubated at room temperature for 30-60 minutes to allow for efficient conjugation of the LP to the engineered cysteines. A subsequent polishing step, such as Size Exclusion Chromatography (SEC) or Tangential Flow Filtration (TFF) was then used to buffer exchange the sample into an appropriate formulation buffer and to remove DMSO and any excess linker-payload.

Drug to antibody ratio (DAR) assessment: To assess the average number of linker-payloads present on the final conjugates, two analytical methods were used, which included: 1) Reverse phase (RP) HPLC and 2) Time of Flight (TOF) mass spectrometry. Both methods required an initial sample reduction step, which included the additional of dithiothreitol (DTT) to a final concentration of ˜10 mM, followed by a 5-minute incubation at 42° C.
Reverse Phase HPLC Method: 1 mg/mL of the anti-human IL-4Rα antibody GC conjugate sample was reduced by incubating the sample at 42° C. in the presence of 10 mM DTT for 5 minutes. Ten to thirty micrograms of the reduced anti-human IL-4Rα antibody GC conjugate sample was injected onto a Phenyl SPW, 4.6 mm×7.5 cm, 10 μM column (Tosh Part #0008043). The A buffer was made up of 0.1% trifluoroacetic acid (TFA) in water while B buffer was comprised of 0.1% trifluoroacetic acid (TFA) in acetonitrile (ACN). The column was equilibrated in 20% B buffer prior to sample injection followed by a gradient from 28% B to 40% B over ˜8.5 column volumes. The average DAR was determined by calculating the contribution from each individual DAR species from the fractional percentage multiplied by the DAR number for each contributing species. As this value is based on a reduced sample and only represents half of the molecule, the number was then multiplied by 2 to account for an intact antibody GC conjugate. DAR calculations for the anti-human IL-4Rα Ab1 GC conjugate of Example 1b are provided in Table 4.

TABLE 4
Quantification of the average DAR for the eCys conjugate
of Example 1b using fractional percentages for
each DAR species from a reduced sample.

| DAR | Peak % | DAR Contribution |
|---|---|---|
| 0 | 24.985 | 0.00 |
| 1 | 1.939 | 0.07 |
| 2 | 0 | 0.00 |
| 3 | 0 | 0.00 |
| 4 | 0 | 0.00 |
| 5 | 0 | 0.00 |
| Total LC % | 26.924 | |
| | LC Avg DAR | 0.07 |
| | | (DAR contribution from LC) |
| 0 | 0.417 | 0.00 |
| 1 | 12.505 | 0.17 |
| 2 | 46.384 | 1.27 |
| 3 | 13.346 | 0.55 |
| 4 | 0.423 | 0.02 |
| Total HC % | 73.075 | |
| | HC Avg DAR | 2.01 |
| | | (DAR contribution from HC) |
| (HC + LC)2 | Final Avg DAR | 4.17 |

Time of Flight Mass Spectrometry Method: 8 μg of the reduced sample was injected onto a Poroshell 300sb-C3 2.1×2.5 mm, 5 μM column (Agilent Part #821075-924). Buffer A was made up of 0.1% trifluoroacetic acid (TFA) in water while buffer B comprised of 0.1% trifluoroacetic acid (TFA) in acetonitrile (ACN). The column was equilibrated in 0% B buffer prior to sample injection followed by a gradient from 10% B to 80% B over ˜28 column volumes. The average DAR was determined by calculating the contribution from each individual DAR species from the fractional percentage multiplied by the DAR number for each contributing species. As this value is based on a reduced sample and only represents half of the molecule, the number was then multiplied by 2 to account for an intact antibody. DAR calculations for the anti-human IL-4Rα Ab1 GC conjugate of Example 1b are provided in Table 5.

TABLE 5
Quantification of the average DAR for the eCys conjugate of
Example 1b using fractional percentages based on total ion
counts from Time of Flight mass spectrometry analysis.

| DAR | Ion counts | DAR Contribution |
|---|---|---|
| 0 | 79479.5 | 0.00 |
| 1 | 880.32 | 0.01 |
| 2 | 0 | 0.00 |
| 3 | 0 | 0.00 |
| 4 | 0 | 0.00 |
| 5 | 0 | 0.00 |
| Total LC % | 80359.82 | |
| | LC Avg DAR | 0.01 |
| | | (DAR contribution from LC) |
| 0 | 915.88 | 0.00 |
| 1 | 11501.86 | 0.17 |
| 2 | 50152.76 | 1.45 |
| 3 | 6269.16 | 0.27 |
| 4 | 499.08 | 0.03 |
| Total HC % | 69338.74 | |
| | HC Avg DAR | 1.91 |
| | | (DAR contribution from HC) |
| (HC + LC)2 | Final Avg DAR | 3.85 |

### Example 1c. Generation of Anti-Human IL-4Rα Ab1 GC Conjugate (n=3)

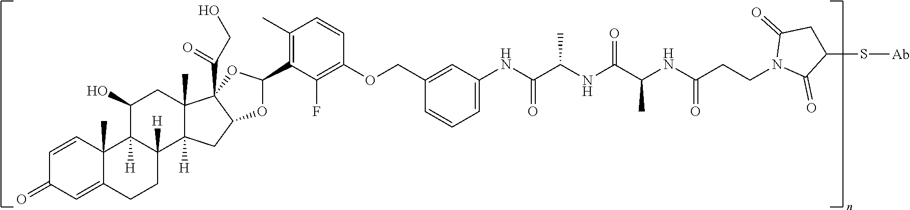

wherein n is 3; and

### Ab is Ab1.

The conjugate of Example 1c is prepared in a manner analogous to the procedure described in Example 1b using a lower molar ratio of the GC-L, 3-(2,5-dioxo-2,5-dihydro-1H-pyrrol-1-yl)-N—((S)-1-(((S)-1-((3-((2-fluoro-3-((6aR,6b S,7S,8aS,8b S,10S,11aR,12aS,12b S)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)-4-methylphenoxy)methyl)phenyl)amino)-1-oxopropan-2-yl)amino)-1-oxopropan-2-yl)propanamide to Ab1 during the conjugation step. For example, use of a molar ratio of the corresponding GC-L:Ab1 of 3.2:1 will result in a final DAR of approximately 3.

Example 1d. Thiosuccinimide Hydrolysis: The thiosuccinimide ring of the compound Formula Ie, can be hydrolyzed under conditions well known in the art as shown in Scheme 1 below (See, e.g., WO 2017/210471, paragraph 001226) to provide the ring-opened product of Formula If.

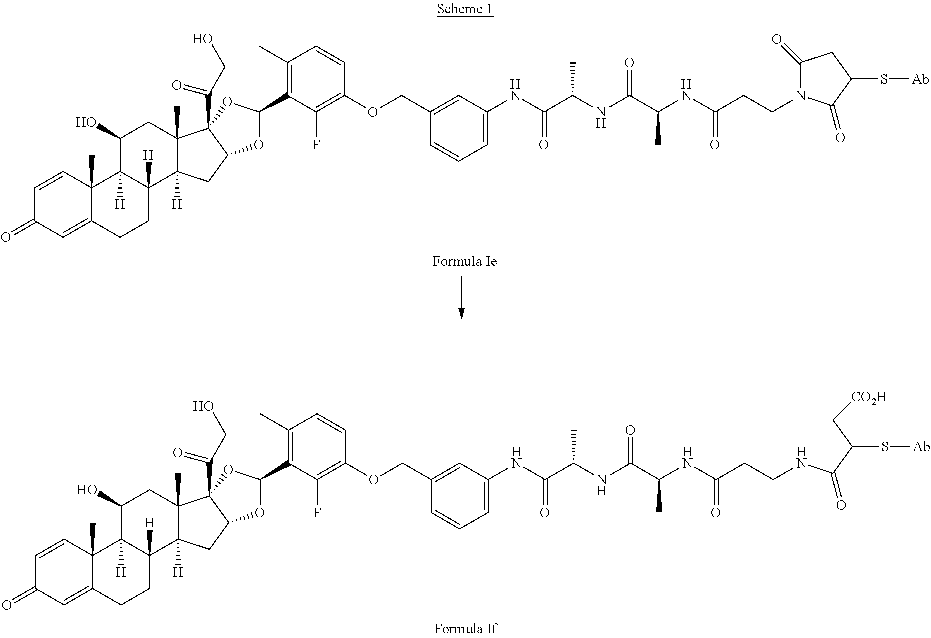

In addition, the above thiosuccinimide ring of the compound of Formula Ie may undergo at least partial hydrolysis in vivo and under standard or well-known formulation conditions to provide the ring-opened product of Formula If.

### Example 2. Structural and Functional Epitope Determination of the Anti-Human IL-4Rα Antibodies

Example 2a. Structural epitope of Ab9 Fab by X-ray crystallography. The physical epitope of the Fab of the anti-IL-4Rα Ab9 on human IL-4Rα was determined by identifying the interacting interfaces between human IL-4Rα and the exemplified antibodies. Briefly, to determine the structural epitope, human IL-4Rα ECD was co-crystallized with a Fab portion of Ab9. The structure of the Ab9 Fab complexed with IL-4Rα was determined by creating a hexahistidine tagged IgG1 variant of the heavy chain truncated after the CH1 domain and the “Crystal Kappa” version of the light chain of the Ab9 Fab (see, Lieu et al., “Rapid and Robust Antibody Fab Fragment Crystallization Utilizing Edge-to-edge Beta-sheet Packing,” PLoS One, 15(9) (2020), which is herein incorporated by reference in its entirety). The Ab9 variant was co-expressed with a hexahistidine tagged version of human IL-4Rα ECD containing a C182L mutation, the complex was then purified by immobilized metal affinity chromatography and screened using standard commercially available screens for crystallization. Crystals were obtained and x-ray diffraction data was collected at the Advanced Photon Source. The diffraction data was reduced and solved by molecular replacement and refined to yield a 2.8 angstrom structure of the exemplified Ab9 Fab and IL-4Rα ECD complex. From the resulting crystal structure, any IL-4Rα amino acid residues within 4.5 angstroms of an atom of the co-crystallized Ab9 Fab was counted as part of the epitope (using PyMOL visualization software [Schrödinger®]).

The PyMOL analysis demonstrated that the IL-4Rα amino acid residues (with respect to SEQ ID NO: 15) that are within 4.5 angstroms of the Ab9 Fab in the crystal structure complex comprise of the structural epitope for the exemplified antibodies. Specifically, the analysis determined the structural epitope comprises the following amino acid residues: Asp at position 12, Met at position 14, Ser at position 15, Ile at position 16, Tyr at position 37, Leu at position 39, Phe at position 41, Leu at position 43, Glu at position 45, His at position 47, Thr at position 48, Cys at position 49, Ile at position 50, His at position 62, Leu at position 64, Met at position 65, Asp at position 66, Asp at position 67, Val at position 69, Asp at position 72, Arg at position 99, Pro at position 121, Pro at position 123, Pro at position 124, Asp at position 125. The analysis further determined that the structural epitope spans domains 1 and 2 of the N-terminus fibronectin type-III domain of the IL-4Rα. Furthermore, the analysis determined that the following amino acid residues of the structural epitope are located in domain 2 of the N-terminal fibronectin type-III domains of the IL-4Rα: R99, P121, P123, P124, D125.

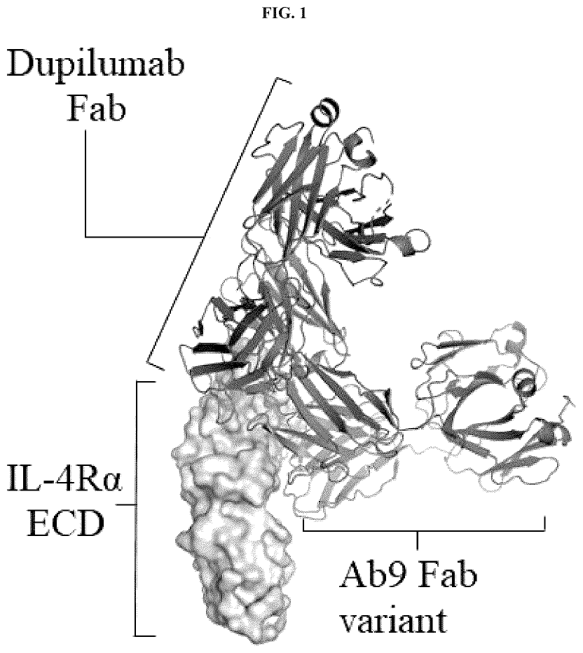

In addition, overlay of the exemplified Ab9 Fab and the crystal structure of a dupilumab Fab with the crystal kappa design complexed with human IL-4Rα (pdb accession code 6WGL) indicated that the Ab9 Fab bound to a novel epitope on IL-4Rα when compared to dupilumab (FIG. 1).

Furthermore, an alignment of the exemplified IL-4Rα Ab9 Fab:IL-4Rα complex crystal structure with published complexes of IL-4 and IL-13 and their respective receptors (pdb accession codes 3BPN and 3BPO) on the IL-4Rα component in each structure (using PyMOL visualization software) showed that the exemplified Ab9 Fab antibody epitope overlapped with both the IL-4 and the IL-13 binding sites to IL-4Rα, thus indicating that binding of the exemplified antibodies to IL-4Rα would physically block the IL-4 and IL-13 cytokines from binding to IL-4Rα when the Fab variant portion of the exemplified antibodies is bound to IL-4Rα.

Example 2b. Functional epitope of Ab10. The functional epitope of the exemplified anti-human IL-4Rα Ab10 was determined by ELISA. Briefly, thirty surface amino acid residue substitutions were introduced individually into hexahistidine tagged human IL-4Rα extra cellular domain (ECD) as follows: K2D, E6R, K22D, P26R, T31R, F41A, L42G, L43G, E45R, G56R, D66R, A71R, Q82G, K87D, E94R, H107A, D108R, P124R, D125R, D143R, R148D, L155R, R160D, S164R, S168R, Q181R, P192R, K195D, or H197G. Each mutant protein having a single amino acid residue substitution as described above was transiently expressed in CHO cells and purified using standard immobilized metal affinity chromatography techniques. ELISA plates were coated with 1 μg/mL goat anti-human kappa antibody (Southern Biotech, Cat #2060-01) in PBS at 4° C. overnight, then washed 3 times in PBST and blocked with PBS casein for 30 min at room temperature. The plates were then washed 3 times with PBST and the exemplified anti-human IL-4Rα antibody Ab6 was added to the wells at a final concentration of 1 μg/mL in PBS-casein and incubated for 1 hour. The plates were washed 3 times with PBST, the IL-4Rα mutant proteins were serially diluted 3-fold from 1 μg/mL in PBS-casein and added to the plate at 50 μL/well and incubated for 1 hour at room temperature. The plates were washed 3 times with PBST and a 5000-fold dilution of anti-histidine tag antibody HRP conjugate (R&D Systems, Cat. #MAB050H) in PBS-casein was added and incubated for 1 hour at room temperature. The plates were washed 3 times, TMB substrate (Pierce, Cat. #34021) was added per manufacturer instructions, the reaction was quenched with H2504, and absorbance was read at 450 nm on an ELISA plate reader. The functional epitope of the antibody was determined as the mutated amino acid residues corresponding to the wells that showed no binding signal or showed a significantly reduced binding signal when compared to the control antibodies.

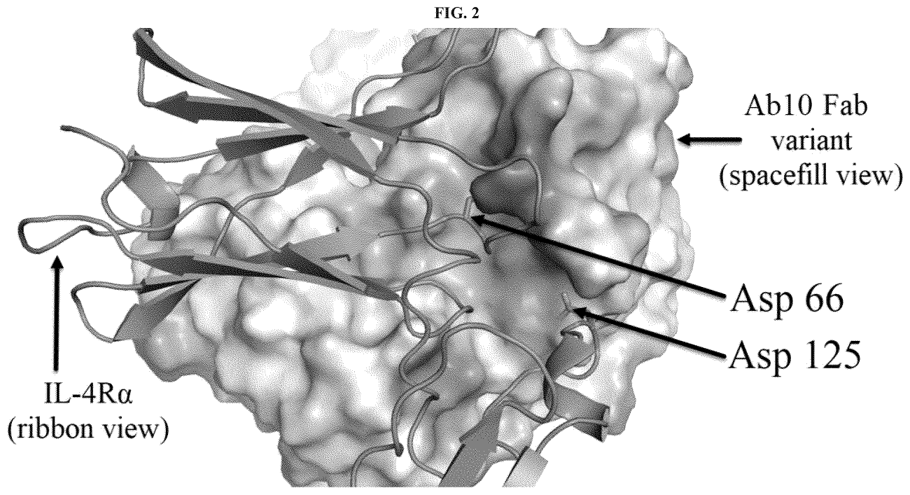

The results in Table 6A, show that the functional epitope for the exemplified anti-human IL-4Rα Ab10 comprises amino acid residues D66 and D125. Among the amino acid residues identified in the structural epitope, amino acid residue substitutions of D66R and D125R on the IL-4Rα displayed a significantly negative impact on binding of the Ab10 to the mutated IL-4Rα respectively. Specifically, substitution of amino acid residue D66 of the IL-4Rα to Arginine reduced binding of the Ab10 to the mutated IL-4Rα to below that of the control (0.04 OD₄₅₀ and 0.14 OD₄₅₀, respectively). Furthermore, substitution of amino acid residue D125 to Arginine, which is located near amino acid residue D66 on the crystal structure of the IL-4Rα (see FIG. 2), also showed significantly reduced binding of 0.59 OD₄₅₀. The remaining amino acid substitutions were either within the range of positive binding or outside of the determined structural epitope.

TABLE 6A
Functional epitope determination of exemplified
anti-human IL-4Rα antibody Ab10

| Amino acid substitution | ELISA (OD₄₅₀) |
|---|---|
| K2D | 1.48 |
| E6R | 1.08 |
| K22D | 0.64 |
| P26R | 1.40 |
| T31R | 1.61 |
| F41A | 1.53 |
| L42G | 1.20 |
| L43G | 1.69 |
| E45R | 1.15 |
| G56R | 1.43 |
| D66R | 0.04 |
| A71R | 1.35 |
| Q82G | 1.59 |
| K87D | 1.16 |
| E94R | 1.46 |
| H107A | 1.54 |
| T108R | 1.21 |
| V110R | 1.47 |
| P124R | 1.35 |
| D125R | 0.59 |
| D143R | 1.51 |
| R148D | 1.39 |
| L155R | 1.53 |
| R160D | 1.54 |
| S164R | 1.56 |
| S168R | 1.53 |
| Q181R | 1.30 |
| P192R | 1.33 |
| K195D | 1.06 |
| H197G | 1.02 |
| Buffer Control (no receptor) | 0.14 |

Example 2c. Structural epitope of Ab1 Fab by X-ray crystallography. The physical epitope of the Fab of the Ab1 anti-human IL-4Rα antibodies on human IL-4Rα was determined essentially as described above. Crystals were obtained and x-ray diffraction data was collected at the Advanced Photon Source. The diffraction data was reduced and solved by molecular replacement and refined to yield a 2.49 angstrom structure of the exemplified Fab and IL-4Rα ECD complex. From the resulting crystal structure, any IL-4Rα amino acid residues within 4.5 angstroms of an atom of the co-crystallized Fab was counted as part of the epitope (using Molecular Operating Environment (MOE) visualization, modeling and simulations software [Chemical Computing Group], Coot (General Public License) and PyMOL visualization software [Schrödinger®]).

The MOE, Coot, and PyMOL analysis demonstrated that the IL-4Rα amino acid residues (with respect to SEQ ID NO: 15) that are within 4.5 angstroms of the exemplified Fab in the crystal structure complex comprise of the structural epitope. Specifically, the analysis determined the structural epitope comprises the following amino acid residues: Asp at position 12, Met at position 14, Ser at position 15, Ile at position 16, Leu at position 39, Phe at position 41, Leu at position 42, Thr at position 48, Cys at position 49, Ile at position 50, Glu at position 52, His at position 62, Leu at position 64, Met at position 65, Asp at position 66, Asp at position 67, Val at position 68, Val at position 69, Asp at position 72, Arg at position 99, Pro at position 121, Pro at position 123, Pro at position 124, Asp at position 125, Pro at position 192. Asp at position 66 was well coordinated having interactions between 2.6-2.9 Å with the heavy chain of the Ab1 Fab. Asp at position 67 had longer range interactions between 3.1-3.5 Å and demonstrated flexibility in its binding position, as evidenced by the observance of excess density around its sidechain. The analysis determined that the structural epitope spans domains 1 and 2 of the N-terminus fibronectin type-III domain of the IL-4Rα. Furthermore, the analysis determined that the following amino acid residues of the structural epitope are located in domain 2 of the N-terminal fibronectin type-III domains of the IL-4Rα: R99, P121, P123, P124, D125, P192.

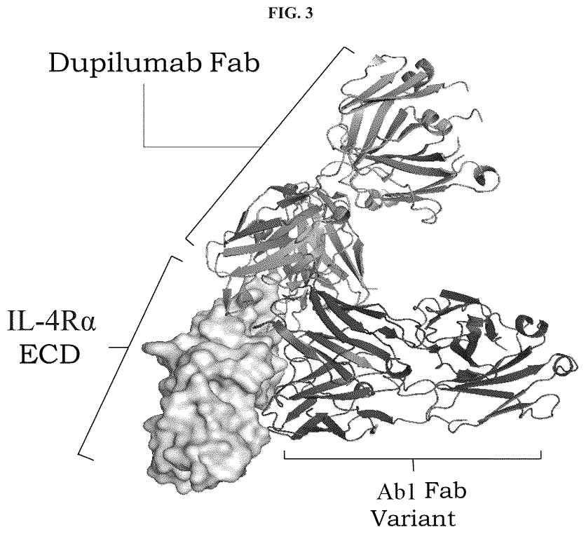

Overlay of the exemplified anti-human IL-4Rα Ab1 Fab and the crystal structure of a dupilumab Fab with the crystal kappa design complexed with human IL-4Rα (pdb accession code 6WGL) showed that the anti-human IL-4Rα Ab1 bound to a novel epitope on IL-4Rα when compared to dupilumab (FIG. 3).

Alignment of the exemplified anti-human IL-4Rα Ab1 Fab:IL-4Rα complex crystal structure with published complexes of IL-4 and IL-13 and their respective receptors (pdb accession codes 3BPN and 3BPO) on the IL-4Rα in each structure (using PyMOL visualization software) showed that the exemplified anti-human IL-4Rα Ab Fab antibody epitope overlapped with both the IL-4 and the IL-13 binding sites to IL-4Rα. This indicated that binding of the exemplified Ab1 would physically block the IL-4 and IL-13 cytokines from binding to IL-4Rα.

Example 2d. Functional epitope of Ab1. The functional epitope of the exemplified human IL-4Rα antibody Ab1 was determined by ELISA. Briefly, thirty surface amino acid residue substitutions were introduced individually into hexahistidine tagged human IL-4Rα extra cellular domain (ECD) as follows: K2D, E6R, K22D, P26R, T31R, F41A, L42G, L43G, E45R, E52R, G56R, D66R, A71R, Q82G, K87D, E94R, H107A, D108R, P124R, D125R, D143R, R148D, L155R, R160D, S164R, S168R, Q181R, P192R, K195D, or H197G. Each mutant protein having a single amino acid residue substitution as described above was transiently expressed in CHO cells and purified using standard immobilized metal affinity chromatography techniques. ELISA plates were coated with 1 μg/mL goat anti-human IgG Fc antibody (Jackson ImmunoResearch Laboratories, Cat #109-005-098) in PBS at 4° C. overnight, then washed 3 times in PBST and blocked with PBS casein for 1 hour at room temperature. The plates were then washed 3 times with PBST and the exemplified human IL-4Rα antibody was added to the wells at a final concentration of 1 μg/mL in PBS-casein and incubated for 1 hour at room temperature. The plates were washed 3 times with PBST, the IL-4Rα mutant proteins were serially diluted 5-fold from 1 μg/mL for 3 points. in PBS-casein and added to the plate at 50 μL/well and incubated for 1 hour at room temperature. The plates were washed 3 times with PBST and a 1000-fold dilution of anti-histidine tag antibody HRP conjugate (R&D Systems, Cat. #MAB050H) in PBS-casein was added and incubated for 45 minutes at room temperature. The plates were washed 3 times, TMB substrate (Pierce, Cat. #34028) was added per manufacturer instructions, the reaction was quenched with H₂SO₄, and absorbance was read at 450 nm on an ELISA plate reader. The functional epitope of the antibody was determined as the mutated amino acid residues corresponding to the wells that showed no binding signal or showed a significantly reduced binding signal when compared to the wild type control.

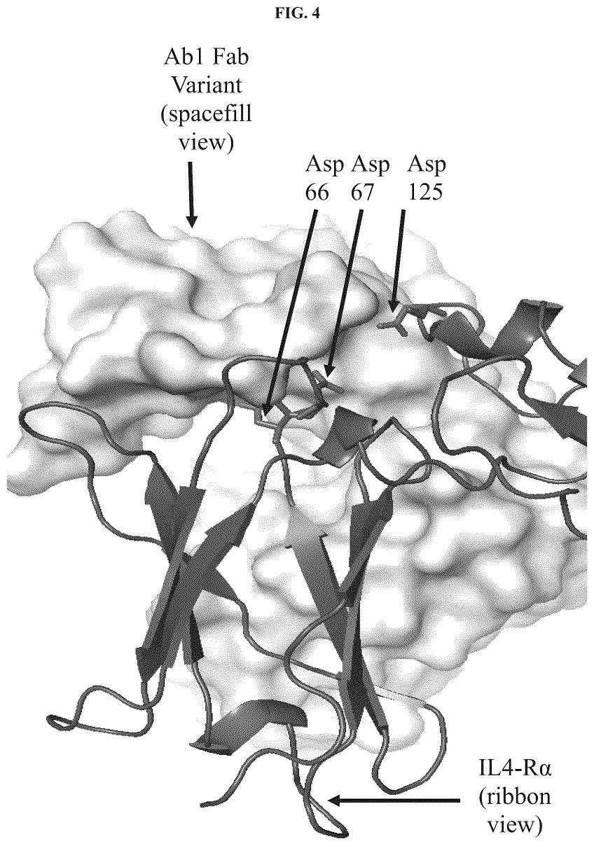

The results in Table 6B, show that the functional epitope for the exemplified anti-human IL-4Rα antibody Ab1 comprises amino acid residues D66. Amino acid residue substitution of D66 to D66R on the IL-4Rα reduced binding of the Ab1 to the D66R IL-4Rα to below that of the negative control (0.047 OD₄₅₀ and 0.063 OD 450, respectively). Amino acid residue D66 is located near structural epitope residues D67 and D125 in the crystal structure of the IL-4Rα (FIG. 4). The remaining amino acid substitutions were either within the range of positive binding or outside of the determined structural epitope.

TABLE 6B
Functional epitope determination of exemplified
anti-human IL-4Rα antibody Ab1

| Amino Acid | 1 μg/mL | 0.2 μg/ml | 0.04 μg/ml′ |
|---|---|---|---|
| Substitution | ELISA (OD₄₅₀) | ELISA (OD₄₅₀) | ELISA (OD₄₅₀) |
| K2D | 2.049 | 1.6064 | 1.0949 |
| E6R | 2.245 | 1.5049 | 0.9155 |
| K22D | 1.668 | 1.2977 | 0.5927 |
| P26R | 1.663 | 1.5241 | 0.8826 |
| T31R | 1.638 | 1.4901 | 0.9938 |
| F41A | 1.765 | 1.5448 | 1.1513 |
| L42G | 1.708 | 1.4848 | 0.9646 |
| L43G | 1.703 | 1.6986 | 1.3486 |
| E45R | 1.699 | 1.8614 | 1.5755 |
| E52R | 1.731 | 1.7126 | 1.0989 |
| G56R | 1.728 | 1.7032 | 1.1731 |
| D66R | 0.047 | 0.0425 | 0.0529 |
| A71R | 1.698 | 1.5651 | 0.9069 |
| Q82G | 1.742 | 1.5318 | 1.1029 |
| K87D | 1.748 | 1.5643 | 0.9115 |
| E94R | 1.733 | 1.6389 | 1.0337 |
| H107A | 1.889 | 1.8353 | 1.422 |
| T108R | 1.883 | 1.8157 | 1.4055 |
| P124R | 1.845 | 1.6964 | 1.1568 |
| D125R | 1.792 | 1.6089 | 1.0142 |
| D143R | 1.754 | 1.6923 | 1.2744 |
| R148D | 1.756 | 1.6005 | 1.0356 |
| L155R | 1.785 | 1.6802 | 1.1405 |
| R160D | 1.905 | 1.6992 | 1.2009 |
| S164R | 1.920 | 1.7747 | 1.4032 |
| S168R | 1.882 | 1.7138 | 1.1808 |
| Q181R | 1.909 | 1.7146 | 1.1669 |
| P192R | 1.939 | 1.749 | 1.2217 |
| K195D | 1.913 | 1.6531 | 0.992 |
| H197G | 1.851 | 1.6017 | 0.9666 |
| Wild type receptor | 1.910 | 1.8555 | 1.6475 |
| Buffer Control (no | 0.063 | 0.0625 | 0.0625 |
| receptor) | | | |

### Example 3. Binding Potency of Exemplified Anti-Human IL-4Rα Antibodies and IL-4Rα Ab1 GC Conjugate of Example 1b

Example 3a. Elisa Binding: The binding potency of the exemplified anti-human IL-4Rα Ab1 and IL-4Rα Ab1 GC conjugate to human and cynomolgus monkey IL-4Rα were measured using a competition Meso Scale Discovery (MSD) ELISA binding assay. A constant final concentration of Ab1 or the conjugate (10 pM) was mixed with a 3-fold dilution series of human or cynomolgus IL-4Rα to give a final starting concentration of 10 nM and the mix was incubated at 37° C. for 4 days. A 96-well multi-array plate (Meso Scale Diagnostics, Cat. #L15XA-3) was coated at 4° C. overnight with 0.5 μg/mL hexahistidine-tagged human or cynomolgus monkey IL-4Rα ECD in phosphate buffered saline (PBS). Following coating, plates were washed 10 times with 200 μL PBST (PBS with 0.05% Tween® 20) and blocked with 300 μL/well of PBS casein blocking buffer (Pierce, Cat. #37528) at 37° C. for 1 hour. Plates were then washed 10 times as above, and 50 μL of the preincubated antibody:IL-4Rα dilution series was transferred to the wells and incubated at 37° C. with 300 rpm shaking for 150 seconds. Plates were washed 10 times with PBST, 50 μL of 1 μg/mL anti-human antibody sulfo-tag 20 (Meso Scale Diagnostics, Cat. #R32AJ-1) was added to the plates for the assay of Table 7a, and anti-Human NHP Kappa Light chain SULFO-TAG 20 (Meso Scale Diagnostics, Cat #D20TF-6) was added to the plates for the assay of Table 7b, and plates were incubated at 37° C. with 300 rpm shaking for 30 minutes. Plates were washed 10 times with PBST, 150 μL/well of 1× Read Buffer T was added to the wells and analyzed on a SECTOR® Imager 6000 (Meso Scale Diagnostics) 15 min after buffer addition. The apparent K_{D} is determined by fitting a sigmoidal curve to the electrochemiluminescence (ECL) response vs. log (soluble IL-4Rα concentration) using GraphPad Prism 9. Data is graphed with normalized ECL values. The results in Table 7a are representative data from an individual experiment and the results in Table 7b is representative data from the mean of 3 independent experiments done in duplicate.

The exemplary results in Table 7a and 7b, show that exemplified anti-IL-4Rα antibodies and the Ab1 GC conjugate bind to both human and cynomolgus monkey IL-4Rα at comparable KID'S.

TABLE 7a
Examplary Binding affinities of exemplified anti-human IL-4Rα
antibodies to human and cynomolgus monkey IL-4Rα

| | IL-4Rα Species | K_{D} (pM) |
|---|---|---|
| Ab1 | Human | 20.82 |
| | Cynomolgus monkey | 23.32 |
| Ab4 | Human | 24.63 |
| | Cynomolgus monkey | 31.52 |
| Ab7 | Human | 45.08 |
| | Cynomolgus monkey | 38.88 |
| Ab10 | Human | 1993 |
| | Cynomolgus monkey | 1326 |

TABLE 7b
Examplary binding affinities of exemplified
anti-human IL-4Rα conjugate of Example
1b to human and cynomolgus monkey IL-4Rα

| | IL-4Rα Species | K_{D} (pM) |
|---|---|---|
| Ab1 | Human | 31.2 |
| | Cynomolgus monkey | 45.1 |
| Ab1 GC | Human | 24.7 |
| conjugate | Cynomolgus monkey | 45.8 |

Example 3b. Binding to B cells and T cells: Binding of the exemplified anti-human IL-4Rα antibodies to B cells and T cells was tested in a Fluorescence Activated Cell Sorting (FACS) assay. Human PBMCs were isolated from human blood samples by standard Ficoll-Paque™ plus (GE HEALTHCARE) density gradient centrifugation methods. Freshly isolated cells PBMCs were resuspended at 2×10⁶ cells/mL and allowed to rest for 15 minutes at room temperature, then plated at 100 μL/well into a round bottom 96-well plate (COSTAR®) and washed with FACS buffer (PBS containing 2% fetal bovine serum from Corning®). Exemplified anti-human IL-4Rα antibodies and the respective control IgG antibodies conjugated to Alexa Fluor® 647 according to manufacturer's protocol (Thermo Fisher Scientific) were added to the wells at 66.67 nM and diluted 4-fold in duplicate. Equivalent volume of 2× antibody cocktail containing: Human TruStain FcX™. FITC anti-human CD3 Antibody, Alexa Fluor® 700 anti-human CD4 Antibody (all from Biolegend®) and CD20 Monoclonal Antibody (2H7), PerCP-Cyanine5.5 (Thermo Fisher Scientific) was then added to the wells. Cells were incubated at 4° C. for 30 minutes, washed twice with FACS buffer and resuspended in a final volume of 100 μL FACS buffer. Viability dye, Sytox™ blue (Thermo Fisher Scientific), was added and the samples were analyzed via a flow cytometer (LSRFortessa™ X-20; BD BIOSCIENCES). Data analysis was performed using FlowJo software and statistical analysis was performed using GraphPad Prism 9. Data represents the mean±SEM of the percentage of IL-4Rα expressing cells from the CD20 B cell and CD4-positive T cell populations from six donors. Curves were generated by fitting a sigmoidal curve of the log (Ab concentration) vs. the percent of positive IL-4Rα expressing cells from the individual cell populations.

The results in Table 8A, from a representative experiment show that the exemplified anti-human IL-4Rα Ab1 and Ab7 bound IL-4Rα on the human PBMC isolated B cells (EC₅₀ of 0.15 nM and 0.14 nM, respectively) and CD4⁺ T cells (EC₅₀ of 26.3 nM and 28.7 nM respectively) with comparable affinities.

Furthermore, the results in Table 8B from a representative experiment comparing exemplified effector null anti-human IL-4Rα Ab8 to Ab7 showed that the effector null Ab8 had an unexpectedly reduced affinity to B cells (EC₅₀ of 1.07 nM) when compared to Ab7 (EC₅₀ of 0.27 nM), indicating that the Fc portion of the antibody may impact binding of the exemplified IL-4Rα antibody to B cells. Both Ab7 and Ab8 share the same CDR amino acid sequences.

TABLE 8A
Binding of exemplified anti-human
IL-4Rα antibodies to B and T cells

| | B Cells | T Cells |
|---|---|---|
| | EC₅₀ (nM) | EC₅₀ (nM) |
| Ab1 | 0.15 | 26.3 |
| Ab7 | 0.14 | 28.7 |

TABLE 8B
Binding of exemplified human IL-4Rα antibodies to B cells

| | B Cells |
|---|---|
| | EC₅₀ (nM) |
| Ab7 | 0.27 |
| Ab8 | 1.07 |

### Example 4. In Vitro Functional Characterization of the Anti-Human IL-4Rα Ab GC Conjugates

Example 4a. Cell based IL-4 and IL-13 cytokine blocking activity by the anti-human IL-4Rα Ab1: Antagonist activity of the exemplified anti-human IL-4Rα antibodies towards IL-4 and IL-13 was conducted with HEK-Blue IL-4R and IL-13R expressing cell line (InvivoGen) by measuring secreted embryonic alkaline phosphatase (SEAP) activity. HEK-Blue cells were plated overnight at 5×10 4 cells/well in 50 μL of growth media in a poly-lysine coated plate. Anti-human IL-4Rα antibodies were prepared in a Greiner 96-well low protein binding plate at 4-fold dilutions starting from 20 μg/mL in growth media. The dilution series was mixed with an equal volume of either recombinant human IL-4 or IL-13 (Eli Lilly) in growth media. 50 μL of the mixture was then added to the plates with the HEK-Blue cells to a final concentration of 100 pg/mL human IL-4 or 10 ng/mL human IL-13, and plates were then incubated overnight in a tissue culture incubator at 37° C. 20 μL of supernatant from the overnight incubated plates was transferred to a 96-well tissue culture treated plate and 180 μL per well of QUANTI-Blue™ (InvivoGen) was added, and the mixture was incubated for 45 min at 37° C. Secreted embryonic alkaline phosphatase (SEAP) activity was measured by at 650 nm on a SpectraMax microplate reader (Molecular Devices). Results were reported as optical density (OD) at 650 nm and statistical analysis was performed using GraphPad Prism 9. IC₅₀, and curves were generated by fitting a sigmoidal curve of the log (Ab concentration) vs. OD at 650 nm for each exemplified antibody.

The results showed that exemplified anti-human IL-4Rα antibodies Ab1, Ab7, and Ab9 inhibited both IL-4 and IL-13 induced SEAP activity in a dose dependent manner with IC₅₀'s of 0.08 nM, 0.07 nM and 0.03 nM respectively for IL-4 inhibition, and IC₅₀'s of 0.67 nM, 0.51 nM, and 0.24 nM respectively for IL-13 inhibition (Table 9).

TABLE 9
Cell based IL-4 and IL-13 inhibition by
exemplified anti-human IL-4Rα antibodies

| | IL-4 | IL-13 |
|---|---|---|
| | IC₅₀ (nM) | IC₅₀ (nM) |
| Ab1 | 0.08 | 0.67 |
| Ab7 | 0.07 | 0.51 |
| Ab9 | 0.03 | 0.24 |
| Ab10 | 3.45 | >35 |

Example 4b. Internalization of anti-human IL-4Rα Ab1 GC conjugate: The ability of the exemplified anti-human IL-4Rα Ab1 GC conjugate of Example 1b to bind IL-4Rα and internalize into Daudi cells was assessed. A pH sensitive label pHrodo™ iFL Microscale Labeling Kit (Invitrogen #P36014) was used to label 100 μg F(ab′)2 goat anti-human IgG Fcγ (Jackson Immuno Research Labs #109-006-098), according to manufacturer's protocol. Daudi cells (ATCC #CCL-213) were resuspended at 1×10⁶ cells/mL in media and 100 μL was seeded into a 96-well plate. The exemplified anti-human IL-4Rα Ab1 GC conjugate and anti-human IL-4Rα Ab were incubated with the pHrodo-labeled F(ab′)2 goat anti-human IgG at equal concentrations for 30 minutes at 4° C. for complex formation, then serially diluted by 10 for a 3-point curve and added to the Daudi cells and incubated for 25 hr at 37° C. in a CO₂ incubator. Following incubation, plates were spun down at 400×g for 3 minutes and supernatants were aspirated. Antibody cocktail containing CD20 Monoclonal Antibody (2H7), PerCP-Cyanine5.5 (eBioscience™) and Human TruStain FcX™ (BioLegend) in d-PBS with 2% Fetal Bovine Serum was added to wells and mixed by pipetting. Stained cells were then incubated for 30 minutes at 4° C. Cells were then washed with 100-150 μL of d-PBS with 2% FBS 2-3 times. Cells were resuspended in 100 μL d-PBS with 2% FBS and 5 μL of SYTOX™ Blue Dead Cell Stain (Invitrogen) was added to appropriate wells and mixed by pipetting. Plates were analyzed on the BD LSRFortessa™ X-20 Cell Analyzer. Analysis was performed using FlowJo software. Curves were generated by plotting concentration vs. the pHrodo geometric mean fluorescence intensity (gMFI) using GraphPad Prism 9.

The results in Table 10 show that the anti-human IL-4Rα Ab1 GC conjugate of Example 1b bound and internalized into the Daudi cells in a dose dependent manner with MFIs of 859, 509, and 397 at 10, 1 and 0.1 μg/mL respectively. The internalization of the anti-human IL-4Rα Ab1 GC conjugate was comparable to that of the unconjugated Ab1. This indicates that the anti-human IL-4Rα Ab1 GC conjugate binding and internalization function was not impacted by conjugation of the anti-human IL-4Rα Ab1 to the glucocorticoid.

TABLE 10
Internalization of exemplified anti-human IL-4Rα
Ab1 GC conjugate of Example 1b into Daudi cells

| | pHrodo gMFI | | |
|---|---|---|---|
| | 0.1 μg/mL | 1 μg/mL | 10 μg/mL |
| Control IgG4P | 129 | 160 | 436 |
| Ab1 | 402 | 514 | 902 |
| Ab1 GC conjugate | 397 | 509 | 859 |

Example 4c. Inhibition of IL-4 and IL-13 induced pSTAT6 phosphorylation in human PBMCs: Inhibition of IL-4 and IL-13 mediated IL-4R pSTAT6 phosphorylation by the exemplified anti-human IL-4Rα Ab1 GC conjugate of Example 1b and anti-human IL-4Rα Ab1 were assessed in primary B and/or T cells. Human PBMCs were isolated from human blood samples by standard Ficoll-Paque™ plus (GE HEALTHCARE) density gradient centrifugation methods. Isolated cells were resuspended at 100-300 million cells in 100 mL of complete media (RPMI-1640 with 10% FBS, 1% penicillin-streptomycin solution, from Corning®, and 1% GlutaMAX™ and 0.1% (3-mercaptoethanol from Gibco™) in a T175 flask (FALCON) and stimulated with 2 μg/mL PHA (SIGMA), 0.5 μg/mL LPS (SIGMA) and 100 ng/mL recombinant human IL-6 overnight. Cells were washed with fresh media and plated at 5×10⁴ to 2×10⁵ cells/well in 96 well round bottom plates (Corning®) in 100 μL complete media containing the exemplified antibodies at 10 μg/mL diluted down in a 4-fold dilution and 11-point titration. The cells were incubated with the samples for 30 minutes at room temperature and then stimulated with human recombinant IL-4 (20 ng/mL final concentration) or human recombinant IL-13 (100 ng/mL final concentration, R&D SYSTEMS) for 12 minutes at room temperature. Stimulation was stopped by the addition of 120 μL of 1× Lyse/Fix Buffer (BD BIOSCIENCES) for 5 minutes, the plates were then centrifuged at 2000 rpm for 2 minutes and the supernatant was aspirated. The cell pellets were resuspended in 100 μL ice-cold methanol (SIGMA) and placed on ice for 20 minutes and washed with DPBS containing 2% FBS (Corning®). The cells were resuspended in 50 μL of antibody cocktail against the following proteins: CD4, CD33, CD8, and CD3 (Thermo Fisher Scientific), phosphorylated STAT6 (Biolegend®) and CD20 (BD BIOSCIENCES) and incubated for 30 minutes at room temperature and then washed with DPBS containing 2% FBS. The cell samples were analyzed using a flow cytometer. Analysis was performed using FlowJo software and statistical analysis is performed using GraphPad Prism 9. Curves were generated by fitting a sigmoidal curve of the log (Ab concentration) vs. the percent inhibition of phosphorylated STAT6 of each individual donor cell population (n=2).

The results in Table 11A show that the exemplified anti-human IL-4Rα Ab1 GC conjugate of Example 1b inhibited IL-4 induced STAT6 phosphorylation in both CD4⁺ T cells (IC₅₀ of 0.017 μg/mL) and B cells (IC₅₀ of 0.041 μg/mL) in a dose dependent manner.

The results in Table 11B, show that the exemplified anti-human IL-4Rα Ab1 GC conjugate of Example 1b inhibited IL-13 induced STAT6 phosphorylation in B cells (IC₅₀ of 0.064 μg/mL) in a dose dependent manner. This demonstrates the ability of the human IL-4Rα Ab1 GC conjugate to block both the IL-4 and IL-13 signaling through the IL-4R.

TABLE 11A
Inhibition of IL-4 induced STAT6 phosphorylation
in human T and B cells by exemplified anti-human
IL-4Rα Ab1 GC conjugate of Example 1b

Inhibition of IL-4 induced STAT6
phosphorylation

| | CD4+ T cells | B cells |
|---|---|---|
| | IC₅₀ (μg/mL) | IC₅₀ (μg/mL) |
| Ab1 | 0.017 | 0.019 |
| Ab1 GC conjugate | 0.041 | 0.046 |

TABLE 11B
Inhibition of IL-13 induced STAT6 phosphorylation
in human B cells by exemplified anti-human
IL-4Rα Ab1 GC conjugate of Example 1b

| | Inhibition of IL-13 induced STAT6 |
|---|---|
| | phosphorylation in B cells |
| | IC₅₀ (μg/mL) |
| Ab1 | 0.030 |
| Ab1 GC conjugate | 0.064 |

Example 4d Inhibition of IL-4 induced B cell proliferation: Inhibition of B cell proliferation by the exemplified anti-human IL-4Rα Ab1 GC conjugate of Example 1b and anti-human IL-4Rα Ab1 and Ab7 was assessed in primary B cells isolated from human PBMCs. Human PBMCs were isolated from human blood samples by standard Ficoll-Paque™ plus (GE HEALTHCARE) density gradient centrifugation methods, and primary B cells were isolated from the PBMC suspension by negative selection with EasySep™ Human Naïve B cell Enrichment kit according to the manufacturer's protocol (STEMCELL™ Technologies). Isolated human primary B cells were resuspended at 1×10⁶ cells/mL and plated in polystyrene 96-well, u-bottom plates in complete medium (RPMI-1640 containing 10% Fetal bovine serum, 1×MEM-nonessential amino acids, 1 mM sodium pyruvate, 1× penicillin-streptomycin solution (all from Corning®) and 1× GlutaMAX™ (Gibco™), 0.1% (3-mercaptoethanol (LIFE TECHNOLOGIES). Cells were pretreated with anti-human IL-4Rα Ab1 GC conjugate, anti-human IL-4Rα Ab1 and Ab7, (6aR,6b S,7S,8aS,8b S,10S,11aR,12aS,12b S)-10-(3-((3-aminobenzyl)oxy)-2-fluoro-6-methylphenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (compound of Preparation 6, herein also referred to as “GC1”), or isotype control for 0.5-1 hour at 66.67 nM diluted 4-fold and 10-point titration. Cells were stimulated with Human CD40/TNFRSF5 Antibody (200 ng/mL; R&D SYSTEMS) and with IL-4 recombinant human protein (5 ng/mL; R&D SYSTEMS) for 2 days at 37° C. and 5% CO₂. Cells were then pulsed with [³H]-thymidine (1 mCi thymidine/well; PerkinElmer®) for 18 hours at 37° C. and level of [³H]-thymidine incorporation was measured by a Microplate Counter (MicroBeta 2; PerkinElmer®) and expressed as a cell count per minute (CCPM). Statistical analysis was performed using GraphPad Prism 9 and curves were generated by fitting a sigmoidal curve of the log (Ab concentration) vs. the mean percent inhibition of CCPM compared to CD40 stimulation alone from two donors.

The results in Table 12A, show that the exemplified anti-human IL-4Rα Ab1 GC conjugate of Example 1b inhibited IL-4 induced B cell proliferation (IC₅₀ of 1.41 nM), comparable to the unconjugated anti-human IL-4Rα Ab1 (IC₅₀ of 1.31 nM) in a dose-dependent manner. GC1 alone was comparable to the isotype control, i.e., no significant inhibition was observed.

Table 12B shows representative results of inhibition of IL-4 induced B cell proliferation by anti-human IL-4Rα Ab1 and Ab7.

TABLE 12A
Inhibition of IL-4 induced B cell proliferation by exemplified
anti-human IL-4Rα Ab1 GC conjugate of Example 1b

| | Inhibition of B cell | % Inhibition of B cell |
|---|---|---|
| | proliferation | proliferation |
| | IC₅₀ (nM) | at 66.67 nM |
| Ab1 | 1.31 | 89.59 |
| Ab1 GC conjugate | 1.41 | 99.02 |
| Isotype Control | NA | 10.53 |
| GC1 | NA | 25.74 |

TABLE 12B
Inhibition of IL-4 induced B cell proliferation
by exemplified anti-human IL-4Rα antibodies

| | Inhibition of B cell |
|---|---|
| | proliferation |
| | IC₅₀ (nM) |
| Ab1 | 1.32 |
| Ab7 | 0.95 |

Example 4e. glucocorticoid receptor mediated inhibition of anti-CD40 induced B cell proliferation: Inhibition of B cell proliferation by the exemplified anti-human IL-4Rα Ab1 GC conjugate of Example 1b and anti-human IL-4Rα Ab1 was assessed in primary B cells isolated from human PBMCs. Human PBMCs were isolated from human blood samples by standard Ficoll-Paque™ plus (GE HEALTHCARE) density gradient centrifugation methods, and primary B cells were isolated from the PBMC suspension by negative selection with EasySep™ Human Naïve B cell Enrichment kit according to the manufacturer's protocol (STEMCELL™ Technologies). Isolated human primary B cells were resuspended at 1×10⁶ cells/mL and plated in polystyrene 96-well, u-bottom plates in complete medium (RPMI-1640 containing 10% Fetal bovine serum, 1×MEM-nonessential amino acids, 1 mM sodium pyruvate, 1× penicillin-streptomycin solution (all from Corning®) and 1× GlutaMAX™ (Gibco™), 0.1% (3-mercaptoethanol (LIFE TECHNOLOGIES). Cells were pretreated with anti-human IL-4Rα Ab1, anti-human IL-4Rα Ab1 GC conjugate, compound of Preparation 6 (GC1) or isotype control for 0.5-1 hour at 100 nM diluted 4-fold and 9-point titration. Cells were stimulated with Human CD40/TNFRSF5 Antibody (200 ng/mL; R&D SYSTEMS) for 2 days at 37° C. and 5% CO₂. Cells were then pulsed with [³H]-thymidine (1 mCi thymidine/well; PerkinElmer®) for 18 hours at 37° C. and level of [³H]-thymidine incorporation was measured by a Microplate Counter (MicroBeta 2; PerkinElmer®) and expressed as a cell count per minute (CCPM). Statistical analysis was performed using GraphPad Prism 9 and curves were generated by fitting a sigmoidal curve of the log(Ab concentration) vs. the mean percent inhibition of CCPM compared to no stimulation from 2 donors.

The results in Table 13 and FIG. 5, show that the exemplified anti-human IL-4Rα Ab1 GC conjugate of Example 1b and GC1 alone inhibited anti-CD40 induced B cell proliferation (IC₅₀ of 5.51 nM and 3.73 nM respectively) in a dose dependent manner. Minimal inhibition was observed with the anti-human IL-4Rα Ab1 which was comparable to the isotype alone. This shows that the GC in the anti-human IL-4Rα Ab1 GC conjugate is effectively delivered into the B cells, and effectively modulates the GC receptor agonist in the B cells to inhibit B cell proliferation, independent of IL-4R mediated inhibition of B cell proliferation.

TABLE 13
Glucocorticoid agonist receptor mediated inhibition
of CD40 induced B cell proliferation by the anti-
human IL-4Rα Ab1 GC conjugate of Example 1b

| | Inhibition of B cell | Inhibition of B cell |
|---|---|---|
| | proliferation | proliferation at |
| | IC₅₀ (nM) | 100 nM % inhibition |
| Ab1 | NA | 3.30 |
| Ab1 GC conjugate | 5.51 | 66.61 |
| Isotype | NA | 3.10 |
| GC1 | 3.73 | 66.55 |

Example 4f Inhibition of IL-4 induced CD23 and Induction of CD163 expression on Myeloid cells: Inhibition of IL-4 induced CD23 expression and induction of glucocorticoid induced CD163 expression by the exemplified anti-human IL-4Rα Ab1 GC conjugate of Example 1b was assessed in myeloid cells. CD163 is expressed by monocytic cells and has been associated with autoimmune disorders. Glucocorticoids have been shown to induce CD163 expression which is thought to contribute to the anti-inflammatory effects of glucocorticoids. To determine the effect of the glucocorticoid in the anti-human IL-4Rα Ab1 GC conjugate CD163 expression levels are assessed on the surface of monocytic cells in response to treatment with the anti-human IL-4Rα Ab1 GC conjugate.

Briefly, fresh LRS-WBC donors are obtained from the San Diego Blood Bank, for PBMC isolation by standard Ficoll-Paque™ plus (GE HEALTHCARE) density gradient centrifugation methods. Cells were seeded at 2×10⁵ cells/well in a 96-well flat bottom plate. 50 μL of 3× serially diluted antibodies were added to the wells and incubated at 37° C. with 5% CO₂ for 30 minutes. Then 50 μL of 3× stimulation of recombinant human IL-4 (R&D SYSTEMS) in complete media was added to the wells to a final concentration of 10 ng/mL. The plates were incubated 37° C. with 5% CO₂ for 48 hours, cells were washed and resuspended in FACS buffer containing Human TruStain FcX™. Brilliant Violet 605™ anti-human CD163 antibody, Brilliant Violet 785™ anti-human CD33 antibody, FITC anti-human CD3 antibody (from Biolegend®), CD20 monoclonal antibody (2H7) PerCP-Cyanine5.5, and CD23 monoclonal antibody (EBVCS2), APC (from THERMO FISHER SCIENTIFIC). Cells were incubated at 4° C. for 30 minutes, washed twice with FACS buffer and resuspended in a final volume of 100 μL FACS buffer. The viability dye, Sytox™ blue (THERMO FISHER SCIENTIFIC) was added to the wells and the samples were analyzed via a flow cytometer (LSRFortessa™ X-20; BD BIOSCIENCES). Data analysis was performed using FlowJo software. Myeloid cells were identified as Sytox™ blue, CD3, and CD20 negative, CD33 positive cells. Data was presented as sigmoidal curve fits of the geometric mean fluorescent intensity (gMFI) of the myeloid cells vs. the log(Ab concentration) of 3 or 4 donors (mean±SD) and statistical analysis is performed using GraphPad Prism 9.

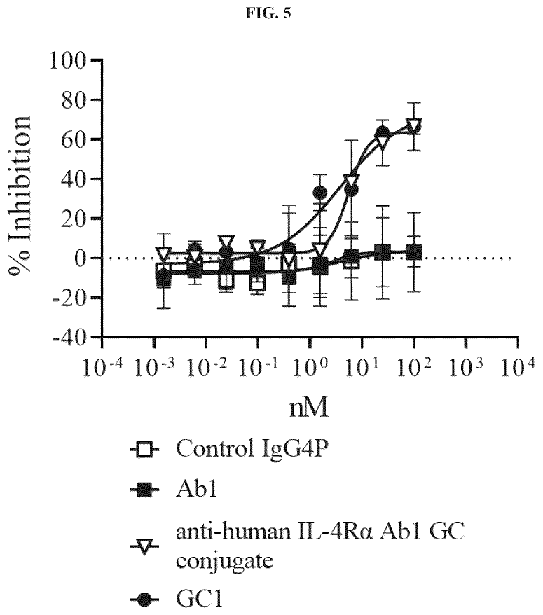

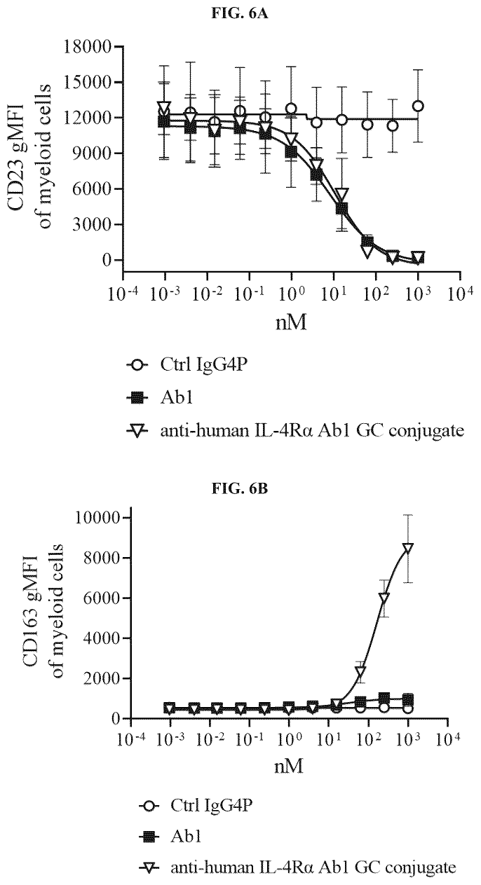

The results in Table 14 and FIGS. 6A and 6B, show that the exemplified anti-human IL-4Rα Ab1 GC conjugate of Example 1b and the anti-human IL-4Rα Ab1 inhibited IL-4 (FIG. 6A) induced CD23 expression on myeloid cells with IC₅₀ of 10.75 nM and 8.27 nM, respectively. Furthermore, the results showed that the anti-human IL-4Rα Ab1 GC conjugate significantly increased CD163 expression at 1000 nM, 250 nM, and 63 nM (gMFI of 8462, 5984, and 2317, respectively) compared to unconjugated Ab1 at 1000 nM (gMFI of 941) (FIG. 6B). This shows that the exemplified anti-human IL-4Rα Ab1 GC conjugate can both, inhibit IL4R mediated responses mediated by the Ab1 and induce GC receptor mediated responses within the same cells, demonstrating the dual functionality of the anti-human IL-4Rα Ab1 GC conjugate from the antibody and the glucocorticoid, and effective delivery of the GC into the cell by the antibody.

TABLE 14
Inhibition of IL-4 induced CD23 expression and GC-
induced CD163 expression in myeloid cells by the
anti-human IL-4Rα Ab1 GC conjugate of Example 1b

| | Inhibition of IL-4 induced | Induction of CD163 |
|---|---|---|
| | CD23 Expression | Expression at |
| | IC₅₀ (nM) | 1000 nM gMFI |
| Ab1 | 8.27 | 941 |
| Ab1 GC conjugate | 10.75 | 8462 |

Example 4g. Induction of glucocorticoid induced gene expression in Th2 differentiated T cells: The induction and expression of three glucocorticoid receptor-mediated genes (Tsc22d3, Fkbp5, and Zbtb16) and one cytokine are measured in primary human T cells that are differentiated ex vivo to a Th2 phenotype representative of cells involved in type 2 inflammation and disease.

Human Th2 cells were differentiated in vitro, by culturing purified naïve CD4 T cells with anti-human CD3 (BioXCell #BE0001-2), anti-human CD28 (BioLegend #302934), anti-human IFNγ (R&D Systems #MAB285-500), recombinant human IL-2 (R&D Systems #202-IL-050/CF), and recombinant human IL-4 (R&D Systems #6507-IL-100/CF), for 14 days. Terminally differentiated Th2 cells were then rested without IL-4 for 12 hours prior to the assay, to return IL-4R surface expression. Flow cytometry staining was used to assess cell purity on a BD LSRFortessa Cell Analyzer. Th2 cells were confirmed CD4+(anti-human CD4-eFluor-450, Fisher Scientific #48-0047-42), GATA3+(anti-human GATA3-PerCP/Cyanine5.5, BioLegend #653812), and IL4R+(anti-IL-4Rα antibody-Alexa Fluor-647, Lilly). 1×10⁶ Th2 cells/well were treated with 100 nM IL4R-GC Ab1 and stimulated with Human T-Activator CD3/CD28 Dynabeads (Fisher Scientific #11132D) for 24 hrs, at 37° C. Cells from each assay condition were lysed in RLT buffer (Qiagen #79216) & frozen at −80° C. RNA was then isolated using the Rneasy 96 Kit (Qiagen #74181) according to the manufacturer protocol. The data is representative of four technical replicates, per assay condition (n=4).

Gene expression levels were determined using the NanoString platform with a custom gene panel (Table 15) following manufacturer recommended protocols. RNA isolated from in-vitro cultures was quantified using OD₂₆₀ measurements on the Cytation 5 (Biotek) platform. RNA was diluted to 20 ng/μl in Rnase free water, and a total of 100 ng (in 5 μl) was used to prepare the NanoString cartridges. Raw mRNA counts were normalized using the nSolver Advanced Analysis software (Version 2.0.115) following manufacturer recommended data processing methods. All mRNA counts were initially scaled to intra-sample binding density controls, then experimental genes were further normalized to a housekeeping gene index following dynamic housekeeping gene selection for the entire data set, minimizing housekeeper variance. Normalized gene counts were transformed to a Log 2 scale, and relative expression of experimental treatment groups was determined by subtracting the average Log 2 expression of the “No Treatment” group. Statistical analysis is performed using GraphPad Prism 9. Results are reported as mean log 2 expression normalized to the mean of the replicates of the no treatment group for each replicate, for each gene within the treatment group+SD (n=4 for all groups). Differences are assessed using two-way analysis of variance (ANOVA) on the normalized values and comparisons against the no treatment group are evaluated using Bonferroni correction for multiple comparisons, with a significance level of p<0.001.

The results in Table 16, show that the anti-human IL-4Rα Ab1 GC conjugate of Example 1b induced mRNA expression of glucocorticoid receptor mediated genes Tsc22d3 (elevated 2.96 fold, p-value<0.001), Fkbp5 (elevated 1.81 fold, p-value<0.001), Zbtb16 (elevated 1.29 fold, p-value<0.003), and reduced mRNA expression of the cytokine IL-5 by 2.29 fold, p-value<0.0001, in Th2 differentiated T cells. This shows that the anti-human IL4Rα Ab1 GC conjugate can induce GC/GR mediated gene modulation and reduce IL-5 expression within cells representative of cells involved in type 2 inflammation and disease.

TABLE 15
Gene panel tested for GC/GR gene modulation by the
anti-human IL4Rα Ab1 GC conjugate of Example 1b

| | Accession | |
|---|---|---|
| Gene Name | Number | Designation |
| Tsc22d3 Hs | NM_004089.3 | Endogenous |
| Fkbp5 Hs | NM_001145775.1 | Endogenous |
| Zbtb 16 Hs | NM_006006.6 | Endogenous |
| IL5 | NM_000879.2 | Endogenous |
| Rp14 Hs | NM_000968.4 | Housekeeping |
| TBP Hs | NM_003194.4 | Housekeeping |
| UBB Hs | NM_001281718.1 | Housekeeping |
| Gapdh Hs | NM_001256799.1 | Housekeeping |
| Tubb4a Hs | NM_006087.2 | Housekeeping |
| G6PD Hs | NM_001042351.2 | Housekeeping |
| Gusb Hs | NM_000181.3 | Housekeeping |
| ABCF1 Hs | NM_001025091.1 | Housekeeping |
| OAZ1 Hs | NM_004152.2 | Housekeeping |
| POLR2A Hs | NM_000937.4 | Housekeeping |
| BABAM1 Hs | NM_014173.4 | Housekeeping |
| Hprt1 Hs | NM_000194.2 | Housekeeping |

TABLE 16
GC/GR gene expression modulation of Th2 differentiated T cells
by the anti-human IL-4Rα Ab1 GC conjugate of Example 1b

| | Mean Difference | | Adjusted P |
|---|---|---|---|
| Gene | (log2) | 95% C.I. | Values |
| Tsc22d3 | 2.959 | 2.226 to 3.692 | <0.0001 |
| Fkbp5 | 1.807 | 1.074 to 2.541 | <0.0001 |
| Zbtb16 | 1.29 | 0.5571 to 2.023 | 0.0003 |
| IL-5 | −2.289 | −3.023 to −1.556 | <0.0001 |

Example 4h. Inhibition of Cytokine secretion from Human PBMCs: PBMCs were isolated from Ficoll-layered blood centrifuged at 400×g for 30 minutes at room temperature. The PBMC layer was washed with sterile RPMI-1640 media (Corning® Cat. #10041CV) and centrifuged at 1500 rpm for 5 min, at 4° C. Red blood cells are lysed by resuspending the cell pellet in 5 mL of ACK Lysing Buffer (GIBCO Cat. #A1049201) and incubating at room temperature for 5 minutes. After RBC lysis cells are washed and resuspended at 1×10⁶ cells/mL in warmed complete media (RPMI-1640 with 10% FBS, 1% MEM nonessential amino acid solution, 1% penicillin-streptomycin solution, from Corning®, and 1% GlutaMAX™ and 0.1% (3-mercaptoethanol from Gibco™). Cells are seeded at 1×10⁶ cells/well in a 96-well flat bottom plate. 50 μL of 4× control IgG, anti-human IL4Rα Ab1, anti-human IL4Rα Ab1 GC conjugate of Example 1b, or GC1 alone (final concentration of 100 nM serially diluted by 3 for a 9-point curve) are added to the wells. Then 50 μL of 4× stimulation cocktail consisting of 40 μg/mL PHA-M (phytohemagglutinin, M form; LIFE TECHNOLOGIES) and 400 ng/mL PMA (phorbol 12-myristate 13-acetate; TOCRIS) in complete media is added to the wells. The plates are incubated at 37° C. with 5% CO₂ for 48 hours. After the incubation period, plates are centrifuged at 2000 rpm for 1 min and the top 100 μL of the supernatants are removed and stored at −80° C., until ready to perform cytokine detection. Cytokine release is detected using the U-PLEX Custom Biomarker [Human] Multiplex Assay (MESO SCALE DISCOVERY), according to the manufacturer's protocol. Statistical analysis is performed using GraphPad Prism 9. Data was presented as sigmoidal curve fits of the mean of the individual donors' percent inhibition for each cytokine (n=2) vs. the log(Ab concentration) and statistical analysis is performed using GraphPad Prism 9.

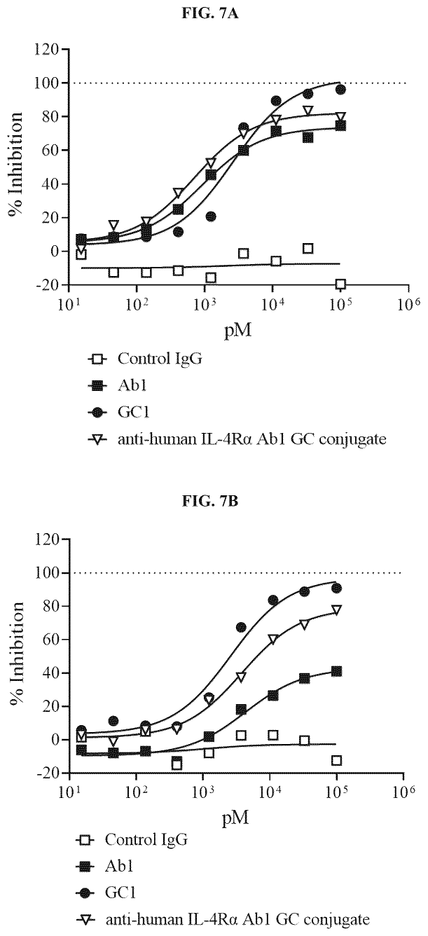

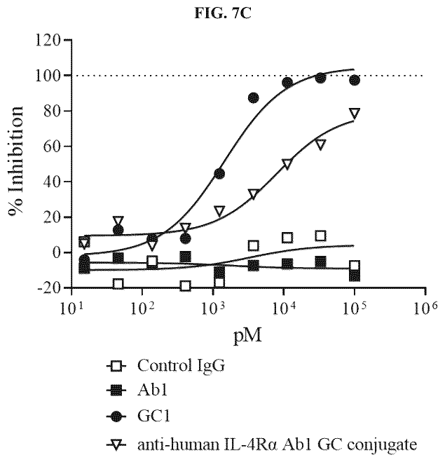

The results in Table 17 and FIGS. 7A-C, show that the anti-human IL-4Rα Ab1 GC conjugate of Example 1b significantly inhibited both IL-4R mediated and glucocorticoid receptor mediated cytokine secretion. The anti-human IL-4Rα Ab1 GC conjugate inhibited MDC secretion similarly to the anti-human IL-4Rα Ab1 (79.7% and 74.7% at 100 nM, respectively). The anti-human IL-4Rα Ab1 GC conjugate inhibited GM-CSF secretion more robustly than Ab1 alone (77.6% and 40.9% at 100 nM, respectively, FIG. 4B). Furthermore, the anti-human IL-4Rα Ab1 GC conjugate and GC1 payload inhibited IL-5 secretion by 78.5% and 97.4% at 100 nM, respectively (FIG. 4C), whereas Ab1 had no effect (−13.3% at 100 nM). This data demonstrates the dual functionality of the anti-human IL-4Rα Ab1 GC conjugate in inhibiting both IL-4R-dependent and IL4R-independent cytokine secretion. This demonstrates additional functionality and improved efficacy of the anti-human IL-4Rα Ab1 GC conjugate in cytokine inhibition when compared to the antibody alone.

TABLE 17
Inhibition of IL-4R mediated and GC mediated cytokine secretion
by the anti-human IL-4Rα Ab1 GC conjugate of Example 1b

| | % Inhibition of | % Inhibition of | % Inhibition of |
|---|---|---|---|
| | MDC secretion | GM-CSF secretion | IL-5 secretion |
| | at 100 nM | at 100 nM | at 100 nM |
| Ab1 | 74.7 | 40.9 | −13.3 |
| Ab1 GC | 79.7 | 77.6 | 78.5 |
| conjugate | | | |
| Isotype | −19.5 | −12.6 | −7.4 |
| GC1 | 96.1 | 90.8 | 97.4 |

### Example 5. Effector Function and Fcγ Receptor Binding of the Exemplified Anti-Human IL-4Rα Antibody GC Conjugate of Example 1b

Example 5a. Human Fcγ receptor binding. The binding affinity of the exemplified anti-IL-4Rα GC conjugate of Example 1b and anti-human IL-4Rα antibodies to human Fcγ receptors was evaluated by surface plasmon resonance (SPR) analysis. A series S CM5 chip (Cytiva P/N BR100530) was prepared using the manufacturer's EDC/NHS amine coupling method (Cytiva P/N BR100050). Briefly, the surfaces of all 4 flow cells (FC) were activated by injecting a 1:1 mixture of EDC/NHS for 7 minutes at 10 μL/minute. Protein A (Calbiochem P/N 539202) was diluted to 10011 g/mL in 10 mM acetate, pH 4.5 buffer, and immobilized for approximately 4000 RU onto all 4 FCs by 7 minute injection at a flow rate of 10 μL/minute. Unreacted sites were blocked with a 7 minute injection of ethanolamine at 10 μL/minute. Injections of 2×10 μL of glycine, pH 1.5, was used to remove any noncovalently associated protein. Running buffer was 1×HBS-EP+(TEKNOVA, P/N H8022). The FcγR extracellular domains (ECDs)-FcγRI (CD64), FcγRIIA_131R, and FcγRIIA_131H (CD32a), FcγRIIIA_158V, FcγRIIIA_158F (CD16a), and FcγRIIb (CD32b) were produced from stable CHO cell expression and purified using IgG Sepharose and size exclusion chromatography. For FcγRI binding, antibodies were diluted to 2.511 g/mL in running buffer, and approximately 150 RU of each antibody was captured in FCs 2 through 4 (RU captured). FC1 was the reference FC, therefore no antibody was captured in FC1. FcγRI ECD was diluted to 200 nM in running buffer and then two-fold serially diluted in running buffer to 0.78 nM. Duplicate injections of each concentration were injected over all FCs at 40 μL/minute for 120 seconds followed by a 1200 second dissociation phase. Regeneration was performed by injecting 15 μL of 10 mM glycine, pH 1.5, at 30 μL/minute over all FCs. Reference-subtracted data was collected as FC2 FC1, FC3-FC1, and FC4-FC1 and the measurements were obtained at 25° C. The affinity (K_{D}) was calculated using either steady state equilibrium analysis with the Scrubber 2 Biacore Evaluation Software or a “1:1 (Langmuir) binding” model in BIA Evaluation. For FcγRIIa, FcγRIIb, and FcγRIIIa binding, antibodies were diluted to 5 μg/mL in running buffer, and approximately 500 RU of each antibody was captured in FCs 2 through 4). FC1 was the reference FC. Fcγ receptor ECDs were diluted to 101.1M in running buffer and then serially diluted 2-fold in running buffer to 39 nM. Duplicate injections of each concentration were injected over all FCs at 40 μL/minute for 60 seconds followed by a 120 second dissociation phase. Regeneration was performed by injecting 15 μL of 10 mM glycine, pH 1.5, at 30 μL/minute over all FCs. Reference-subtracted data was collected as FC2-FC1, FC3-FC1, and FC4-FC1, and the measurements were obtained at 25° C. The affinity (K_{D}) was calculated using the steady state equilibrium analysis with the Scrubber 2 Biacore Evaluation Software. Each receptor was assayed at least two times.

The results in Table 18 Å show the binding affinities (K_{D}) of the exemplified anti-human IL-4Rα Ab1 GC conjugate of Example 1b to human FcγRI, FcγRIIa, FcγRIIb, and FcγRIIIa receptor ECDs.

The results in Table 18B, summarize the binding affinities (K_{D}) of the exemplified anti-human IL-4Rα antibodies Ab1 and Ab7 to human FcγRI, FcγRIIa, FcγRIIb, and FcγRIIIa receptor ECDs.

TABLE 18A
Binding affinities of exemplified anti-human IL-4Rα Ab1
GC conjugate of Example 1b to human Fcγ receptors

| | | | | anti-human IL-4Rα | | |
|---|---|---|---|---|---|---|
| | IgG1 control | | | Ab1 GC conjugate | | |
| Fcγ Receptor | Average K_{D} | | Std Dev | Average K_{D} | | Std Dev |
| FcγRI | 55.9 | pM | 7.6 | 640.5 | pM | 88.2 |
| FcγRIIA_131H | 0.69 | μM | 0.03 | 3.65 | μM | 0.12 |
| FcγRIIA_131R | 0.76 | μM | 0.03 | 1.82 | μM | 0.03 |
| FcγRIIb | 3.75 | μM | 0.35 | 1.96 | μM | 0.13 |
| FcγRIIIA_158V | 0.18 | μM | 0.01 | 4.92 | μM | 0.12 |
| FcγRIIIA_158F | 0.91 | μM | 0.08 | >10 | μM | |

TABLE 18B
Binding affinities of exemplified anti-human IL-4Rα antibodies to human Fcγ receptors

| | Hu IgG1 | | | Hu IgG4 SP | | | | | | | | |
|---|---|---|---|---|---|---|---|---|---|---|---|---|
| | control | | | control | | | Ab1 | | | Ab7 | | |
| | Average | | Std | Average | | Std | Average | | Std | Average | | Std |
| Fcγ Receptor | K_{D} | | Dev | K_{D} | | Dev | K_{D} | | Dev | K_{D} | | Dev |
| FcγRI | 52.1 | pM | 2.1 | 418.7 | pM | 16.5 | 442.3 | pM | 21.4 | 42.8 | pM | 3.9 |
| FcγRIIA_131H | 0.68 | μM | 0 | 5.31 | μM | 0.03 | 3.75 | μM | 0.11 | 1.24 | μM | 0.01 |
| FcγRIIA_131R | 0.74 | μM | 0 | 2.31 | μM | 0.07 | 1.67 | μM | 0.06 | 0.78 | μM | 0.03 |
| FcγRIIb | 3.11 | μM | 0.1 | 2.78 | μM | 0.42 | 2.05 | μM | 0.2 | 3.02 | μM | 0.34 |
| FcγRIIIA_158V | 0.20 | μM | 0.01 | 7.35 | μM | 0.84 | 6.22 | μM | 0.8 | 0.44 | μM | 0.01 |
| FcγRIIIA_158F | 1.29 | μM | 0.04 | >10 | μM | | >10 | μM | | 2.63 | μM | 0.18 |

Example 5b. Antibody dependent cellular cytotoxicity (ADCC): In vitro ADCC assays of the exemplified anti-human IL-4Rα Ab1 GC conjugate of Example 1b was evaluated with reporter gene based ADCC assay. Briefly, Daudi cells (ATCC, #CCL-213) expressing human IL-4Rα and human CD20 as the target cell line and Jurkat cells expressing functional FcγRIIIa (V158)-NFAT-Luc (Eli Lilly and Company) as the effector cell line were used. All test molecules and cells were diluted in assay medium containing RPMI-1640 (no phenol red) with 0.1 mM non-essential amino acids (NEAA), 1 mM sodium pyruvate, 2 mM L-glutamine, 500 U/mL of penicillin-streptomycin, and 0.1% w/v BSA. Test antibodies were first diluted to a 3× concentration of 30 nM and then serially diluted 7 times in a 1:4 ratio. 50 μL/well of each molecule was aliquoted in triplicate in white opaque bottom 96-well plate (Costar, #3917). CD20 antibody was used as a positive control. Daudi target cells were then added to the plates at 5×10⁴ cells/well in 50 aliquots, and incubated for 1 hour at 37° C. Next, Jurkat V158 cells were added to the wells at 1.5×10⁵ cells/well in 50 μL aliquots and incubated for 4 hours at 37° C., followed by addition of 100 μL/well of One-Glo Luciferase substrate (Promega, #E8130). The contents of the plates were mixed using a plate shaker at low speed, incubated at room temperature for 5 minutes, and the luminescence signal was read on a BioTek microplate reader (BioTek Instruments) using 0.2 cps integration. Data was analyzed using GraphPad Prism 9 and the relative luminescence units (RLU) for each antibody concentration were plotted in a scatter format of antibody concentration versus RLU. Results were representative of two independent experiments.

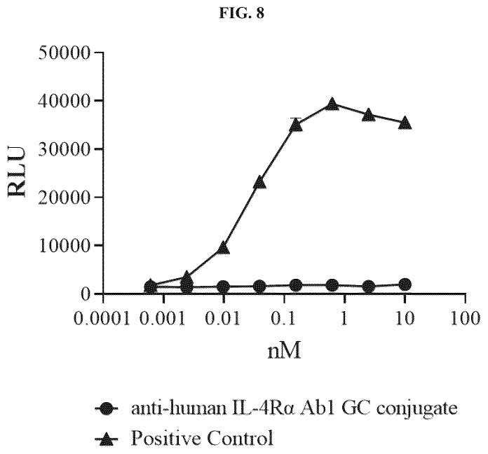

The results in FIG. 8, show that the exemplified anti-human IL-4Rα Ab1 GC conjugate of Example 1b lacked or had no ADCC activity, when compared to the positive control. Similarly, representative results (not shown) for exemplified anti-human IL-4Rα Ab1, Ab4, and Ab7 showed that the antibodies lacked or had no significant ADCC activity, when compared to the positive control.

Example 5c. Complement dependent cellular cytotoxicity (CDC): In vitro CDC assays of the exemplified antibodies was conducted using Daudi cells (ATCC, #CCL-213). All test antibodies, complement, and cells were diluted in assay medium consisting of RPMI-1640 (no phenol red) with 0.1 mM non-essential amino acids (NEAA), 1 mM sodium pyruvate, 2 mM L-glutamine, 500 U/mL of penicillin-streptomycin, and 0.1% w/v BSA. Test antibodies were first diluted to a 3× concentration of 600 nM and then serially diluted 7 times in a 1:4 ratio. 50 μL/well of each antibody (including the CD20 positive control antibody) was aliquoted in triplicate in white opaque bottom 96-well plate (Costar, #3917). Daudi target cells were added at 5×10⁴ cells/well at 50 μL/well and incubated for 1 hour at 37° C. Next, human serum complement (Quidel, #A113) quickly thawed in a 37° C. water bath was diluted 1:6 in assay medium and added at 50 μL/well to the assay plate. The plate was incubated for 2 hours at 37° C., followed by addition of 100 μL/well CellTiter Glo substrate (Promega, #G7571). The contents of the plates were mixed using a plate shaker at low speed, incubated at room temperature for 5 minutes, and the luminescence signal was read on a BioTek microplate reader (BioTek instruments) using 0.2 cps integration. Data was analyzed using GraphPad Prism 9 and the relative luminescence units (RLU) for each antibody concentration were plotted in a scatter format of antibody concentration versus RLU. Results are representative of two independent experiments.

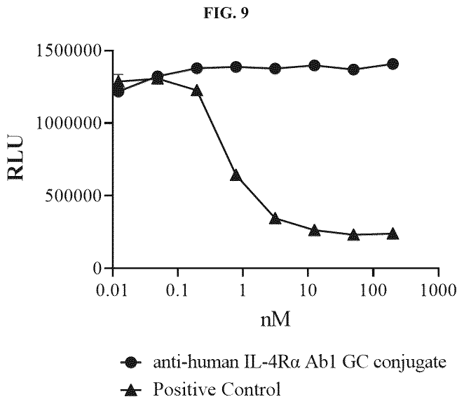

The results in FIG. 9, show that the exemplified IL-4Rα Ab GC conjugate of Example 1b does not elicit a CDC response compared to the positive control. Similarly, representative results (not shown) for exemplified anti-human IL-4Rα Ab1, Ab4, and Ab7 showed the antibodies did not elicit a CDC response when compared to the positive control.

### Example 6. Biophysical Properties of the Anti-Human IL-4Rα Ab1 GC Conjugate of Example 1b

Biophysical properties of the exemplified anti human IL-4Rα Ab1 GC conjugate of Example 1b was evaluated for developability.

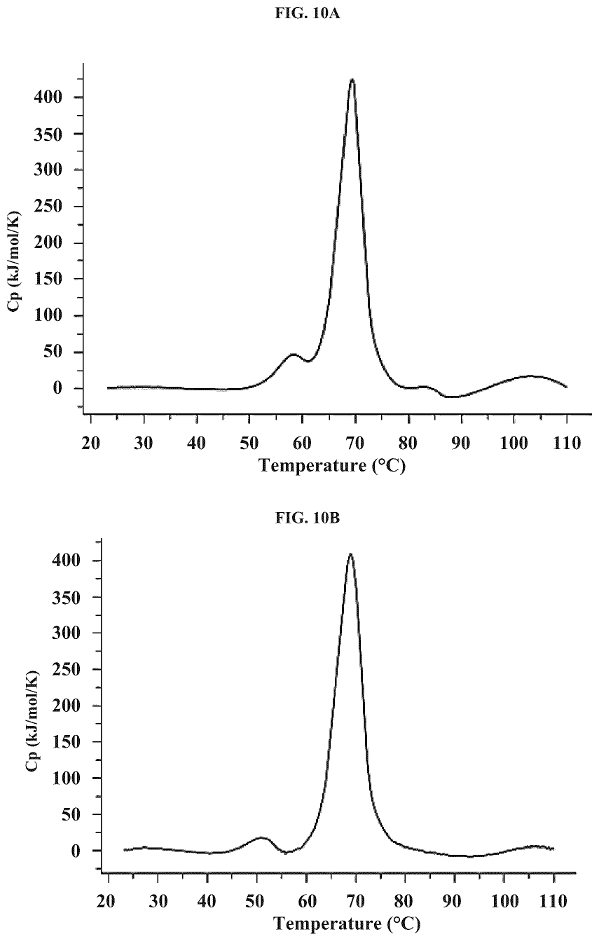

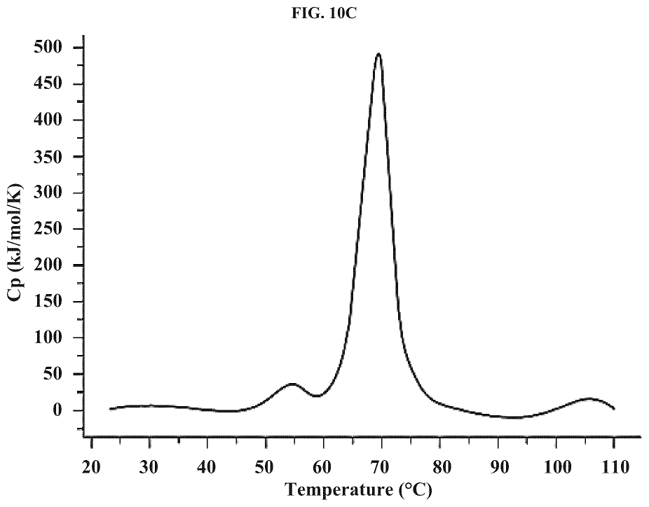

Example 6a. Thermal stability: Differential Scanning calorimetry (DSC) was used to evaluate the stability of the exemplified antibodies against thermal denaturation. The onset of melting (Tonset) and thermal melting temperatures (TM1 and TM2) of the anti-human IL-4Rα Ab1 GC conjugate in PBS, pH 7.2 buffer, Acetate, pH5 and Histidine, pH6 were obtained (Table 19). Although the thermal transition temperatures for each domain were not well resolved, the data in Table 19 and FIGS. 10A-C, show that the conjugation of the exemplified linker-payload to the 4 engineered cysteines on the exemplified resulted in acceptable thermal stability.
Example 6b. Aggregation upon temperature stress: The solution stability of the exemplified anti-human IL-4Rα Ab1 GC conjugate over time is assessed at approximately 100 mg/mL in a common 5 mM histidine pH 6.0 buffer with excipients. Concentrated samples were incubated for a period of 4 weeks at 5° C. and 35° C., respectively. Following incubation, samples were analyzed for the percentage of high molecular weight (% HMW) species using size exclusion chromatography (SEC). The results in Table 20, show that conjugation of the exemplified linker-payload to the 4 engineered cysteines on the Ab1 results in an acceptable level of aggregation for the anti-human IL-4Rα Ab1 GC conjugate over a 4-week time period at 35° C.

TABLE 19
Thermal stability (° C.) of the exemplified anti-
human IL-4Rα Ab1 GC conjugate of Example 1b

| | Buffer | Tonset | TM1 | TM2 |
|---|---|---|---|---|
| Ab1 GC | PBS pH 7.2 | 52.4 | 57.9 | 69.6 |
| conjugate | Acetate pH 5.0 | 47.9 | 50.5 | 69.4 |
| | Histidine pH 6.0 | 49.4 | 54.2 | 69.6 |

TABLE 20
Biophysical properties of exemplified anti-
human IL-4Rα Ab1 GC conjugate of Example 1b

| | | anti-human IL-4Rα | |
|---|---|---|---|
| | | Ab1 GC conjugate | |
| concentration | Buffer | 100 mg/mL | 150 mg/mL |
| % HMW after 4-week | Histidine pH 6.0 | 2.2 | 2.3 |
| incubation at 5° C. | | | |
| % HMW change after | Histidine pH 6.0 | 3.7 | 4.3 |
| 4-week incubation | | | |
| at 35° C. | | | |

### Example 7. In Vivo Function of the Anti-Human IL-4Rα Ab1 GC Conjugate of Example 1b

In vivo efficacy in a type IV hypersensitivity of a fully humanized mouse model: A humanized mouse model of contact hypersensitivity was used to determine in vivo activity of the anti-human IL-4Rα Ab1 GC conjugate anti-human IL-4Rα Ab1.

Immunodeficient NOG mice expressing human GM-CSF and human IL-3 to support myeloid lineage development (huNOG-EXL, Taconic) were engrafted at 6 weeks of age with human CD34+ hematopoietic stem cells isolated from human cord blood. 20-24 weeks after stem cell administration the mice were assessed for sufficient human CD45 engraftment (>25% in blood) and subjected to an oxazolone-induced contact hypersensitivity protocol. On day 0, mice grouped by body weight were dosed at 10 mg/kg subcutaneously (SC) with either anti-human IL-4Rα Ab1 GC conjugate (n=8), anti-human IL-4Rα Ab1 (n=8), or a control human IgG4P antibody (n=8). The GC alone (n=6) was dosed at 0.15 mg/kg SC 24 hours prior to sensitization and each challenge. On day 1, mice were anesthetized with 5% isoflurane, their abdomens shaved, and 100 μL of 3% oxazolone in ethanol was applied to the shaved area. Mice were dosed again on day 6 at the same doses, anesthetized, and then challenged with 2% oxazolone in ethanol on both ears (10 μL/side/ear) 24 hours post dose. The dose challenge paradigm was repeated weekly for a total of 3 challenges. The inflammatory response was determined by the difference in ear thickness prior to and 24 hours following each challenge using a Miltenyi Biotec electronic caliper. P-values between groups were calculated by one-way ANOVA followed by Tukey's post hoc test and considered significant if <0.05 (GraphPad Prism).

The results in Table 21 show that the anti-human IL-4Rα Ab1 GC conjugate Example 1b was able to significantly inhibit the in vivo inflammatory responses to hapten induced contact hypersensitivity compared to all other treatments following challenges 2 and 3. Compared to isotype control, IL-4Rα Ab1 GC conjugate reduced inflammation by 80% and 78% for challenges 2 and 3, respectively. Additionally, there were trends towards activity with the unconjugated Ab1, though Ab1 only significantly inhibited inflammation following challenge 2 compared to isotype (40% reduction). Furthermore, the GC treatment using an equivalent dose and paradigm as the Ab1 GC conjugate was ineffective in the model indicating that the in vivo activity was attributed to the Ab1 GC conjugate. These results show that the anti-human IL-4Rα Ab1 GC conjugate effectively delivered the glucocorticoid to the inflamed tissue and significantly abrogated the biological effects associated with a type IV hypersensitivity reaction in a humanized mouse model, indicating that this anti-inflammatory response could be elicited in a human subject.

TABLE 21
In vivo efficacy of the anti-human IL-4Rα Ab1 GC conjugate
of Example 1b in a type IV hypersensitivity humanized mouse model

| | Challenge 1 | | Challenge 2 | | Challenge 3 | |
|---|---|---|---|---|---|---|
| | Δ Ear thickness | | Δ Ear thickness | | Δ Ear thickness | |
| | (mm) | | (mm) | | (mm) | |
| | Mean | ±SEM | Mean | ±SEM | Mean | ±SEM |
| hIgG4P Isotype | 0.033 | 0.006 | 0.090 | 0.012 | 0.115 | 0.015 |
| Control | | | | | | |
| Ab1 | 0.029 | 0.004 | 0.054* | 0.005 | 0.086 | 0.010 |
| Ab1 GC | 0.017 | 0.003 | 0.018\hat{ } | 0.003 | 0.025\hat{ } | 0.005 |
| conjugate | | | | | | |
| GC alone | 0.021 | 0.005 | 0.074 | 0.006 | 0.102 | 0.011 |

*p < 0.05 vs Isotype; ANOVA Tukey;
\hat{ }p < 0.01 vs all; ANOVA Tukey

SEQUENCE LISTING
Ab1
SEQ ID NO: 1 HCDR1 (North) for Ab1, Ab2, Ab3, Ab4, Ab5, Ab6, Ab7, and Ab8
VASGFTFSHSSMN
SEQ ID NO: 2 HCDR2 (North) for Ab1, Ab2, Ab3, Ab4, Ab5, Ab6, Ab7, Ab8, and
Ab9
YISRATGAVY
SEQ ID NO: 3 HCDR3 (North) for Ab1, Ab2, Ab3, Ab4, Ab5, Ab6, Ab7, Ab8, Ab9,
and Ab10
AREPVFDY
SEQ ID NO: 4 LCDR1 (North) for Ab1, Ab2, Ab3, Ab4, Ab5, Ab6, Ab7, and Ab8
RASQDISNYLA
SEQ ID NO: 5 LCDR2 (North) for Ab1, Ab2, Ab3, Ab4, Ab5, Ab6, Ab7, Ab8, Ab9,
and Ab10
YAASSLQS
SEQ ID NO: 6 LCDR3 (North) for Ab1, Ab2, Ab3, Ab4, Ab5, Ab6, Ab7, Ab8, and
Ab9
LQWSSYPRT
SEQ ID NO: 7 VH for Ab1, Ab2, Ab3, Ab4, Ab5, Ab6, Ab7, and Ab8
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSS
SEQ ID NO: 8 VL for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIK
SEQ ID NO: 9 HC for Ab1
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSSASTKGPCVFPLAPCSRSTSESTAALGCLVKDYFPEPVTVSWNSGALTS
GVHTFPAVLQSSGLYSLSSVVTVPSSSLGTKTYTCNVDHKPSNTKVDKRVESKYG
PPCPPCPAPEFLGGPSVFLFPPKPKDTLMISRTPEVTCVVVDVSQEDPEVKFNWYV
DGVEVHNAKTKPREEQFNSTYRVVSVLTVLHQDWLNGKEYKCKVSNKGLPSSIE
KTISKAKGQPREPQVYTLPPSREEMTKNQVSLTCLVKGFYPSDICVEWESNGQPE
NNYKTTPPVLDSDGSFFLYSRLTVDKSRWQQGNVFSCSVMHEALHNHYTQKSLS
LSLG
SEQ ID NO: 10 LC for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 11 HC DNA for Ab1
CAGGTACAGCTGGTGGAGTCTGGGGGAGGCTTGGTACAGCCTGGGGGGTCCC
TGAGACTCTCCTGTGTCGCCTCTGGATTCACCTTCAGTCATTCTAGCATGAACT
GGGTCCGCCAGGCTCCAGGGAAGGGGCTGGAGTGGGTTTCATACATTAGTCG
TGCTACTGGTGCCGTCTACTACGCAGACTCTGTAAAGGGCCGATTCACCATCT
CCAGAGATAATGCCAAAAACTCACTGTATCTGCAAATGAACAGCCTGAGAGA
CGAGGACACGGCTGTGTATTACTGTGCGAGAGAGCCGGTTTTTGACTACTGG
GGCCAGGGAACCCTGGTCACCGTCTCCTCAGCCTCCACCAAGGGCCCATGCG
TCTTCCCGCTAGCGCCCTGCTCCAGGAGCACCTCCGAGAGCACAGCCGCCCTG
GGCTGCCTGGTCAAGGACTACTTCCCCGAACCGGTGACGGTGTCGTGGAACT
CAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCAC
GAAGACCTACACCTGCAACGTAGATCACAAGCCCAGCAACACCAAGGTGGAC
AAGAGAGTTGAGTCCAAATATGGTCCCCCATGCCCACCCTGCCCAGCACCTG
AGTTCCTGGGGGGACCATCAGTCTTCCTGTTCCCCCCAAAACCCAAGGACACT
CTCATGATCTCCCGGACCCCTGAGGTCACGTGCGTGGTGGTGGACGTGAGCC
AGGAAGACCCCGAGGTCAAGTTCAACTGGTACGTGGATGGCGTGGAGGTGCA
TAATGCCAAGACAAAGCCGCGGGAGGAGCAGTTCAACAGCACGTACCGTGTG
GTCAGCGTCCTCACCGTCCTGCACCAGGACTGGCTGAACGGCAAGGAGTACA
AGTGCAAGGTCTCCAACAAAGGCCTCCCGTCCTCCATCGAGAAAACCATCTC
CAAAGCCAAAGGGCAGCCCCGAGAGCCACAGGTGTACACCCTGCCCCCATCC
CGAGAGGAGATGACCAAGAACCAGGTCAGCCTGACCTGCCTGGTCAAAGGCT
TCTACCCCAGCGACATCTGCGTGGAGTGGGAAAGCAATGGGCAGCCGGAGAA
CAACTACAAGACCACGCCTCCCGTGCTGGACTCCGACGGCTCCTTCTTCCTCT
ACAGCAGGCTAACCGTGGACAAGAGCAGGTGGCAGCAGGGGAATGTCTTCTC
ATGCTCCGTGATGCATGAGGCTCTGCACAACCACTACACACAGAAGAGCCTC
TCCCTGTCTCTGGGT
SEQ ID NO: 12 LC DNA for Ab1, Ab2, Ab3, Ab4, Ab5, Ab6, Ab7, and Ab8
GACATCCAGATGACCCAGTCTCCATCTGCCATGTCTGCATCTGTGGGAGACAG
AGTCACCATCACTTGTCGGGCGAGTCAGGACATTAGCAATTATTTAGCCTGGT
TTCAGCAGAAACCAGGGAAAGTCCCTACGCGCCTGATCTATGCTGCATCCAG
TTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTATTACTG
TCTACAGTGGTCCAGTTACCCTCGGACGTTCGGCCAAGGGACCAAGGTGGAA
ATCAAACGGACCGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
Ab2
SEQ ID NO: 1 HCDR1 (North) for Ab1, Ab2, Ab3, Ab4, Ab5, Ab6, Ab7, and Ab8
VASGFTFSHSSMN
SEQ ID NO: 2 HCDR2 (North) for Ab1, Ab2, Ab3, Ab4, Ab5, Ab6, Ab7, Ab8, and
Ab9
YISRATGAVY
SEQ ID NO: 3 HCDR3 (North) for Ab1, Ab2, Ab3, Ab4, Ab5, Ab6, Ab7, Ab8, Ab9,
and Ab10
AREPVFDY
SEQ ID NO: 4 LCDR1 (North) for Ab1, Ab2, Ab3, Ab4, Ab5, Ab6, Ab7, and Ab8
RASQDISNYLA
SEQ ID NO: 5 LCDR2 (North) for Ab1, Ab2, Ab3, Ab4, Ab5, Ab6, Ab7, Ab8, Ab9,
and Ab10
YAASSLQS
SEQ ID NO: 6 LCDR3 (North) for Ab1, Ab2, Ab3, Ab4, Ab5, Ab6, Ab7, Ab8, and
Ab9
LQWSSYPRT
SEQ ID NO: 7 VH for Ab1, Ab2, Ab3, Ab4, Ab5, Ab6, Ab7, and Ab8
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSS
SEQ ID NO: 8 VL for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIK
SEQ ID NO: 50 HC for Ab2
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSSASTKGPCVFPLAPCSRSTSGSTAALGCLVKDYFPEPVTVSWNSGALT
SGVHTFPAVLQSSGLYSLSSVVTVPSSSLGTKTYTCNVNHKPSNTKVDKRVESKY
GPPCPPCPAPEFLGGPSVFLFPPKPKDTLMISRTPEVTCVVVDVSQEDPEVKFNWY
VDGVEVHNAKTKPREEQFNSTYRVVSVLTVLHQDWLNGKEYKCKVSNKGLPSSI
EKTISKAKGQPREPQVYTLPPSREEMTKNQVSLTCLVKGFYPSDICVEWESNGQP
ENNYKTTPPVLDSDGSFFLYSRLTVDKSRWQQGNVFSCSVMHEALHNHYTQKSL
SLSLG
SEQ ID NO: 10 LC for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 51 HC DNA for Ab2
CAGGTACAGCTGGTGGAGTCTGGGGGAGGCTTGGTACAGCCTGGGGGGTCCC
TGAGACTCTCCTGTGTCGCCTCTGGATTCACCTTCAGTCATTCTAGCATGAACT
GGGTCCGCCAGGCTCCAGGGAAGGGGCTGGAGTGGGTTTCATACATTAGTCG
TGCTACTGGTGCCGTCTACTACGCAGACTCTGTAAAGGGCCGATTCACCATCT
CCAGAGATAATGCCAAAAACTCACTGTATCTGCAAATGAACAGCCTGAGAGA
CGAGGACACGGCTGTGTATTACTGTGCGAGAGAGCCGGTTTTTGACTACTGG
GGCCAGGGAACCCTGGTCACCGTCTCCTCAGCCTCCACCAAGGGCCCATGCG
TCTTCCCGCTAGCGCCCTGCTCCAGGAGCACCTCCGGCAGCACAGCCGCCCTG
GGCTGCCTGGTCAAGGACTACTTCCCCGAACCGGTGACGGTGTCGTGGAACT
CAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCAC
GAAGACCTACACCTGCAACGTAAACCACAAGCCCAGCAACACCAAGGTGGAC
AAGAGAGTTGAGTCCAAATATGGTCCCCCATGCCCACCCTGCCCAGCACCTG
AGTTCCTGGGGGGACCATCAGTCTTCCTGTTCCCCCCAAAACCCAAGGACACT
CTCATGATCTCCCGGACCCCTGAGGTCACGTGCGTGGTGGTGGACGTGAGCC
AGGAAGACCCCGAGGTCAAGTTCAACTGGTACGTGGATGGCGTGGAGGTGCA
TAATGCCAAGACAAAGCCGCGGGAGGAGCAGTTCAACAGCACGTACCGTGTG
GTCAGCGTCCTCACCGTCCTGCACCAGGACTGGCTGAACGGCAAGGAGTACA
AGTGCAAGGTCTCCAACAAAGGCCTCCCGTCCTCCATCGAGAAAACCATCTC
CAAAGCCAAAGGGCAGCCCCGAGAGCCACAGGTGTACACCCTGCCCCCATCC
CGAGAGGAGATGACCAAGAACCAGGTCAGCCTGACCTGCCTGGTCAAAGGCT
TCTACCCCAGCGACATCTGCGTGGAGTGGGAAAGCAATGGGCAGCCGGAGAA
CAACTACAAGACCACGCCTCCCGTGCTGGACTCCGACGGCTCCTTCTTCCTCT
ACAGCAGGCTAACCGTGGACAAGAGCAGGTGGCAGCAGGGGAATGTCTTCTC
ATGCTCCGTGATGCATGAGGCTCTGCACAACCACTACACACAGAAGAGCCTC
TCCCTGTCTCTGGGT
SEQ ID NO: 12 LC DNA for Ab1, Ab2, Ab3, Ab4, Ab5, Ab6, Ab7, and Ab8
GACATCCAGATGACCCAGTCTCCATCTGCCATGTCTGCATCTGTGGGAGACAG
AGTCACCATCACTTGTCGGGCGAGTCAGGACATTAGCAATTATTTAGCCTGGT
TTCAGCAGAAACCAGGGAAAGTCCCTACGCGCCTGATCTATGCTGCATCCAG
TTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTATTACTG
TCTACAGTGGTCCAGTTACCCTCGGACGTTCGGCCAAGGGACCAAGGTGGAA
ATCAAACGGACCGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
Ab3
SEQ ID NO: 1 HCDR1 (North) for Ab1, Ab2, Ab3, Ab4, Ab5, Ab6, Ab7, and Ab8
VASGFTFSHSSMN
SEQ ID NO: 2 HCDR2 (North) for Ab1, Ab2, Ab3, Ab4, Ab5, Ab6, Ab7, Ab8, and
Ab9
YISRATGAVY
SEQ ID NO: 3 HCDR3 (North) for Ab1, Ab2, Ab3, Ab4, Ab5, Ab6, Ab7, Ab8, Ab9,
and Ab10
AREPVFDY
SEQ ID NO: 4 LCDR1 (North) for Ab1, Ab2, Ab3, Ab4, Ab5, Ab6, Ab7, and Ab8
RASQDISNYLA
SEQ ID NO: 5 LCDR2 (North) for Ab1, Ab2, Ab3, Ab4, Ab5, Ab6, Ab7, Ab8, Ab9,
and Ab10
YAASSLQS
SEQ ID NO: 6 LCDR3 (North) for Ab1, Ab2, Ab3, Ab4, Ab5, Ab6, Ab7, Ab8, and
Ab9
LQWSSYPRT
SEQ ID NO: 7 VH for Ab1, Ab2, Ab3, Ab4, Ab5, Ab6, Ab7, and Ab8
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSS
SEQ ID NO: 8 VL for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIK
SEQ ID NO: 37 HC for Ab3
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSSASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDKRVESKYG
PPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWESNGQPE
NNYKTTPPVLDSDGSFFLYSRLTVDKSRWQEGNVFSCSVMHEALHNHYTQKSLS
LSLG
SEQ ID NO: 10 LC for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 38 HC DNA for Ab3
CAGGTACAGCTGGTGGAGTCTGGGGGAGGCTTGGTACAGCCTGGGGGGTCCC
TGAGACTCTCCTGTGTCGCCTCTGGATTCACCTTCAGTCATTCTAGCATGAACT
GGGTCCGCCAGGCTCCAGGGAAGGGGCTGGAGTGGGTTTCATACATTAGTCG
TGCTACTGGTGCCGTCTACTACGCAGACTCTGTAAAGGGCCGATTCACCATCT
CCAGAGATAATGCCAAAAACTCACTGTATCTGCAAATGAACAGCCTGAGAGA
CGAGGACACGGCTGTGTATTACTGTGCGAGAGAGCCGGTTTTTGACTACTGG
GGCCAGGGAACCCTGGTCACCGTCTCCTCAGCCTCCACCAAGGGCCCATCGG
TCTTCCCGCTAGCGCCCTGCTCCAGGAGCACCTCCGAGAGCACAGCCGCCCTG
GGCTGCCTGGTCAAGGACTACTTCCCCGAACCGGTGACGGTGTCGTGGAACT
CAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCAC
GAAGACCTACACCTGCAACGTAGATCACAAGCCCAGCAACACCAAGGTGGAC
AAGAGAGTTGAGTCCAAATATGGTCCCCCATGCCCACCCTGCCCAGCACCTG
AGTTCCTGGGGGGACCATCAGTCTTCCTGTTCCCCCCAAAACCCAAGGACACT
CTCATGATCTCCCGGACCCCTGAGGTCACGTGCGTGGTGGTGGACGTGAGCC
AGGAAGACCCCGAGGTCCAGTTCAACTGGTACGTGGATGGCGTGGAGGTGCA
TAATGCCAAGACAAAGCCGCGGGAGGAGCAGTTCAACAGCACGTACCGTGTG
GTCAGCGTCCTCACCGTCCTGCACCAGGACTGGCTGAACGGCAAGGAGTACA
AGTGCAAGGTCTCCAACAAAGGCCTCCCGTCCTCCATCGAGAAAACCATCTC
CAAAGCCAAAGGGCAGCCCCGAGAGCCACAGGTGTACACCCTGCCCCCATCC
CAGGAGGAGATGACCAAGAACCAGGTCAGCCTGACCTGCCTGGTCAAAGGCT
TCTACCCCAGCGACATCGCCGTGGAGTGGGAAAGCAATGGGCAGCCGGAGAA
CAACTACAAGACCACGCCTCCCGTGCTGGACTCCGACGGCTCCTTCTTCCTCT
ACAGCAGGCTAACCGTGGACAAGAGCAGGTGGCAGGAGGGGAATGTCTTCTC
ATGCTCCGTGATGCATGAGGCTCTGCACAACCACTACACACAGAAGAGCCTC
TCCCTGTCTCTGGGT
SEQ ID NO: 12 LC DNA for Ab1, Ab2, Ab3, Ab4, Ab5, Ab6, Ab7, and Ab8
GACATCCAGATGACCCAGTCTCCATCTGCCATGTCTGCATCTGTGGGAGACAG
AGTCACCATCACTTGTCGGGCGAGTCAGGACATTAGCAATTATTTAGCCTGGT
TTCAGCAGAAACCAGGGAAAGTCCCTACGCGCCTGATCTATGCTGCATCCAG
TTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTATTACTG
TCTACAGTGGTCCAGTTACCCTCGGACGTTCGGCCAAGGGACCAAGGTGGAA
ATCAAACGGACCGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
Ab4
SEQ ID NO: 1 HCDR1 (North) for Ab1, Ab2, Ab3, Ab4, Ab5, Ab6, Ab7, and Ab8
VASGFTFSHSSMN
SEQ ID NO: 2 HCDR2 (North) for Ab1, Ab2, Ab3, Ab4, Ab5, Ab6, Ab7, Ab8, and
Ab9
YISRATGAVY
SEQ ID NO: 3 HCDR3 (North) for Ab1, Ab2, Ab3, Ab4, Ab5, Ab6, Ab7, Ab8, Ab9,
and Ab10
AREPVFDY
SEQ ID NO: 4 LCDR1 (North) for Ab1, Ab2, Ab3, Ab4, Ab5, Ab6, Ab7, and Ab8
RASQDISNYLA
SEQ ID NO: 5 LCDR2 (North) for Ab1, Ab2, Ab3, Ab4, Ab5, Ab6, Ab7, Ab8, Ab9,
and Ab10
YAASSLQS
SEQ ID NO: 6 LCDR3 (North) for Ab1, Ab2, Ab3, Ab4, Ab5, Ab6, Ab7, Ab8, and
Ab9
LQWSSYPRT
SEQ ID NO: 7 VH for Ab1, Ab2, Ab3, Ab4, Ab5, Ab6, Ab7, and Ab8
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSS
SEQ ID NO: 8 VL for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIK
SEQ ID NO: 31 HC for Ab4
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSSASTKGPCVFPLAPCSRSTSESTAALGCLVKDYFPEPVTVSWNSGALTS
GVHTFPAVLQSSGLYSLSSVVTVPSSSLGTKTYTCNVDHKPSNTKVDKRVESKYG
PPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDICVEWESNGQPE
NNYKTTPPVLDSDGSFFLYSRLTVDKSRWQEGNVFSCSVMHEALHNHYTQKSLS
LSLG
SEQ ID NO: 10 LC for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 32 HC DNA for Ab4
CAGGTACAGCTGGTGGAGTCTGGGGGAGGCTTGGTACAGCCTGGGGGGTCCC
TGAGACTCTCCTGTGTCGCCTCTGGATTCACCTTCAGTCATTCTAGCATGAACT
GGGTCCGCCAGGCTCCAGGGAAGGGGCTGGAGTGGGTTTCATACATTAGTCG
TGCTACTGGTGCCGTCTACTACGCAGACTCTGTAAAGGGCCGATTCACCATCT
CCAGAGATAATGCCAAAAACTCACTGTATCTGCAAATGAACAGCCTGAGAGA
CGAGGACACGGCTGTGTATTACTGTGCGAGAGAGCCGGTTTTTGACTACTGG
GGCCAGGGAACCCTGGTCACCGTCTCCTCAGCCTCCACCAAGGGCCCATGCG
TCTTCCCGCTAGCGCCCTGCTCCAGGAGCACCTCCGAGAGCACAGCCGCCCTG
GGCTGCCTGGTCAAGGACTACTTCCCCGAACCGGTGACGGTGTCGTGGAACT
CAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCAC
GAAGACCTACACCTGCAACGTAGATCACAAGCCCAGCAACACCAAGGTGGAC
AAGAGAGTTGAGTCCAAATATGGTCCCCCATGCCCACCCTGCCCAGCACCTG
AGTTCCTGGGGGGACCATCAGTCTTCCTGTTCCCCCCAAAACCCAAGGACACT
CTCATGATCTCCCGGACCCCTGAGGTCACGTGCGTGGTGGTGGACGTGAGCC
AGGAAGACCCCGAGGTCCAGTTCAACTGGTACGTGGATGGCGTGGAGGTGCA
TAATGCCAAGACAAAGCCGCGGGAGGAGCAGTTCAACAGCACGTACCGTGTG
GTCAGCGTCCTCACCGTCCTGCACCAGGACTGGCTGAACGGCAAGGAGTACA
AGTGCAAGGTCTCCAACAAAGGCCTCCCGTCCTCCATCGAGAAAACCATCTC
CAAAGCCAAAGGGCAGCCCCGAGAGCCACAGGTGTACACCCTGCCCCCATCC
CAGGAGGAGATGACCAAGAACCAGGTCAGCCTGACCTGCCTGGTCAAAGGCT
TCTACCCCAGCGACATCTGCGTGGAGTGGGAAAGCAATGGGCAGCCGGAGAA
CAACTACAAGACCACGCCTCCCGTGCTGGACTCCGACGGCTCCTTCTTCCTCT
ACAGCAGGCTAACCGTGGACAAGAGCAGGTGGCAGGAGGGGAATGTCTTCTC
ATGCTCCGTGATGCATGAGGCTCTGCACAACCACTACACACAGAAGAGCCTC
TCCCTGTCTCTGGGT
SEQ ID NO: 12 LC DNA for Ab1, Ab2, Ab3, Ab4, Ab5, Ab6, Ab7, and Ab8
GACATCCAGATGACCCAGTCTCCATCTGCCATGTCTGCATCTGTGGGAGACAG
AGTCACCATCACTTGTCGGGCGAGTCAGGACATTAGCAATTATTTAGCCTGGT
TTCAGCAGAAACCAGGGAAAGTCCCTACGCGCCTGATCTATGCTGCATCCAG
TTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTATTACTG
TCTACAGTGGTCCAGTTACCCTCGGACGTTCGGCCAAGGGACCAAGGTGGAA
ATCAAACGGACCGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
Ab5
SEQ ID NO: 1 HCDR1 (North) for Ab1, Ab2, Ab3, Ab4, Ab5, Ab6, Ab7, and Ab8
VASGFTFSHSSMN
SEQ ID NO: 2 HCDR2 (North) for Ab1, Ab2, Ab3, Ab4, Ab5, Ab6, Ab7, Ab8, and
Ab9
YISRATGAVY
SEQ ID NO: 3 HCDR3 (North) for Ab1, Ab2, Ab3, Ab4, Ab5, Ab6, Ab7, Ab8, Ab9,
and Ab10
AREPVFDY
SEQ ID NO: 4 LCDR1 (North) for Ab1, Ab2, Ab3, Ab4, Ab5, Ab6, Ab7, and Ab8
RASQDISNYLA
SEQ ID NO: 5 LCDR2 (North) for Ab1, Ab2, Ab3, Ab4, Ab5, Ab6, Ab7, Ab8, Ab9,
and Ab10
YAASSLQS
SEQ ID NO: 6 LCDR3 (North) for Ab1, Ab2, Ab3, Ab4, Ab5, Ab6, Ab7, Ab8, and
Ab9
LQWSSYPRT
SEQ ID NO: 7 VH for Ab1, Ab2, Ab3, Ab4, Ab5, Ab6, Ab7, and Ab8
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSS
SEQ ID NO: 8 VL for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIK
SEQ ID NO: 35 HC for Ab5
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSSASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDKRVESKYG
PPCPPCPAPEFLGGPSVFLFPPKPKDTLMISRTPEVTCVVVDVSQEDPEVKFNWYV
DGVEVHNAKTKPREEQFNSTYRVVSVLTVLHQDWLNGKEYKCKVSNKGLPSSIE
KTISKAKGQPREPQVYTLPPSREEMTKNQVSLTCLVKGFYPSDIAVEWESNGQPE
NNYKTTPPVLDSDGSFFLYSRLTVDKSRWQQGNVFSCSVMHEALHNHYTQKSLS
LSLG
SEQ ID NO: 10 LC for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 36 HC DNA for Ab5
CAGGTACAGCTGGTGGAGTCTGGGGGAGGCTTGGTACAGCCTGGGGGGTCCC
TGAGACTCTCCTGTGTCGCCTCTGGATTCACCTTCAGTCATTCTAGCATGAACT
GGGTCCGCCAGGCTCCAGGGAAGGGGCTGGAGTGGGTTTCATACATTAGTCG
TGCTACTGGTGCCGTCTACTACGCAGACTCTGTAAAGGGCCGATTCACCATCT
CCAGAGATAATGCCAAAAACTCACTGTATCTGCAAATGAACAGCCTGAGAGA
CGAGGACACGGCTGTGTATTACTGTGCGAGAGAGCCGGTTTTTGACTACTGG
GGCCAGGGAACCCTGGTCACCGTCTCCTCAGCCTCCACCAAGGGCCCATCGG
TCTTCCCGCTAGCGCCCTGCTCCAGGAGCACCTCCGAGAGCACAGCCGCCCTG
GGCTGCCTGGTCAAGGACTACTTCCCCGAACCGGTGACGGTGTCGTGGAACT
CAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCAC
GAAGACCTACACCTGCAACGTAGATCACAAGCCCAGCAACACCAAGGTGGAC
AAGAGAGTTGAGTCCAAATATGGTCCCCCATGCCCACCCTGCCCAGCACCTG
AGTTCCTGGGGGGACCATCAGTCTTCCTGTTCCCCCCAAAACCCAAGGACACT
CTCATGATCTCCCGGACCCCTGAGGTCACGTGCGTGGTGGTGGACGTGAGCC
AGGAAGACCCCGAGGTCAAGTTCAACTGGTACGTGGATGGCGTGGAGGTGCA
TAATGCCAAGACAAAGCCGCGGGAGGAGCAGTTCAACAGCACGTACCGTGTG
GTCAGCGTCCTCACCGTCCTGCACCAGGACTGGCTGAACGGCAAGGAGTACA
AGTGCAAGGTCTCCAACAAAGGCCTCCCGTCCTCCATCGAGAAAACCATCTC
CAAAGCCAAAGGGCAGCCCCGAGAGCCACAGGTGTACACCCTGCCCCCATCC
CGAGAGGAGATGACCAAGAACCAGGTCAGCCTGACCTGCCTGGTCAAAGGCT
TCTACCCCAGCGACATCGCCGTGGAGTGGGAAAGCAATGGGCAGCCGGAGAA
CAACTACAAGACCACGCCTCCCGTGCTGGACTCCGACGGCTCCTTCTTCCTCT
ACAGCAGGCTAACCGTGGACAAGAGCAGGTGGCAGCAGGGGAATGTCTTCTC
ATGCTCCGTGATGCATGAGGCTCTGCACAACCACTACACACAGAAGAGCCTC
TCCCTGTCTCTGGGT
SEQ ID NO: 12 LC DNA for Ab1, Ab2, Ab3, Ab4, Ab5, Ab6, Ab7, and Ab8
GACATCCAGATGACCCAGTCTCCATCTGCCATGTCTGCATCTGTGGGAGACAG
AGTCACCATCACTTGTCGGGCGAGTCAGGACATTAGCAATTATTTAGCCTGGT
TTCAGCAGAAACCAGGGAAAGTCCCTACGCGCCTGATCTATGCTGCATCCAG
TTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTATTACTG
TCTACAGTGGTCCAGTTACCCTCGGACGTTCGGCCAAGGGACCAAGGTGGAA
ATCAAACGGACCGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
Ab6
SEQ ID NO: 1 HCDR1 (North) for Ab1, Ab2, Ab3, Ab4, Ab5, Ab6, Ab7, and Ab8
VASGFTFSHSSMN
SEQ ID NO: 2 HCDR2 (North) for Ab1, Ab2, Ab3, Ab4, Ab5, Ab6, Ab7, Ab8, and
Ab9
YISRATGAVY
SEQ ID NO: 3 HCDR3 (North) for Ab1, Ab2, Ab3, Ab4, Ab5, Ab6, Ab7, Ab8, Ab9,
and Ab10
AREPVFDY
SEQ ID NO: 4 LCDR1 (North) for Ab1, Ab2, Ab3, Ab4, Ab5, Ab6, Ab7, and Ab8
RASQDISNYLA
SEQ ID NO: 5 LCDR2 (North) for Ab1, Ab2, Ab3, Ab4, Ab5, Ab6, Ab7, Ab8, Ab9,
and Ab10
YAASSLQS
SEQ ID NO: 6 LCDR3 (North) for Ab1, Ab2, Ab3, Ab4, Ab5, Ab6, Ab7, Ab8, and
Ab9
LQWSSYPRT
SEQ ID NO: 7 VH for Ab1, Ab2, Ab3, Ab4, Ab5, Ab6, Ab7, and Ab8
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSS
SEQ ID NO: 8 VL for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIK
SEQ ID NO: 33 HC for Ab6
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKRVEPKSC
DKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCAVSNKAL
PAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYT
QKSLSLSPGK
SEQ ID NO: 10 LC for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 34 HC DNA for Ab6
CAGGTACAGCTGGTGGAGTCTGGGGGAGGCTTGGTACAGCCTGGGGGGTCCC
TGAGACTCTCCTGTGTCGCCTCTGGATTCACCTTCAGTCATTCTAGCATGAACT
GGGTCCGCCAGGCTCCAGGGAAGGGGCTGGAGTGGGTTTCATACATTAGTCG
TGCTACTGGTGCCGTCTACTACGCAGACTCTGTAAAGGGCCGATTCACCATCT
CCAGAGATAATGCCAAAAACTCACTGTATCTGCAAATGAACAGCCTGAGAGA
CGAGGACACGGCTGTGTATTACTGTGCGAGAGAGCCGGTTTTTGACTACTGG
GGCCAGGGAACCCTGGTCACCGTCTCCTCAGCTAGCACCAAGGGCCCATCGG
TCTTCCCCCTGGCACCCTCCTCCAAGAGCACCTCTGGGGGCACAGCGGCCCTG
GGCTGCCTGGTCAAGGACTACTTCCCCGAACCGGTGACGGTGTCGTGGAACT
CAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCAC
CCAGACCTACATCTGCAACGTGAATCACAAGCCCAGCAACACCAAGGTGGAC
AAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCC
AAGGACACCCTCATGATCTCCCGGACCCCTGAGGTCACATGCGTGGTGGTGG
ACGTGAGCCACGAAGACCCTGAGGTCAAGTTCAACTGGTATGTGGACGGCGT
GGAGGTGCATAATGCCAAGACAAAGCCGCGGGAGGAGCAGTACAACAGCAC
GTACCGTGTGGTCAGCGTCCTCACCGTCCTGCACCAAGACTGGCTGAATGGCA
AGGAGTACAAGTGCGCCGTCTCCAACAAAGCCCTCCCAGCCCCCATCGAGAA
AACCATCTCCAAAGCCAAAGGGCAGCCCCGAGAACCACAGGTGTACACCCTG
CCCCCATCCCGGGAGGAGATGACCAAGAACCAAGTCAGCCTGACCTGCCTGG
TCAAAGGCTTCTATCCCAGCGACATCGCCGTGGAGTGGGAGAGCAATGGGCA
GCCGGAGAACAACTACAAGACCACGCCTCCCGTGCTGGACTCCGACGGCTCC
TTCTTCCTCTATTCCAAGCTCACCGTGGACAAGAGCAGGTGGCAGCAGGGGA
ACGTCTTCTCATGCTCCGTGATGCATGAGGCTCTGCACAACCACTACACGCAG
AAGAGCCTCTCCCTGTCTCCGGGCAAA
SEQ ID NO: 12 LC DNA for Ab1, Ab2, Ab3, Ab4, Ab5, Ab6, Ab7, and Ab8
GACATCCAGATGACCCAGTCTCCATCTGCCATGTCTGCATCTGTGGGAGACAG
AGTCACCATCACTTGTCGGGCGAGTCAGGACATTAGCAATTATTTAGCCTGGT
TTCAGCAGAAACCAGGGAAAGTCCCTACGCGCCTGATCTATGCTGCATCCAG
TTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTATTACTG
TCTACAGTGGTCCAGTTACCCTCGGACGTTCGGCCAAGGGACCAAGGTGGAA
ATCAAACGGACCGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
Ab7
SEQ ID NO: 1 HCDR1 (North) for Ab1, Ab2, Ab3, Ab4, Ab5, Ab6, Ab7, and Ab8
VASGFTFSHSSMN
SEQ ID NO: 2 HCDR2 (North) for Ab1, Ab2, Ab3, Ab4, Ab5, Ab6, Ab7, Ab8, and
Ab9
YISRATGAVY
SEQ ID NO: 3 HCDR3 (North) for Ab1, Ab2, Ab3, Ab4, Ab5, Ab6, Ab7, Ab8, Ab9,
and Ab10
AREPVFDY
SEQ ID NO: 4 LCDR1 (North) for Ab1, Ab2, Ab3, Ab4, Ab5, Ab6, Ab7, and Ab8
RASQDISNYLA
SEQ ID NO: 5 LCDR2 (North) for Ab1, Ab2, Ab3, Ab4, Ab5, Ab6, Ab7, Ab8, Ab9,
and Ab10
YAASSLQS
SEQ ID NO: 6 LCDR3 (North) for Ab1, Ab2, Ab3, Ab4, Ab5, Ab6, Ab7, Ab8, and
Ab9
LQWSSYPRT
SEQ ID NO: 7 VH for Ab1, Ab2, Ab3, Ab4, Ab5, Ab6, Ab7, and Ab8
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSS
SEQ ID NO: 8 VL for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIK
SEQ ID NO: 13 HC for Ab7
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSSASTKGPCVFPLAPSSKSTSGGTAALGCLVKDYFPEPVTVSWNSGALT
SGVHTFPAVLQSSGLYSLSSVVTVPSSSLGTQTYICNVNHKPSNTKVDKRVEPKSC
DKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCAVSNKAL
PAPIEKTISKAKGQPREPQVYTLPPSREEMTKNQVSLTCLVKGFYPSDICVEWESN
GQPENNYKTTPPVLDSDGSFFLYSKLTVDKSRWQQGNVFSCSVMHEALHNHYT
QKSLSLSPGK
SEQ ID NO: 10 LC for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 14 HC DNA for Ab7
CAGGTACAGCTGGTGGAGTCTGGGGGAGGCTTGGTACAGCCTGGGGGGTCCC
TGAGACTCTCCTGTGTCGCCTCTGGATTCACCTTCAGTCATTCTAGCATGAACT
GGGTCCGCCAGGCTCCAGGGAAGGGGCTGGAGTGGGTTTCATACATTAGTCG
TGCTACTGGTGCCGTCTACTACGCAGACTCTGTAAAGGGCCGATTCACCATCT
CCAGAGATAATGCCAAAAACTCACTGTATCTGCAAATGAACAGCCTGAGAGA
CGAGGACACGGCTGTGTATTACTGTGCGAGAGAGCCGGTTTTTGACTACTGG
GGCCAGGGAACCCTGGTCACCGTCTCCTCAGCTAGCACCAAGGGCCCATGCG
TCTTCCCCCTGGCACCCTCCTCCAAGAGCACCTCTGGGGGCACAGCGGCCCTG
GGCTGCCTGGTCAAGGACTACTTCCCCGAACCGGTGACGGTGTCGTGGAACT
CAGGCGCACTGACCAGCGGCGTGCACACCTTCCCGGCTGTCCTACAGTCCTCA
GGACTCTACTCCCTCAGCAGCGTGGTGACCGTGCCCTCCAGCAGCTTGGGCAC
CCAGACCTACATCTGCAACGTGAATCACAAGCCCAGCAACACCAAGGTGGAC
AAGAGAGTTGAGCCCAAATCTTGTGACAAAACTCACACATGCCCACCGTGCC
CAGCACCTGAACTCCTGGGGGGACCGTCAGTCTTCCTCTTCCCCCCAAAACCC
AAGGACACCCTCATGATCTCCCGGACCCCTGAGGTCACATGCGTGGTGGTGG
ACGTGAGCCACGAAGACCCTGAGGTCAAGTTCAACTGGTATGTGGACGGCGT
GGAGGTGCATAATGCCAAGACAAAGCCGCGGGAGGAGCAGTACAACAGCAC
GTACCGTGTGGTCAGCGTCCTCACCGTCCTGCACCAAGACTGGCTGAATGGCA
AGGAGTACAAGTGCGCCGTCTCCAACAAAGCCCTCCCAGCCCCCATCGAGAA
AACCATCTCCAAAGCCAAAGGGCAGCCCCGAGAACCACAGGTGTACACCCTG
CCCCCATCCCGGGAGGAGATGACCAAGAACCAAGTCAGCCTGACCTGCCTGG
TCAAAGGCTTCTATCCCAGCGACATCTGCGTGGAGTGGGAGAGCAATGGGCA
GCCGGAGAACAACTACAAGACCACGCCTCCCGTGCTGGACTCCGACGGCTCC
TTCTTCCTCTATTCCAAGCTCACCGTGGACAAGAGCAGGTGGCAGCAGGGGA
ACGTCTTCTCATGCTCCGTGATGCATGAGGCTCTGCACAACCACTACACGCAG
AAGAGCCTCTCCCTGTCTCCGGGCAAA
SEQ ID NO: 12 LC DNA for Ab1, Ab2, Ab3, Ab4, Ab5, Ab6, Ab7, and Ab8
GACATCCAGATGACCCAGTCTCCATCTGCCATGTCTGCATCTGTGGGAGACAG
AGTCACCATCACTTGTCGGGCGAGTCAGGACATTAGCAATTATTTAGCCTGGT
TTCAGCAGAAACCAGGGAAAGTCCCTACGCGCCTGATCTATGCTGCATCCAG
TTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTATTACTG
TCTACAGTGGTCCAGTTACCCTCGGACGTTCGGCCAAGGGACCAAGGTGGAA
ATCAAACGGACCGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
Ab8
SEQ ID NO: 1 HCDR1 (North) for Ab1, Ab2, Ab3, Ab4, Ab5, Ab6, Ab7, and Ab8
VASGFTFSHSSMN
SEQ ID NO: 2 HCDR2 (North) for Ab1, Ab2, Ab3, Ab4, Ab5, Ab6, Ab7, Ab8, and
Ab9
YISRATGAVY
SEQ ID NO: 3 HCDR3 (North) for Ab1, Ab2, Ab3, Ab4, Ab5, Ab6, Ab7, Ab8, Ab9,
and Ab10
AREPVFDY
SEQ ID NO: 4 LCDR1 (North) for Ab1, Ab2, Ab3, Ab4, Ab5, Ab6, Ab7, and Ab8
RASQDISNYLA
SEQ ID NO: 5 LCDR2 (North) for Ab1, Ab2, Ab3, Ab4, Ab5, Ab6, Ab7, Ab8, Ab9,
and Ab10
YAASSLQS
SEQ ID NO: 6 LCDR3 (North) for Ab1, Ab2, Ab3, Ab4, Ab5, Ab6, Ab7, Ab8, and
Ab9
LQWSSYPRT
SEQ ID NO: 7 VH for Ab1, Ab2, Ab3, Ab4, Ab5, Ab6, Ab7, and Ab8
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSS
SEQ ID NO: 8 VL for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIK
SEQ ID NO: 52 HC for Ab8
QVQLVESGGGLVQPGGSLRLSCVASGFTFSHSSMNWVRQAPGKGLEWVSYISRA
TGAVYYADSVKGRFTISRDNAKNSLYLQMNSLRDEDTAVYYCAREPVFDYWGQ
GTLVTVSSASTKGPCVFPLAPSSKSTSGGTAALGCLVKDYFPEPVTVSWNSGALT
SGVHTFPAVLQSSGLYSLSSVVTVPSSSLGTQTYICNVNHKPSNTKVDKRVEPKSC
DKTHTCPPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKAL
AAPIEKTISKAKGQPREPQVYTLPPSREEMTKNQVSLTCLVKGFYPSDICVEWESN
GQPENNYKTTPPVLDSDGSFFLYSKLTVDKSRWQQGNVFSCSVMHEALHNHYT
QKSLSLSPGK
SEQ ID NO: 10 LC for Ab1, Ab2, Ab3, Ab4, Ab5, Ab6, Ab7, and Ab8
DIQMTQSPSAMSASVGDRVTITCRASQDISNYLAWFQQKPGKVPTRLIYAASSLQ
SGVPSRFSGSGSGTEFTLTISSLQPEDFATYYCLQWSSYPRTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 53 HC DNA for Ab8
CAGGTACAGCTGGTGGAGTCTGGGGGAGGCTTGGTACAGCCTGGGGGGTCCC
TGAGACTCTCCTGTGTCGCCTCTGGATTCACCTTCAGTCATTCTAGCATGAACT
GGGTCCGCCAGGCTCCAGGGAAGGGGCTGGAGTGGGTTTCATACATTAGTCG
TGCTACTGGTGCCGTCTACTACGCAGACTCTGTAAAGGGCCGATTCACCATCT
CCAGAGATAATGCCAAAAACTCACTGTATCTGCAAATGAACAGCCTGAGAGA
CGAGGACACGGCTGTGTATTACTGTGCGAGAGAGCCGGTTTTTGACTACTGG
GGCCAGGGAACCCTGGTCACCGTCTCCTCAGCTAGCACCAAGGGCCCATGCG
TCTTCCCCCTGGCACCCTCCTCCAAGAGCACCTCTGGGGGCACAGCGGCCCTG
GGCTGCCTGGTCAAGGACTACTTCCCCGAACCGGTGACGGTGTCGTGGAACT
CAGGCGCACTGACCAGCGGCGTGCACACCTTCCCGGCTGTCCTACAGTCCTCA
GGACTCTACTCCCTCAGCAGCGTGGTGACCGTGCCCTCCAGCAGCTTGGGCAC
CCAGACCTACATCTGCAACGTGAATCACAAGCCCAGCAACACCAAGGTGGAC
AAGAGAGTTGAGCCCAAATCTTGTGACAAAACTCACACATGCCCACCGTGCC
CAGCACCTGAAGCCGCCGGGGGACCGTCAGTCTTCCTCTTCCCCCCAAAACCC
AAGGACACCCTCATGATCTCCCGGACCCCTGAGGTCACATGCGTGGTGGTGG
ACGTGAGCCACGAAGACCCTGAGGTCAAGTTCAACTGGTATGTGGACGGCGT
GGAGGTGCATAATGCCAAGACAAAGCCGCGGGAGGAGCAGTACAACAGCAC
GTACCGTGTGGTCAGCGTCCTCACCGTCCTGCACCAAGACTGGCTGAATGGCA
AGGAGTACAAGTGCAAGGTCTCCAACAAAGCCCTCGCCGCCCCCATCGAGAA
AACCATCTCCAAAGCCAAAGGGCAGCCCCGAGAACCACAGGTGTACACCCTG
CCCCCATCCCGGGAGGAGATGACCAAGAACCAAGTCAGCCTGACCTGCCTGG
TCAAAGGCTTCTATCCCAGCGACATCTGCGTGGAGTGGGAGAGCAATGGGCA
GCCGGAGAACAACTACAAGACCACGCCTCCCGTGCTGGACTCCGACGGCTCC
TTCTTCCTCTATTCCAAGCTCACCGTGGACAAGAGCAGGTGGCAGCAGGGGA
ACGTCTTCTCATGCTCCGTGATGCATGAGGCTCTGCACAACCACTACACGCAG
AAGAGCCTCTCCCTGTCTCCGGGCAAA
SEQ ID NO: 12 LC DNA for Ab1, Ab2, Ab3, Ab4, Ab5, Ab6, Ab7, and Ab8
GACATCCAGATGACCCAGTCTCCATCTGCCATGTCTGCATCTGTGGGAGACAG
AGTCACCATCACTTGTCGGGCGAGTCAGGACATTAGCAATTATTTAGCCTGGT
TTCAGCAGAAACCAGGGAAAGTCCCTACGCGCCTGATCTATGCTGCATCCAG
TTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTATTACTG
TCTACAGTGGTCCAGTTACCCTCGGACGTTCGGCCAAGGGACCAAGGTGGAA
ATCAAACGGACCGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
Ab9
SEQ ID NO: 42 HCDR1 (North) for Ab9
AASGFTFSHSSMN
SEQ ID NO: 2 HCDR2 (North) for Ab1, Ab2, Ab3, Ab4, Ab5, Ab6, Ab7, Ab8, and
Ab9
YISRATGAVY
SEQ ID NO: 3 HCDR3 (North) for Ab1, Ab2, Ab3, Ab4, Ab5, Ab6, Ab7, Ab8, Ab9,
and Ab10
AREPVFDY
SEQ ID NO: 22 LCDR1 (North) for Ab9 and Ab10
RASQGISNYLA
SEQ ID NO: 5 LCDR2 (North) for Ab1, Ab2, Ab3, Ab4, Ab5, Ab6, Ab7, Ab8, Ab9,
and Ab10
YAASSLQS
SEQ ID NO: 6 LCDR3 (North) for Ab1, Ab2, Ab3, Ab4, Ab5, Ab6, Ab7, Ab8, and
Ab9
LQWSSYPRT
SEQ ID NO: 44 VH for Ab9
QVQLVESGGGLVQPGGSLRLSCAASGFTFSHSSMNWVRQAPGKGLEWVSYISRA
TGAVYYADSVKGRFTISRDNAKNSLYLQMNSLRDEDTAVYYCAREPVFDYWGQ
GTLVTVSS
SEQ ID NO: 45 VL for Ab9
DIQMTQSPSAMSASVGDRVTITCRASQGISNYLAWFQQKPGKVPTRLIYAASSLQ
SGVPSRFSGSGSGTEFTLTISSLQPEDFATYYCLQWSSYPRTFGQGTKVEIK
SEQ ID NO: 46 HC for Ab9
QVQLVESGGGLVQPGGSLRLSCAASGFTFSHSSMNWVRQAPGKGLEWVSYISRA
TGAVYYADSVKGRFTISRDNAKNSLYLQMNSLRDEDTAVYYCAREPVFDYWGQ
GTLVTVSSASTKGPCVFPLAPCSRSTSESTAALGCLVKDYFPEPVTVSWNSGALTS
GVHTFPAVLQSSGLYSLSSVVTVPSSSLGTKTYTCNVDHKPSNTKVDKRVESKYG
PPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDICVEWESNGQPE
NNYKTTPPVLDSDGSFFLYSRLTVDKSRWQEGNVFSCSVMHEALHNHYTQKSLS
LSLG
SEQ ID NO: 47 LC for Ab9
DIQMTQSPSAMSASVGDRVTITCRASQGISNYLAWFQQKPGKVPTRLIYAASSLQ
SGVPSRFSGSGSGTEFTLTISSLQPEDFATYYCLQWSSYPRTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 48 HC DNA for 48
CAGGTACAGCTGGTGGAGTCTGGGGGAGGCTTGGTACAGCCTGGGGGGTCCC
TGAGACTCTCCTGTGCAGCCTCTGGATTCACCTTCAGTCATTCTAGCATGAAC
TGGGTCCGCCAGGCTCCAGGGAAGGGGCTGGAGTGGGTTTCATACATTAGTC
GTGCTACTGGTGCCGTCTACTACGCAGACTCTGTAAAGGGCCGATTCACCATC
TCCAGAGATAATGCCAAAAACTCACTGTATCTGCAAATGAACAGCCTGAGAG
ACGAGGACACGGCTGTGTATTACTGTGCGAGAGAGCCGGTTTTTGACTACTG
GGGCCAGGGAACCCTGGTCACCGTCTCCTCAGCCTCCACCAAGGGCCCATGC
GTCTTCCCGCTAGCGCCCTGCTCCAGGAGCACCTCCGAGAGCACAGCCGCCCT
GGGCTGCCTGGTCAAGGACTACTTCCCCGAACCGGTGACGGTGTCGTGGAAC
TCAGGCGCCCTGACCAGCGGCGTGCACACCTTCCCGGCTGTCCTACAGTCCTC
AGGACTCTACTCCCTCAGCAGCGTGGTGACCGTGCCCTCCAGCAGCTTGGGCA
CGAAGACCTACACCTGCAACGTAGATCACAAGCCCAGCAACACCAAGGTGGA
CAAGAGAGTTGAGTCCAAATATGGTCCCCCATGCCCACCCTGCCCAGCACCT
GAGTTCCTGGGGGGACCATCAGTCTTCCTGTTCCCCCCAAAACCCAAGGACAC
TCTCATGATCTCCCGGACCCCTGAGGTCACGTGCGTGGTGGTGGACGTGAGCC
AGGAAGACCCCGAGGTCCAGTTCAACTGGTACGTGGATGGCGTGGAGGTGCA
TAATGCCAAGACAAAGCCGCGGGAGGAGCAGTTCAACAGCACGTACCGTGTG
GTCAGCGTCCTCACCGTCCTGCACCAGGACTGGCTGAACGGCAAGGAGTACA
AGTGCAAGGTCTCCAACAAAGGCCTCCCGTCCTCCATCGAGAAAACCATCTC
CAAAGCCAAAGGGCAGCCCCGAGAGCCACAGGTGTACACCCTGCCCCCATCC
CAGGAGGAGATGACCAAGAACCAGGTCAGCCTGACCTGCCTGGTCAAAGGCT
TCTACCCCAGCGACATCTGCGTGGAGTGGGAAAGCAATGGGCAGCCGGAGAA
CAACTACAAGACCACGCCTCCCGTGCTGGACTCCGACGGCTCCTTCTTCCTCT
ACAGCAGGCTAACCGTGGACAAGAGCAGGTGGCAGGAGGGGAATGTCTTCTC
ATGCTCCGTGATGCATGAGGCTCTGCACAACCACTACACACAGAAGAGCCTC
TCCCTGTCTCTGGGT
SEQ ID NO: 49 LC DNA for Ab9
GACATCCAGATGACCCAGTCTCCATCTGCCATGTCTGCATCTGTGGGAGACAG
AGTCACCATCACTTGTCGGGCGAGTCAGGGCATTAGCAATTATTTAGCCTGGT
TTCAGCAGAAACCAGGGAAAGTCCCTACGCGCCTGATCTATGCTGCATCCAG
TTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTATTACTG
TCTACAGTGGTCCAGTTACCCTCGGACGTTCGGCCAAGGGACCAAGGTGGAA
ATCAAACGGACCGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
Ab10
SEQ ID NO: 19 HCDR1 (North) for Ab10
AASGFTFSISSMN
SEQ ID NO: 20 HCDR2 (North) for Ab10
YISRATGAIY
SEQ ID NO: 3 HCDR3 (North) for Ab1, Ab2, Ab3, Ab4, Ab5, Ab6, Ab7, Ab8, Ab9,
and Ab10
AREPVFDY
SEQ ID NO: 22 LCDR1 (North) for Ab9 and Ab10
RASQGISNYLA
SEQ ID NO: 5 LCDR2 (North) for Ab1, Ab2, Ab3, Ab4, Ab5, Ab6, Ab7, Ab8, Ab9,
and Ab10
YAASSLQS
SEQ ID NO: 24 LCDR3 (North) for Ab10
LQHNSYPRT
SEQ ID NO: 25 VH for Ab10
QVQLVESGGGLVQPGGSLRLSCAASGFTFSISSMNWVRQAPGKGLEWVSYISRA
TGAIYYADSVKGRFTISRNNAKNSLYLQMNSLRDEDTAVYYCAREPVFDYWGQ
GTLVTVSS
SEQ ID NO: 26 VL for Ab10
DIQMTQSPSAMSASVGDRVTITCRASQGISNYLAWFQQKPGKVPTRLIYAASSLQ
SGVPSRFSGSGSGTEFTLTISSLQPEDFATYYCLQHNSYPRTFGQGTKVEIK
SEQ ID NO: 27 HC for Ab10
QVQLVESGGGLVQPGGSLRLSCAASGFTFSISSMNWVRQAPGKGLEWVSYISRA
TGAIYYADSVKGRFTISRNNAKNSLYLQMNSLRDEDTAVYYCAREPVFDYWGQ
GTLVTVSSASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDKRVESKYG
PPCPPCPAPEAAGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHYTQKSL
SLSLG
SEQ ID NO: 28 LC for Ab10
DIQMTQSPSAMSASVGDRVTITCRASQGISNYLAWFQQKPGKVPTRLIYAASSLQ
SGVPSRFSGSGSGTEFTLTISSLQPEDFATYYCLQHNSYPRTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 29 HC DNA for Ab10
CAGGTACAGCTGGTGGAGTCTGGGGGAGGCTTGGTACAGCCTGGGGGGTCCC
TGAGACTCTCCTGTGCAGCCTCTGGATTCACCTTCAGTATCTCTAGCATGAAC
TGGGTCCGCCAGGCTCCAGGGAAGGGGCTGGAGTGGGTTTCATACATTAGTC
GTGCTACTGGTGCCATATACTACGCAGACTCTGTAAAGGGCCGATTCACCATC
TCCAGAAACAATGCCAAAAACTCACTGTATCTGCAAATGAACAGCCTGAGAG
ACGAGGACACGGCTGTGTATTACTGTGCGAGAGAGCCGGTTTTTGACTACTG
GGGCCAGGGAACCCTGGTCACCGTCTCCTCAGCTTCTACCAAGGGCCCATCG
GTCTTCCCGCTAGCGCCCTGCTCCAGGAGCACCTCCGAGAGCACAGCCGCCCT
GGGCTGCCTGGTCAAGGACTACTTCCCCGAACCGGTGACGGTGTCGTGGAAC
TCAGGCGCCCTGACCAGCGGCGTGCACACCTTCCCGGCTGTCCTACAGTCCTC
AGGACTCTACTCCCTCAGCAGCGTGGTGACCGTGCCCTCCAGCAGCTTGGGCA
CGAAGACCTACACCTGCAACGTAGATCACAAGCCCAGCAACACCAAGGTGGA
CAAGAGAGTTGAGTCCAAATATGGTCCCCCATGCCCACCCTGCCCAGCACCT
GAGGCCGCCGGGGGACCATCAGTCTTCCTGTTCCCCCCAAAACCCAAGGACA
CTCTCATGATCTCCCGGACCCCTGAGGTCACGTGCGTGGTGGTGGACGTGAGC
CAGGAAGACCCCGAGGTCCAGTTCAACTGGTACGTGGATGGCGTGGAGGTGC
ATAATGCCAAGACAAAGCCGCGGGAGGAGCAGTTCAACAGCACGTACCGTGT
GGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCT
CCAAAGCCAAAGGGCAGCCCCGAGAGCCACAGGTGTACACCCTGCCCCCATC
CCAGGAGGAGATGACCAAGAACCAGGTCAGCCTGACCTGCCTGGTCAAAGGC
TTCTACCCCAGCGACATCGCCGTGGAGTGGGAAAGCAATGGGCAGCCGGAGA
ACAACTACAAGACCACGCCTCCCGTGCTGGACTCCGACGGCTCCTTCTTCCTC
TACAGCAGGCTAACCGTGGACAAGAGCAGGTGGCAGGAGGGGAATGTCTTCT
CATGCTCCGTGATGCATGAGGCTCTGCACAACCACTACACACAGAAGAGCCT
CTCCCTGTCTCTGGGT
SEQ ID NO: 30 LC DNA for Ab10
GACATCCAGATGACCCAGTCTCCATCTGCCATGTCTGCATCTGTGGGAGACAG
AGTCACCATCACTTGTCGGGCGAGTCAGGGCATTAGCAATTATTTAGCCTGGT
TTCAGCAGAAACCAGGGAAAGTCCCTACGCGCCTGATCTATGCTGCATCCAG
TTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTATTACTG
TCTACAGCATAATAGTTACCCTCGGACGTTCGGCCAAGGGACCAAGGTGGAA
ATCAAACGAACTGTGGCGGCGCCATCTGTCTTCATCTTCCCGCCATCTGATGA
GCAGTTGAAATCCGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
SEQ ID NO: 15 Human IL-4Rα extra-cellular domain
MKVLQEPTCVSDYMSISTCEWKMNGPTNCSTELRLLYQLVFLLSEAHTCIPENNG
GAGCVCHLLMDDVVSADNYTLDLWAGQQLLWKGSFKPSEHVKPRAPGNLTVH
TNVSDTLLLTWSNPYPPDNYLYNHLTYAVNIWSENDPADFRIYNVTYLEPSLRIA
ASTLKSGISYRARVRAWAQCYNTTWSEWSPSTKWHNSYREPFEQH
SEQ ID NO: 16 Cynomolgus monkey IL-4Rα extra-cellular domain
MKVLQEPTCVSDYMSISTCEWKMGGPTNCSAELRLLYQLVFQSSETHTCVPENN
GGVGCVCHLLMDDVVSMDNYTLDLWAGQQLLWKGSFKPSEHVKPRAPGNLTV
HTNVSDTVLLTWSNPYPPDNYLYNDLTYAVNIWSENDPAYSRIHNVTYLKPTLRI
PASTLKSGISYRARVRAWAQHYNTTWSEWSPSTKWYNSYREPFEQR
SEQ ID NO: 17 Human IL-4
MGLTSQLLPPLFFLLACAGNFVHGHKCDITLQEIIKTLNSLTEQKTLCTELTVTDIF
AASKNTTEKETFCRAATVLRQFYSHHEKDTRCLGATAQQFHRHKQLIRFLKRLD
RNLWGLAGLNSCPVKEANQSTLENFLERLKTIMREKYSKCSS
SEQ ID NO: 18 Human IL-13
MHPLLNPLLLALGLMALLLTTVIALTCLGGFASPGPVPPSTALRELIEELVNITQN
QKAPLCNGSMVWSINLTAGMYCAALESLINVSGCSAIEKTQRMLSGFCPHKVSA
GQFSSLHVRDTKIEVAQFVKDLLLHLKKLFREGREN
SEQ ID NO: 39 Human IL-4Rα
MGWLCSGLLFPVSCLVLLQVASSGNMKVLQEPTCVSDYMSISTCEWKMNGPTN
CSTELRLLYQLVFLLSEAHTCIPENNGGAGCVCHLLMDDVVSADNYTLDLWAGQ
QLLWKGSFKPSEHVKPRAPGNLTVHTNVSDTLLLTWSNPYPPDNYLYNHLTYAV
NIWSENDPADFRIYNVTYLEPSLRIAASTLKSGISYRARVRAWAQCYNTTWSEWS
PSTKWHNSYREPFEQHLLLGVSVSCIVILAVCLLCYVSITKIKKEWWDQIPNPARS
RLVAIIIQDAQGSQWEKRSRGQEPAKCPHWKNCLTKLLPCFLEHNMKRDEDPHK
AAKEMPFQGSGKSAWCPVEISKTVLWPESISVVRCVELFEAPVECEEEEEVEEEK
GSFCASPESSRDDFQEGREGIVARLTESLFLDLLGEENGGFCQQDMGESCLLPPSG
STSAHMPWDEFPSAGPKEAPPWGKEQPLHLEPSPPASPTQSPDNLTCTETPLVIAG
NPAYRSFSNSLSQSPCPRELGPDPLLARHLEEVEPEMPCVPQLSEPTTVPQPEPET
WEQILRRNVLQHGAAAAPVSAPTSGYQEFVHAVEQGGTQASAVVGLGPPGEAG
YKAFSSLLASSAVSPEKCGFGASSGEEGYKPFQDLIPGCPGDPAPVPVPLFTFGLD
REPPRSPQSSHLPSSSPEHLGLEPGEKVEDMPKPPLPQEQATDPLVDSLGSGIVYSA
LTCHLCGHLKQCHGQEDGGQTPVMASPCCGCCCGDRSSPPTTPLRAPDPSPGGV
PLEASLCPASLAPSGISEKSKSSSSFHPAPGNAQSSSQTPKIVNFVSVGPTYMRVS
SEQ ID NO: 40 Cynomolgus monkey IL-4Rα
MGWLCSGLLFPVSCLVLLQVASSGCSCVSPGSMKVLQEPTCVSDYMSISTCEWK
MGGPTNCSAELRLLYQLVFQSSETHTCVPENNGGVGCVCHLLMDDVVSMDNYT
LDLWAGQQLLWKGSFKPSEHVKPRAPGNLTVHTNVSDTVLLTWSNPYPPDNYL
YNDLTYAVNIWSENDPAYSRIHNVTYLKPTLRIPASTLKSGISYRARVRAWAQHY
NTTWSEWSPSTKWYNSYREPFEQRLLWGVSAACVFILFFCLSCYFSVTKIKKEW
WDQIPNPARSHLVAIIIQDAQESQWEKRSRGQEAAKCPYWKNCLTKLLPCFLEHN
MKRDEDPHKAVKDLPFRGSGKSAWCPVEISKTVLWPESISVVRCVELFEAPVECK
EEEEVEEEKGSFCTSSESNRDDFQEGREGIVARLTESLFLDLLGGENGGFFQQDM
GESCLLPPLGSTSAHVPWDEFPSAGSKEVPPWGKEQPLHQEPSPPASPTQSPDNPT
CTEMPLVISSNPAYRSFSNSLSQSPCPRELGPDPLLARHLEEVDPEMPCAPQLSEPT
TVAPAEPETWEQILRRNVLQHGAAAAPASAPTSGYREFVHAVQQGGIQASAVAG
LGPPGEAGYKAFSSLLASSAVSPGECGFGASSGEEGYKPFQDLTPGCPGDPAPVP
VPLFTFGLDREPPHSPQSSHLPSNSPEHLALEPGEKVEDMQKPPLPPEQATDPLGD
SLGSGIVYSALTCHLCGHLKQCHGQEDGGQAPVVASPCCGCCCGDRSSPPTTPLR
APDPSLGGVPLEASLCPASLAPSGISEKSKSSLSFHPAPGSAQSSSQTPQIVNFVSV
GPTCMRVS
SEQ ID NO: 41 Human CD23
MEEGQYSEIEELPRRRCCRRGTQIVLLGLVTAALWAGLLTLLLLWHWDTTQSLK
QLEERAARNVSQVSKNLESHHGDQMAQKSQSTQISQELEELRAEQQRLKSQDLE
LSWNLNGLQADLSSFKSQELNERNEASDLLERLREEVTKLRMELQVSSGFVCNT
CPEKWINFQRKCYYFGKGTKQWVHARYACDDMEGQLVSIHSPEEQDFLTKHAS
HTGSWIGLRNLDLKGEFIWVDGSHVDYSNWAPGEPTSRSQGEDCVMMRGSGRW
NDAFCDRKLGAWVCDRLATCTPPASEGSAESMGPDSRPDPDGRLPTPSAPLHS

What is claimed is:

1. A conjugate of the Formula: 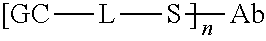
wherein
wherein Ab is an antibody that binds human IL-4Rα, 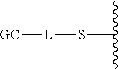
is: 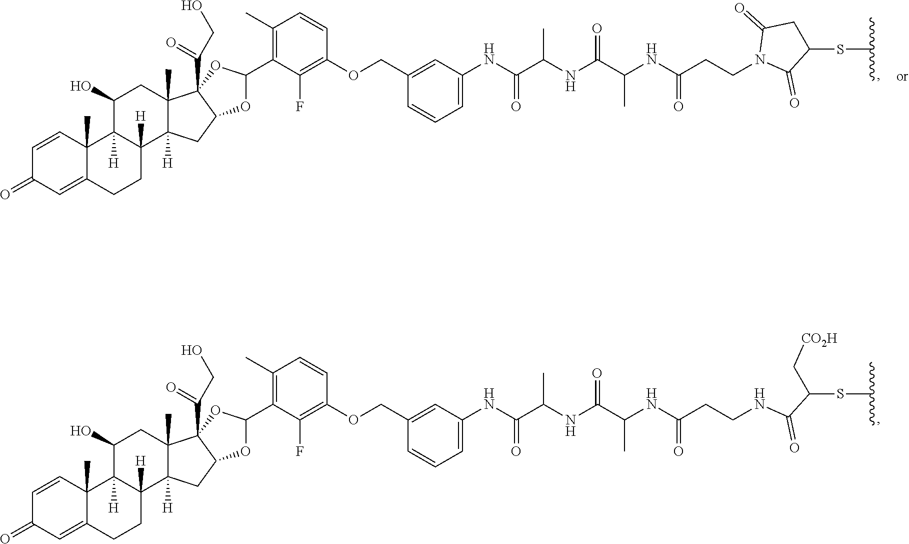
and n is 1-5.

2. The conjugate of claim 1, wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1, 42, or 19;
the HCDR2 comprises SEQ ID NO: 2, or 20;
the HCDR3 comprises SEQ ID NO: 3;
the LCDR1 comprises SEQ ID NO: 4, or 22;
the LCDR2 comprises SEQ ID NO: 5; and
the LCDR3 comprises SEQ ID NO: 6, or 24;

3. The conjugate of claim 1, wherein 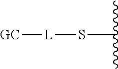
is:

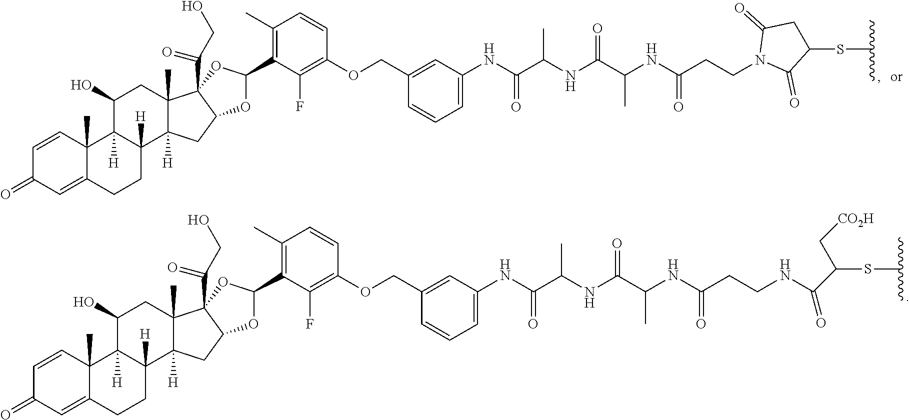

4. The conjugate of claim 1, wherein 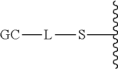
is:

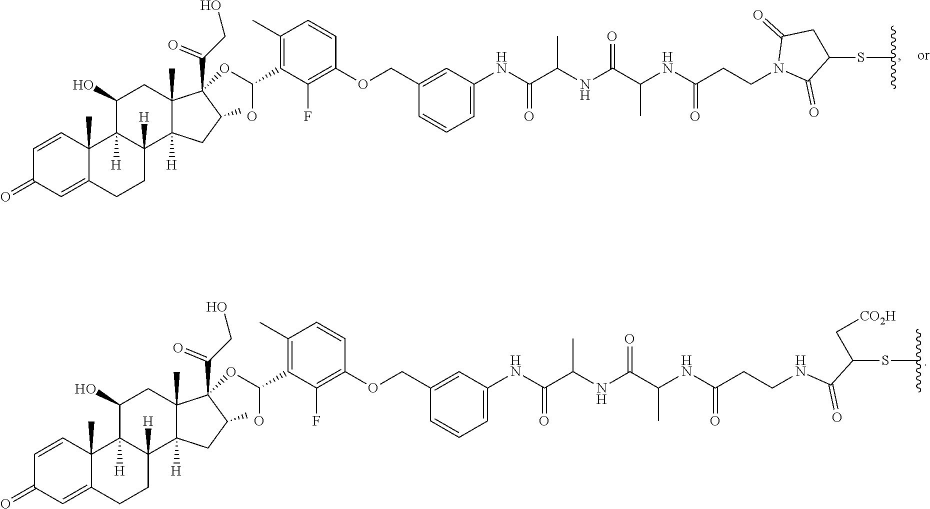

5. The conjugate of claim 1, wherein 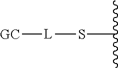
is:

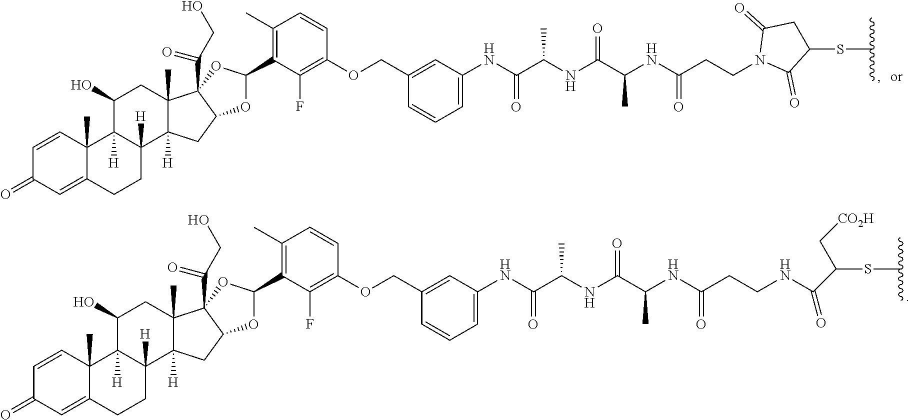

6. The conjugate of claim 1, wherein 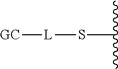
is:

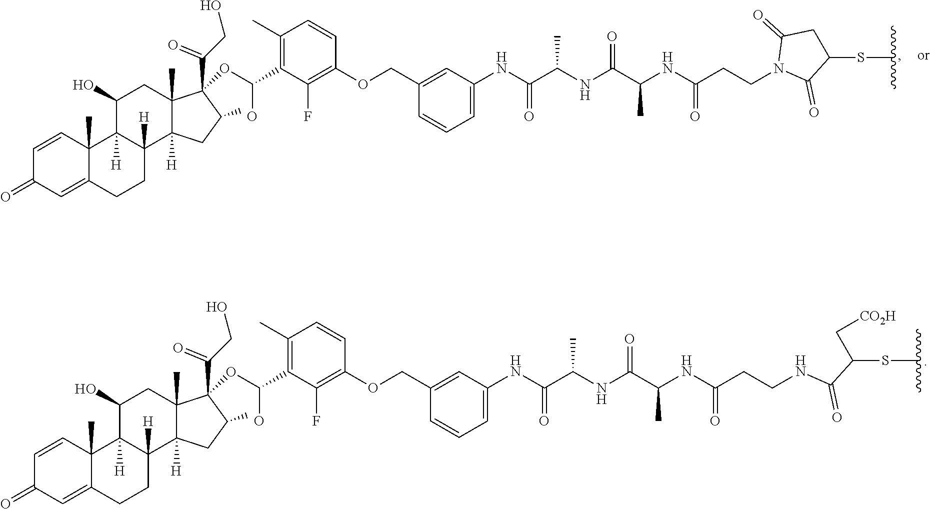

7. The conjugate of claim 1, wherein 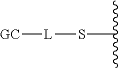
is:

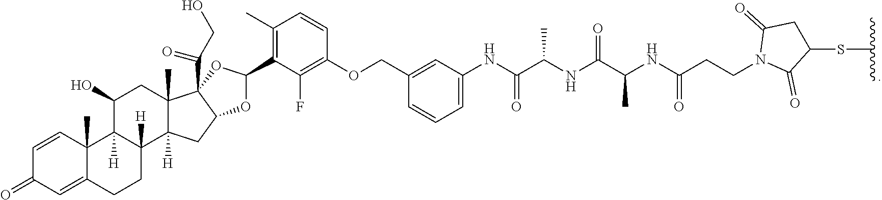

8. The conjugate of claim 1, wherein 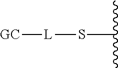
is:

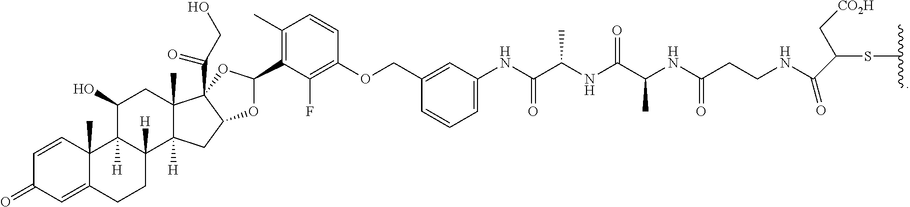

9. The conjugate of claim 1, wherein 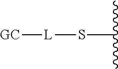
is:

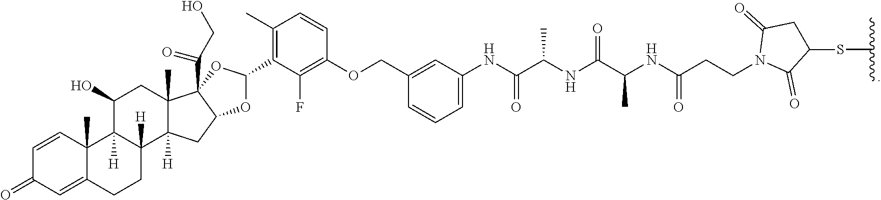

10. The conjugate of claim 1, wherein 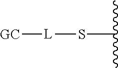
is:

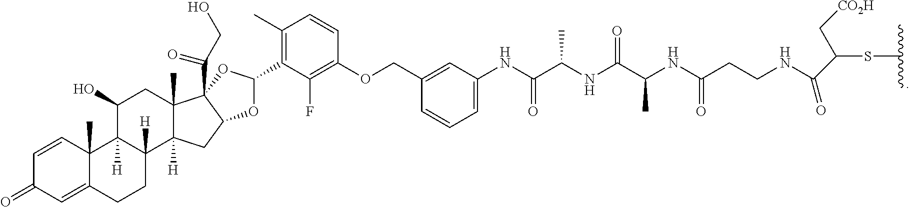

11. The conjugate of claim 1, wherein the Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1,
the HCDR2 comprises SEQ ID NO: 2,
the HCDR3 comprises SEQ ID NO: 3,
the LCDR1 comprises SEQ ID NO: 4,
the LCDR2 comprises SEQ ID NO: 5, and
the LCDR3 comprises SEQ ID NO: 6.

12. The conjugate of claim 11, wherein the VH comprises SEQ ID NO: 7 and the VL comprises SEQ ID NO: 8.

13. The conjugate of claim 11, wherein the Ab comprises:
i. a heavy chain (HC) comprising SEQ ID NO: 9 and a light chain (LC) comprising SEQ ID NO: 10;
ii. a heavy chain (HC) comprising SEQ ID NO: 50 and a light chain (LC) comprising SEQ ID NO: 10;
iii. a heavy chain (HC) comprising SEQ ID NO: 37 and a light chain (LC) comprising SEQ ID NO: 10;
iv. a heavy chain (HC) comprising SEQ ID NO: 31 and a light chain (LC) comprising SEQ ID NO: 10;
v. a heavy chain (HC) comprising SEQ ID NO: 35 and a light chain (LC) comprising SEQ ID NO: 10;
vi. a heavy chain (HC) comprising SEQ ID NO: 33 and a light chain (LC) comprising SEQ ID NO: 10;
vii. a heavy chain (HC) comprising SEQ ID NO: 13 and a light chain (LC) comprising SEQ ID NO: 10; or
viii. a heavy chain (HC) comprising SEQ ID NO: 52 and a light chain (LC) comprising SEQ ID NO: 10.

14. The conjugate of claim 1, wherein the Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 42,
the HCDR2 comprises SEQ ID NO: 2,
the HCDR3 comprises SEQ ID NO: 3,
the LCDR1 comprises SEQ ID NO: 22,
the LCDR2 comprises SEQ ID NO: 5, and
the LCDR3 comprises SEQ ID NO: 6.

15. The conjugate of claim 14, wherein the VH comprises SEQ ID NO: 44 and the VL comprises SEQ ID NO: 45.

16. The conjugate of claim 14, wherein the Ab comprises a heavy chain (HC) comprising SEQ ID NO: 46 and a light chain (LC) comprising SEQ ID NO: 47.

17. The conjugate of claim 1, wherein the Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 19,
the HCDR2 comprises SEQ ID NO: 20,
the HCDR3 comprises SEQ ID NO: 3,
the LCDR1 comprises SEQ ID NO: 22,
the LCDR2 comprises SEQ ID NO: 5, and
the LCDR3 comprises SEQ ID NO: 24.

18. The conjugate of claim 17, wherein the VH comprises SEQ ID NO: 25 and the VL comprises SEQ ID NO: 26.

19. The conjugate of claim 17, wherein the Ab comprises a heavy chain (HC) comprising SEQ ID NO: 27 and a light chain (LC) comprising SEQ ID NO: 28.

20. The conjugate of claim 1, wherein the Ab comprises a heavy chain and a light chain, wherein the heavy chain comprises:
a cysteine at amino acid residue 124 (EU numbering);
a cysteine at amino acid residue 378 (EU numbering); or
a cysteine at amino acid residue 124 (EU numbering) and a cysteine at amino acid residue 378 (EU numbering).

21. The conjugate of claim 1, wherein the Ab comprises a heavy chain (HC) and a light chain (LC), wherein the HC is human IgG4 isotype or human IgG1 isotype.

22. The conjugate of claim 1, wherein n is 2-5.

23. The conjugate of claim 1, wherein n is 3-4.

24. The conjugate of claim 1, wherein n is about 2, 3, or 4.

25. A pharmaceutical composition comprising the conjugate of claim 1 and one or more pharmaceutically acceptable carrier, diluent, or excipient.

26. A method of treating an inflammatory disease in a subject in need thereof, comprising administering to the subject an effective amount of the conjugate of claim 1.

27. A method of treating an inflammatory disease in a subject in need thereof, comprising administering to the subject an effective amount of the pharmaceutical composition of claim 25.

28. The method of claim 26, wherein the inflammatory disease is a type 2 inflammatory disease.

29. The method of claim 28, wherein the type 2 inflammatory disease is atopic dermatitis, eosinophilic esophagitis, nasal polyposis, asthma, chronic rhinosinusitis (CRS), allergic disease, chronic obstructive pulmonary disease (COPD), or chronic spontaneous urticaria (CSU).

30. The method of claim 29, wherein the type 2 inflammatory disease is atopic dermatitis.

31. A method of producing a conjugate, the method comprising contacting the compound of formula 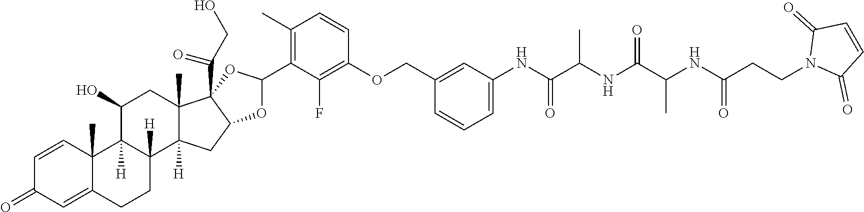
with an anti-human IL-4Rα antibody.

32. The method of claim 31, comprising the steps of:
(a) reducing the anti-human IL-4Rα antibody with a reducing agent to produce a reduced anti-human IL-4R antibody, wherein the anti-human IL-4Rα antibody comprises one or more engineered cysteine residues;
(b) oxidizing the reduced anti-human IL-4Rα antibody with an oxidizing agent to produce an oxidized anti-human IL-4R antibody; and
(c) contacting the oxidized anti-human IL-4R antibody with the compound of formula 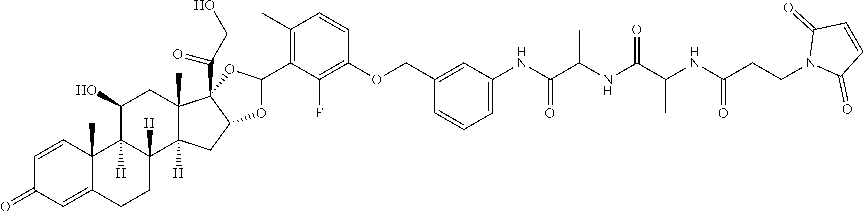
to produce the conjugate.

33. The method of claim 32, wherein the reducing agent is dithiothreitol and the oxidizing agent is dehydroascorbic acid.
